SEQUENCE LISTING (13 sequences)

SEQ ID NO: 1 - protein, 966 aa, Flaveria trinervia
MANRNVEKLASIDAQLRLLVPGKVSEDDKLVEYDALLLDKFLDILQDLHGEDLKEAVQQCYELSAEYEGKHDPKKLEELGSLLTSLDTGDSIVIAKAFSHMLNLANLAEELQIAYRRRIKLKSGDFADEANATTESDIEETFKRLVHKLNKSPEEVFDALKNQTVELVLTAHPTQSVRRSLLQKHGRIRNCLAQLYAKDITPDDKQELDEALHREIQAAFRTDEIRRTPPTPQDEMRAGMSYFHETIWKGVPKFLRRVDTALKNIGINERFPYNAPLIQFSSWMGGDRDGNPRVTPEVTRDVCLLARMMTSNMYFSQIEDLMIEMSMWRCNSELRVRAEELYRTARKDVKHYIEFWKRIPPNQPYRVILGDVRDKLYNTRERSRHLLVDGKSDIPDEAVYTNVEQLLEPLELCYRSLCDCGDHVIADGSLLDFLRQVSTFGLSLVKLDIRQESDRHTEVLDAITQHLGIGSYREWSEEKRQEWLLAELSGKRPLIGPDLPKTEEVKDCLDTFKVLAELPSDCFGAYIISMATSTSDVLAVELLQREYHIKHPLRVVPLFEKLADLEAAPAAMTRLFSMDWYRNRIDGKQEVMIGYSDSGKDAGRFSAAWQLYKTQEQIVKIAKEFGVKLVIFHGRGGTVGRGGGPTHLALLSQPPDTINGSLRVTVQGEVIEQSFGEEHLCFRTLQRFCAATLEHGMNPPISPRPEWRELMDQMAVVATEEYRSVVFKEPRFVEYFRLATPELEFGRMNIGSRPSKRKPSGGIESLRAIPWIFSWTQTRFHLPVWLGFGAAFKHAIQKDSKNLQMLQEMYKTWPFFRVTIDLVEMVFAKGNPGIAALNDKLLVSEDLRPFGESLRANYEETKNYLLKIAGHKDLLEGDPYLKQGIRLRDPYITTLNVCQAYTLKRIRDPNYHVTLRPHISKEYAAEPSKPADELIHLNPTSEYAPGLEDTLILTMKGIAAGMQNTG

SEQ ID NO: 2 - protein, 967 aa, Flaveria pringlei
MANRNLEKLASIDAQLRLLVPGKVSEDDKLIEYDALLLDKFLDILQDLHGEDLKEAVQECYELSAEYEGKHDPKKLEELGSVLTSLDPGDSIVIAKAFSHMLNLANLAEEVQIAYRRRIKLKRGDFADEANATTESDIEETFKKLVLKLNKSPEEVFDALKNQTVDLVLTAHPTQSVRRSLLQKHGRIRNCLAQLYAKDITPDDKQELDEALHREIQAAFRTDEIRRTPPTPQDEMRAGMSYFHETIWKGVPKFLRRVDTALKNIGINERVPYNAPLIQFSSWMGGDRDGKHPRVTPEVTRDVCLLARMMASNMYFSQIEDLMFEMSMWRCNSELRVRAEELYRTARRDVKHYIEFWKQVPPTEPYRVILGDVRDKLYNTRERSRHLLAHGISDIPEEAVYTNVEQFLEPLELCYRSLCDCGDRVIADGSLLDFLRQVSTFGLSLVKLDIRQESDRHTDVLDAITQHLEIGSYREWSEEKRQEWLLAELSGKRPLFGSDLPKTEEVKDVLDTFNVLAELPSDCFGAYIISMATSPSDVLAVELLQRECHVKHPLRVVPLFEKLADLEAAPAAMARLFSIDWYRNRIDGKQEVMIGYSDSGKDAGRFSAAWQLYKAQEEIIKVAKEFGVKLVIFHGRGGTVGRGGGPTHLAILSQPPDTIHGSLRVTVQGEVIEQSFGEEHLCFRTLQRFCAATLEHGMNPPISPRPEWRELMDQMAVVATEEYRSIVFKEPRFVEYFRLATPELEYGRMNIGSRPSKRKPSGGIESLRAIPWIFAWTQTRFHLPVWLGFGAAFKHAIKKDSKNLQMLQEMYKTWPFFRVTIDLVEMVFAKGDPGIAALNDKLLVSEDLWPFGESLRANYEETKDYLLKIAGHRDLLEGDPYLKQRIRLRDSYITTLNVCQAYTLKRIRDPNYHVTLRPHISKEYAAEPSKPADELIHLNPTSEYAPGLEDTLILTMKGIAAGMQNTG

SEQ ID NO: 3 - protein, 33 aa, Hordeum vulgare
PYLKQRLRLRDPYITTLNVCQAYTLKRIRDPSF

SEQ ID NO: 4 - protein, 33 aa, Oryza sativa
LYLKQRLRLRNAYITTLNVCQAYTMKRIRDPDY

SEQ ID NO: 5 - protein, 33 aa, Triticum aestivum
PYLKQRLRLRDAYITTMNVCQAYTLKRIRDPDY

SEQ ID NO: 6 - protein, 33 aa, Arabidopsis thaliana
PYLKQRLRLRDSYITTLNVCQAYTLKRIRDANY

SEQ ID NO: 7 - protein, 33 aa, Flaveria pringlei
PYLKQRIRLRDSYITTLNVCQAYTLKRIRDPNY

SEQ ID NO: 8 - protein, 33 aa, Flaveria trinervia
PYLKQGIRLRDPYITTLNVCQAYTLKRIRDPNY

SEQ ID NO: 9 - protein, 33 aa, Flaveria australasica; identical to SEQ ID NO: 8

SEQ ID NO: 10 - protein, 33 aa, Zea mays
PFLKQGLVLRNPYITTLNVFQAYTLKRIRDPNF

SEQ ID NO: 11 - protein, 33 aa, Sorghum bicolor
PYLKQGLRLRNPYITTLNVFQAYTLKRIRDPSF

SEQ ID NO: 12 - protein, 33 aa, Saccharum spontaneum
PYLKQGLRLRNPYITTLNVLQAYTLKRIRDPSF

SEQ ID NO: 13 - protein, 33 aa, Saccharum officinarum
PYLKQGLRLRNPYITTLNVLQAYTLKRIRDPCF

(19) United States
(12) Patent Application Publication
(10) Pub. No.: US 2015/0216167 A1
(43) Pub. Date: Aug. 6, 2015

(54) SELECTIVE INHIBITION OF C4-PEP CARBOXYLASES

(71) Applicant: Heinrich Heine Universitat Dusseldorf, Dusseldorf (DE)

(72) Inventors: Georg Groth, Julich (DE); Judith Katharina Paulus, Dusseldorf (DE); Daniel Schlieper, Dusseldorf (DE); Peter Westhoff, Neuss (DE)

(21) Appl. No.: 14/367,792

(22) PCT Filed: Dec. 21, 2012
(86) PCT No.: PCT/EP2012/076648

§ 371 Date: Jun. 20, 2014

(30) Foreign Application Priority Data
Dec. 22, 2011 (EP) 11195367.5

(51) Int. Cl. A01N 43/60; G06F 19/18; C07D 241/42; C07D 311/60; C07D 311/28; A01N 43/16; G01N 33/573; C40B 30/02

(52) U.S. Cl. CPC A01N 43/60; G01N 33/573; G06F 19/18; C40B 30/02; C07D 311/60; C07D 311/28; A01N 43/16; C07D 241/42; G01N 2333/988; G01N 2500/20; G01N 2500/04

(57) ABSTRACT

The present invention relates to the use of a compound, a salt or solvate thereof as C4 plant selective herbicide wherein said compound comprises a cyclic alkyl, aryl, heterocycloalkyl or heteroaryl group, and wherein said compound further comprises a) at least a functional group Z being —C(═Y^{z})R¹, wherein Y^{z} is selected from the group consisting of O, NH and S, preferably wherein Y^{z} is O, and wherein R¹ is selected from the group consisting of H, OH and N(R²R³), —O(R²) and —S(R²), wherein R² and R³, are independently of each other, selected from the group consisting of H, alkyl, cycolalkyl, aryl and heteroaryl and a functional group Q which is —C(═Y^{Q})R⁴, wherein Y^{Q} is selected from the group consisting of O, NH and S, preferably wherein Y^{Q} is O, and wherein R⁴ is selected from the group consisting of H, OH and NR^{4#}R^{5#}, OR^{4#}, SR^{4#} wherein R^{4#} and R^{5#}, are independently of each other, selected from the group consisting of H, alkyl, cycloalkyl, aryl and heteroaryl, or b) at least one electron donating group selected from the group consisting of OH, —SH, —O—R^{p}, S—R^{p}—NR^{p}R^{q} and O—C(═Y^{p})R^{r}, wherein R^{p} and R^{q} are, independently of each other, selected from the group consisting of H, alkyl, cycloalkyl and heteroaryl, and wherein R^{r} is —OH, —NH₂, —NH-Alkyl, —O-Alkyl or —NH-Alkyl, and wherein Y^{p} is selected from the group consisting of —S—, —O—, —NH, said compound being capable of binding to the malate binding site comprised by a phosphoenolpyruvate carboxylase from a C4 plant, thereby inhibiting said phosphoenolpyruvate carboxylase, and wherein the cyclic alkyl, aryl, heterocycloalkyl or heteroaryl group inhibits binding to the malate binding site of a phosphoenolpyruvate carboxylase from a C3 plant.

In the past several different targets for selective and non-selective herbicides were identified. However, there still is a great need for new herbicides, especially selective herbicides. In particular specific herbicides against C4 plants would be of outmost importance for the agribusiness and a major turning point in the control of the worst weeds in the world. These weeds reduce the crop yield of the most important C3 cereals such as rice, wheat, barley and oat.

The C4 metabolism offers three potential targets for a selective herbicide. The first target is the enzyme phosphoenol pyruvate (PEP) carboxylase, which has a role in CO₂ fixation.

The second target is the malic enzyme (ME) or the enzyme PEP carboxykinase (PEP CK) which catalyse the release of carbon dioxide in the bundle sheath cells. The third target is the enzyme pyruvate phosphate dikinase (PPDK), which regenerates PEP.

The substances unguinol and ilimaquinone were identified as selective inhibitors of PPDK from C4 plants maize, millet and proso millet. These substances were isolated from marine organisms (Motti et al., 2007). The only universal target, that is common to all variants of C4 metabolism, is the enzyme PEP carboxylase. Due to the high sequence homology between the C4 specific PEP carboxylase and the C3 isoforms, which have further roles in the general C3 metabolism in plants and other organism, it is not obvious to use this enzyme as selective target.

The PEP carboxylase inhibitors described so far (Jenkins et al., 1987; Jenkins, 1989; McFadden et al., 1989; Mancera et al., 1995) are based on PEP analogues (e. g., 3,3-Dichloro-2-(dihydroxyphosphinoylmethyl) propenoate (DCDP)). However, these compounds also inhibit C3 type PEP carboxylases at rates between 12-46% (Jenkins, 1989) and do not inhibit growth of C4 plants (Doyle et al., 2005). Pairoba et al. (1996) showed that PEP carboxylase from C4 plant Amaranthus viridis is inhibited by several flavonoles and flavones. The inhibiting flavonoles are quercetin, quercitrin, rutin, kaempferol, fisetin, morin, and myricetin. The inhibiting flavone was baicalein/baicalin.

But in that work, neither the binding site of the inhibiting flavonoles (or flavones, resp.), nor the molecular mechanism of inhibition were identified. Thus, the mechanism of the inhibiting flavonoles and flavones is unknown. Furthermore, the substances were also described as inhibitors of the malic enzyme from A. viridis, a fact that implies a fairly unspecific inhibition. Furthermore, the authors failed to show that the tested flavonoles and flavones inhibit C4 type PEP carboxylases only, but not C3 type isoforms.

The crystal structures of PEP carboxylase from Escherichia coli (C3 type) and from the C4 plant maize are known (Matsumura et al., 2002). In the publications of these structures, several amino acid side chains were identified as important for the binding of the feedback inhibitors malate and aspartate. However, the authors failed to note the structural difference of the malate binding site that we use in the development of C4 selective herbicides.

Thus, there is a need for effective C4 specific herbicides for all-purpose use, since no selective inhibitors of PEP carboxylase from C4 plants are known.

As set forth above, ilimaquinone and unguinol were identified as inhibitors of PPDK. These inhibitors, however, have several disadvantages, since they inhibit a subgroup of C4 plants. Tropical grasses, which use PEP carboxykinase for CO2 release and PEP regeneration, are not inhibited. Further, the inhibitors need complex biosynthesis pathways and are expensive to produce (100 μg ilimanquinone cost 150 Euro). Also, both compounds have a low solubility in water (high clog values), which hampers the resorption by the plants.

Moreover, the inhibitory concentration of the compounds is high. The half maximal inhibitory concentration (1050) for unguinol is 40 μM for the isolated enzyme. Therefore, an inhibition of the plant would need much higher concentrations up to millimolar range. Due to the high production costs, the use of these compounds is not economical.

Finally, unguinol is used as growth hormone for animals, has anti-microbial and cytotoxic activity and inhibits the enzyme bile salt hydrolase. Therefore, it is unsuitable as herbicide.

For those flavonoles and flavones that were identified as inhibitors of PEP carboxylase from C4 plant A. viridis, the control experiments with C3 plants are missing to show the selective inhibition of the C4 isoform. The title of the publication suggests inhibitory effect of the complete group of flavonoids (Pairoba et al., 1996), but the authors failed to show this claim. Flavonoids are with approx. 7000 different substances the largest class of plant secondary metabolites and have a high structural diversity. From this structurally heterogeneous class of substances, only a few flavonoles and flavones were tested. Flavanones, flavandioles, flavanes, flavanoles, isoflavones and anthocyanes, which also belong to the flavanoides, were not tested.

Furthermore, an inherent disadvantage of naturally occurring flavonoids is their low solubility in water, impeding the application as well the resorption of these substances.

The inherent disadvantage of all tested PEP analogues is the fact that they also inhibit C3 type PEP carboxylases to a certain degree. They do not show any effect on the growth of C4 plants.

Thus, there is a need for effective C4 selective inhibitors. Thus, the present invention relates to use of a compound, a salt or solvate thereof as C4 plant selective herbicide wherein said compound comprises a cyclic alkyl, aryl, heterocycloalkyl or heteroaryl group, and wherein said compound further comprises
(a) at least a functional group Z being —C(═Y^{z})R¹, wherein Y^{z} is selected from the group consisting of O, NE and S, preferably wherein Y^{z} is O, and wherein R¹ is selected from the group consisting of H, OH and N(R²R³), —O(R²) and —S(R²), wherein R² and R³, are, independently of each other, selected from the group consisting of H, Alkyl, cycolalkyl, aryl and heteroaryl and a functional group Q which is —C(═Y^{Q})R⁴, wherein Y^{Q} is selected from the group consisting of O, NH and S, preferably wherein Y^{Q} is O, and wherein R⁴ is selected from the group consisting of H, OH and NR^{4#}R^{5#}, OR^{4#}, SR^{4#} wherein R^{4#} and R^{5#}, are independently of each other, selected from the group consisting of H, alkyl, cycloalkyl, aryl and heteroaryl, or
(b) at least one electron donating group selected from the group consisting of OH, —SH, —O—R^{p}, —S—R^{p}, —NR^{p}R^{q} and —O—C(═Y^{p})R^{r}, wherein R^{p} and R^{q} are, independently of each other, selected from the group consisting of H, alkyl, cycloalkyl and heteroaryl, and wherein R^{r} is —OH, —NH₂, —NH-Alkyl, —O-Alkyl or —NH-Alkyl, and wherein Y^{p} is selected from the group consisting of —S—, —O—, —NH,
   said compound being capable of binding to the malate binding site comprised by a phosphoenolpyruvate carboxylase from a C4 plant, thereby inhibiting said phosphoenolpyruvate carboxylase,
   and wherein the cyclic alkyl, aryl, heterocycloalkyl or heteroaryl group inhibits binding to the malate binding site of a phosphoenolpyruvate carboxylase from a C3 plant.

Within the meaning of the present invention, the term “alkyl” relates to non-branched alkyl residues and branched alkyl residues, as well as residues comprising one or more heteroatoms or functional groups, such as, by way of example, —O—, —S—, —NH—, —NH—C(═O)—, —C(═O)—NH—, and the like. The term also encompasses alkyl groups which are further substituted by one or more suitable substituent.

Within the meaning of the present invention, the term “cyclic alkyl” or “cycloalkyl” relates to optionally suitably substituted 4 to 8-membered single-ring alkyl groups as well as optionally suitably substituted multicyclic alkyl residues.

Within the meaning of the present invention, the term “heterocycloalkyl” relates to optionally suitably substituted 4 to 8-membered single-ring alkyl groups as well as optionally suitably substituted multicyclic alkyl residues comprising one or more, preferably from 1 to 4 such as 1, 2, 3 or 4, heteroatoms, wherein in case the cycloalkyl residue comprises more than 1 heteroatom, the heteroatoms may be the same or different.

The term “alkyl”, “cycloalkyl”, “cyclic alkyl” and “heterocylcoalkyl”, also encompasses groups which are further substituted by one or more suitable substituent.

The term “substituted” as used in this context of the present invention preferably refers to alkyl, cycloalkyl, cyclic alkyl, aryl, heteroaryl and heterocylcoalkyl groups being substituted in any position by one or more substituents, preferably by 1, 2, 3, 4, 5 or 6 substituents, more preferably by 1, 2, or 3 substituents. If two or more substituents are present, each substituent may be the same or may be different from the at least one other substituent. There are in general no limitations as to the substituent.

The substituents may be, for example, selected from the group consisting of aryl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, amino, acylamino, including alkylcarbonylamino, arylcarbonylamino, carbamoyl and ureido, amidino, nitro, imino, sulfhydryl, alkylthio, arylthio, thiocarboxylate, sulfates, alkylsulfinyl, sulfonate, sulfamoyl, sulfonamido, trifluoromethyl, cyano, azido, cycloalkyl such as e.g. cyclopentyl or cyclohexyl, heterocycloalkyl such as e.g. morpholino, piperazinyl or piperidinyl, alkylaryl, arylalkyl and heteroaryl. Preferred substituents of such organic residues are, for example, halogens, such as fluorine, chlorine, bromine or iodine, amino groups, hydroxyl groups, carbonyl groups, thiol groups and carboxyl groups.

Preferred examples for cycloalkyl groups are, e.g., cyclopentyl or cyclohexyl or stereoids, such as, e.g., steroids having a pregnane skeleton, such as 12,20-dioxypregnane-3-yl.

Preferred examples for heterocycloalkyl groups are, e.g., morpholino, piperazinyl or piperidinyl.

Within the meaning of the present invention, the term “aryl” refers to, but is not limited to, optionally suitably substituted 5- and 6-membered single-ring aromatic groups as well as optionally suitably substituted multicyclic groups, for example bicyclic or tricyclic aryl groups. The term “aryl” thus includes, for example, optionally substituted phenyl groups or optionally suitably substituted naphthyl groups. Aryl groups can also be fused or bridged with alicyclic or heterocycloalkyl rings are not aromatic so as to form a polycycle, e.g., benzodioxolyl or tetraline. The term “aryl” further includes aromatic groups which linked via a single bond to further aromatic groups so as to form, e.g., biphenyl groups.

The term “heteroaryl” as used within the meaning of the present invention includes optionally suitably substituted 5- and 6-membered single-ring aromatic groups as well as substituted or unsubstituted multicyclic aryl groups, for example bicyclic or tricyclic aryl groups, comprising one or more, preferably from 1 to 4 such as 1, 2, 3 or 4, heteroatoms, wherein in case the aryl residue comprises more than 1 heteroatom, the heteroatoms may be the same or different. Such heteroaryl groups including from 1 to 4 heteroatoms are, for example, benzodioxolyl, pyrrolyl, furanyl, thiophenyl, thiazolyl, isothiazolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthyridinyl, quinolinyl, isoquinolinyl, indolyl, isoindolyl, benzofuranyl, purinyl, deazapurinyl, pteridinyl, or indolizinyl.

The term “substituted aryl” and the term “substituted heteroaryl” as used in the context of the present invention describes moieties having substituents replacing a hydrogen on one or more atoms, e.g. C or N, of an aryl or heteroaryl moiety. Again, there are in general no limitations as to the substituent. The substituents may be, for example, selected from the group consisting of alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, amino, acylamino, including alkylcarbonylamino, arylcarbonylamino, carbamoyl and ureido, amidino, nitro, imino, sulfhydryl, alkylthio, arylthio, thiocarboxylate, sulfates, alkylsulfinyl, sulfonate, sulfamoyl, sulfonamido, trifluoromethyl, cyano, azido, cycloalkyl such as e.g. cyclopentyl or cyclohexyl, heterocycloalkyl such as e.g. morpholino, piperazinyl or piperidinyl, alkylaryl, arylalkyl and heteroaryl.

Preferred substituents of such organic residues are, for example, halogens, such as fluorine, chlorine, bromine or iodine, amino groups, hydroxyl groups, carbonyl groups, thiol groups, amino groups, and carboxyl groups.

Thus, the cyclic alkyl (cycloalkyl), heterocycloalkyl, aryl or heteroaryl group is, e.g., selected from the group consisting of, optionally suitably substituted, benzodioxolyl, pyrrolyl, furanyl, thiophenyl, thiazolyl, isothiazolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthyridinyl, quinolinyl, isoquinolinyl, indolyl, isoindolyl, benzofuranyl, purinyl, deazapurinyl, pteridinyl, indolizinyl, phenyl, biphenyl and stereoids.

According to a first preferred embodiment, the present invention relates to a use of a compound a salt or solvate thereof as C4 plant selective herbicide wherein said compound comprises a cyclic alkyl, aryl, heterocycloalkyl or heteroaryl group, wherein the compound has a structure according to the formula (I)

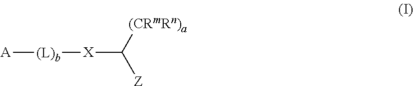

wherein A is the cyclic alkyl, heterocycloalkyl, aryl or heteroaryl group. Preferably, A is an at least bicyclic alkyl, heterocycloalkyl, aryl or heteroaryl group, X is a functional group, L is a linking moiety, integer b is 0 or 1, integer a is in the range of from 1 to 5, preferably 1 to 4, and wherein R^{m} and Rⁿ are, independently of each other, H or alkyl.

The term “at least bicyclic” as used in the context of the present invention refers to groups comprising at least two cycles, wherein these cycles may be fused or may be bridged with alicyclic groups as to form the at least bicyclic group. Each cycle may be selected from aromatic, heteroaromatic and non aromatic cycloalkyl or cycloheteroaryl groups. Term at least bicyclic thus also relates to groups such as biphenyl groups.

According to a preferred embodiment, A is a bicyclic aryl or bicyclic heteroaryl group. The term “bicyclic aryl group” refers to groups in which at least one of both cycles is an aryl group. The term “bicyclic heteroaryl group” refers to groups in which at least one of both cycles is a heteroaryl group.

Preferably, the group A comprises one of the following core structures:

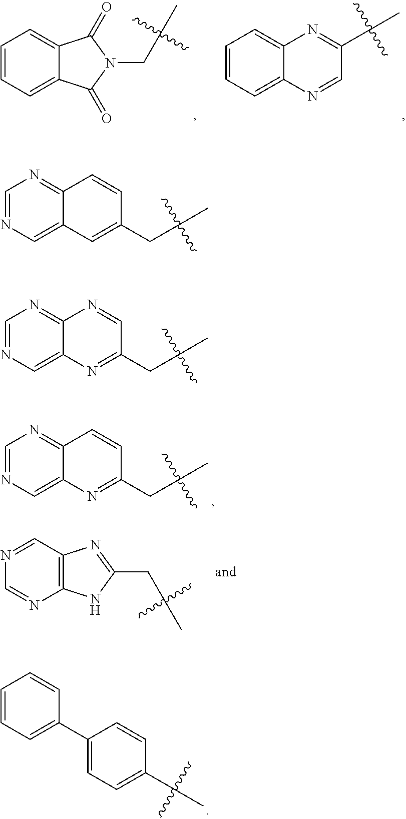

more preferably one of the following core structures

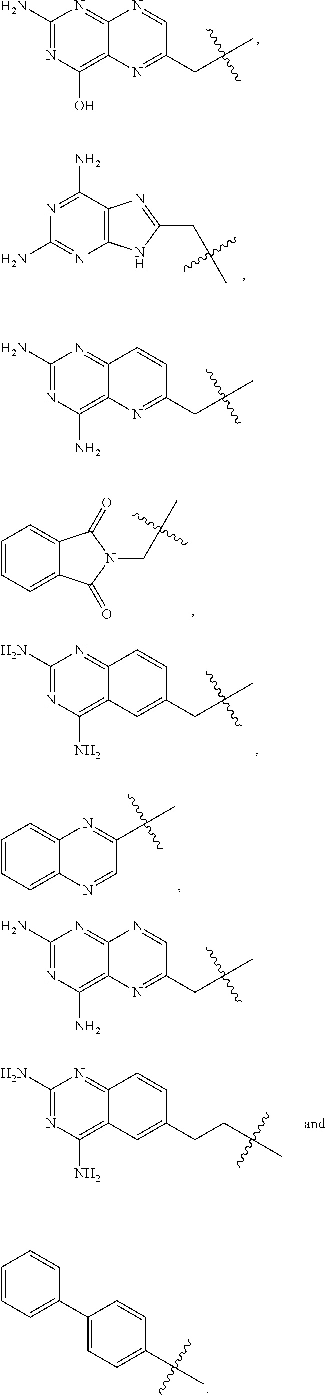

The term “comprising the core structure” as used in the context of the present invention is denoted to mean that the shown structure may be optionally suitably substituted.

Thus, the present invention also relates to the to a use of a compound a salt or solvate thereof as C4 plant selective herbicide according to the formula (1)

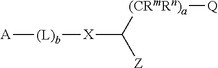

as described above, wherein A comprises one of the following core structures:

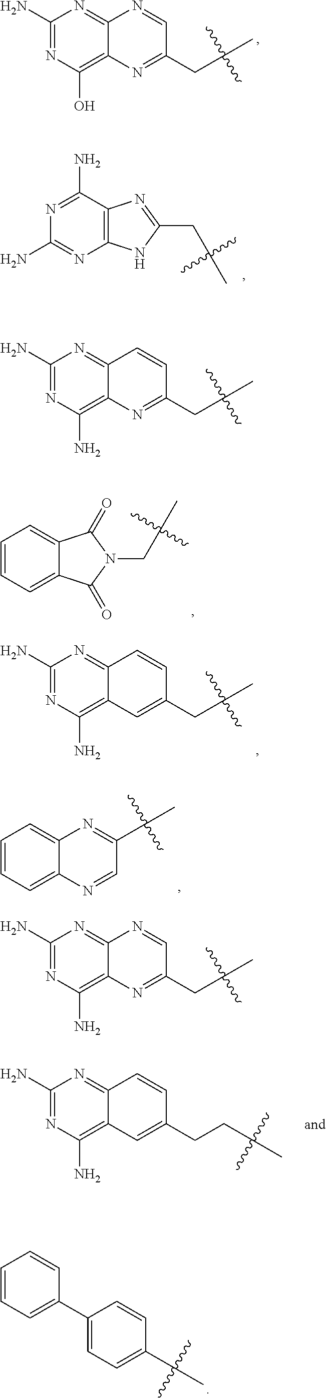

or the core structure:

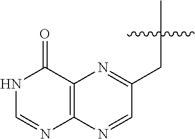 [SMILES: *Cc1cnc2nc[nH]c(=O)c2n1]

In particular, A has a structure selected from the group consisting of

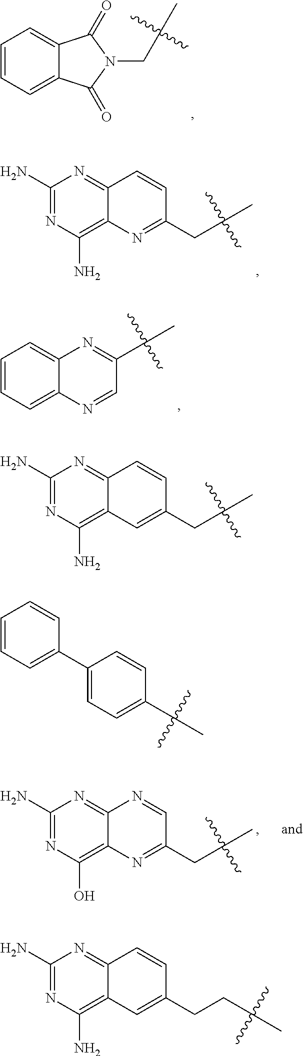

or the structure:

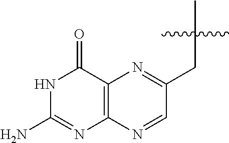 [SMILES: *Cc1cnc2nc(N)[nH]c(=O)c2n1]

more preferably, of the group consisting of

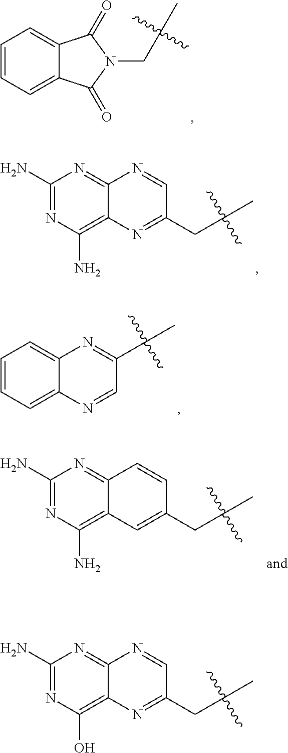 [SMILES: *CN1C(=O)c2ccccc2C1=O ; *Cc1cnc2nc(N)nc(N)c2n1 ; *c1cnc2ccccc2n1 ; *Cc1ccc2nc(N)nc(N)c2c1 ; *Cc1cnc2nc(N)nc(O)c2n1]

### or A is

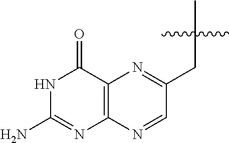 [SMILES: *Cc1cnc2nc(N)[nH]c(=O)c2n1]

Thus, the present invention also relates to the use of a compound a salt or solvate thereof as C4 plant selective herbicide, the compound having a structure according to one of the following formulas:

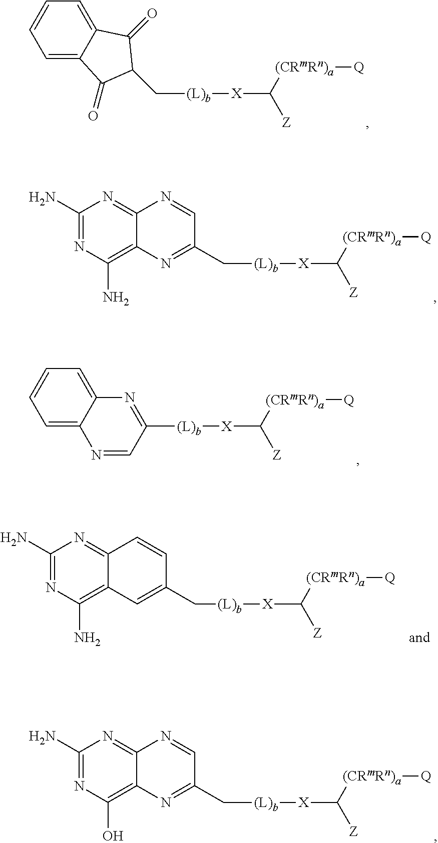

or according to the following formula:

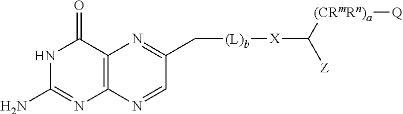

According to an alternative embodiment, A is a stereoid comprising the core structure

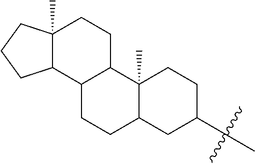 [SMILES: *C1CC[C@@]2(C)C(CCC3C4CCC[C@@]4(C)CCC32)C1]

preferably comprising the core structure

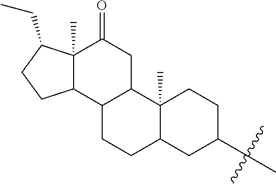 [SMILES: *C1CC[C@@]2(C)C(CCC3C2CC(=O)[C@@]2(C)C3CC[C@@H]2CC)C1]

In case A is a steroid, A is preferably selected from one of the following structures:

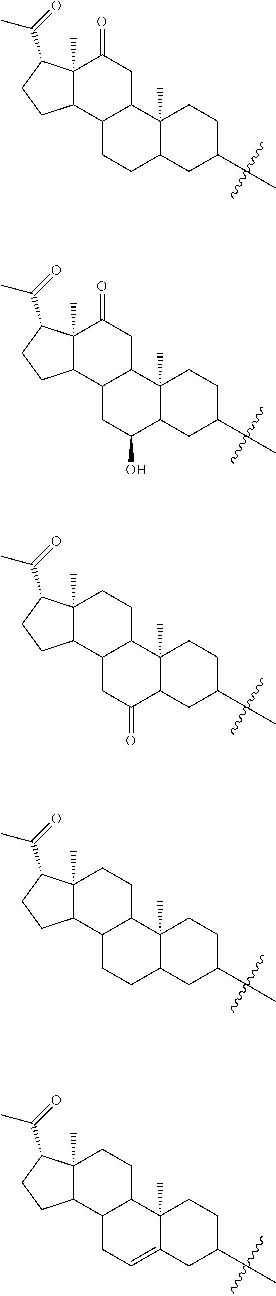 [SMILES: *C1CC[C@@]2(C)C(CCC3C2CC(=O)[C@@]2(C)C3CC[C@@H]2C(C)=O)C1 ; *C1CC[C@]2(C)C3CC(=O)[C@@]4(C)C(CC[C@@H]4C(C)=O)C3C[C@H](O)C2C1 ; *C1CC[C@@]2(C)C(C1)C(=O)CC1C2CC[C@@]2(C)C1CC[C@@H]2C(C)=O ; *C1CC[C@@]2(C)C(CCC3C2CC[C@@]2(C)C3CC[C@@H]2C(C)=O)C1 ; *C1CC[C@@]2(C)C(=CCC3C2CC[C@@]2(C)C3CC[C@@H]2C(C)=O)C1]

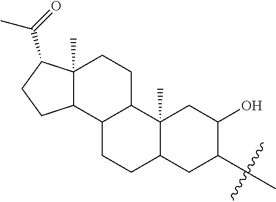 [SMILES: *C1CC2CCC3C(CC[C@@]4(C)C3CC[C@@H]4C(C)=O)[C@@]2(C)CC1O]

more preferably A has the structure

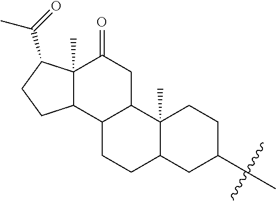 [SMILES: *C1CC[C@@]2(C)C(CCC3C2CC(=O)[C@@]2(C)C3CC[C@@H]2C(C)=O)C1]

Thus, the present invention also relates to the use of a compound a salt or solvate thereof as C4 plant selective herbicide according wherein A has the structure:

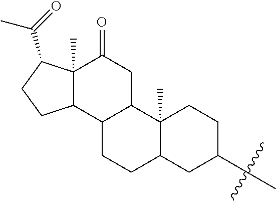 [SMILES: *C1CC[C@@]2(C)C(CCC3C2CC(=O)[C@@]2(C)C3CC[C@@H]2C(C)=O)C1]

and the compound thus has the following structure:

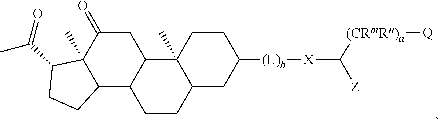

### The Functional Group Q

As described above, the functional group Q is —C(═Y^{Q})R⁴, wherein Y^{Q} is selected from the group consisting of O, NH and S, preferably wherein Y^{Q} is O or S, and wherein R⁴ is selected from the group consisting of H, OH, —O—R^{4#}, —S—R^{4#} and NR^{4#}R^{5#}, wherein R^{4#} and R^{5#}, are independently of each other, selected from the group consisting of H, alkyl, cycloalkyl, aryl, cycloheteroalkyl and heteroaryl.

Thus, the functional group Q is, for example, one of the following groups: —C(═O)H, —C(═O)OH, —C(═O)—O-Alkyl, —C(═O)—O-Aryl, —C(═O)—O— cycloalkyl, —C(═O)—O-heteroaryl, —C(═O)—O-cycloheteroalkyl —C(═O)NH₂, C(═O)NHAlkyl, —C(═O)NHAryl, —C(═O)NHcycloalkyl, —C(═O)NHcycloheteroalkyl, C(═O)N(Hheteroaryl) —C(═S)H, —C(S)OH, —C(═S)—O-Alkyl, —C(═S)—O-Aryl, —C(═O)—O— cycloalkyl, —C(═S)—O-heteroaryl, —C(═S)—O-cycloheteroalkyl.

More preferably Y^{Q} is O. Thus, the functional group Q is, preferably selected from the group consisting of —C(═O)H, —C(═O)OH, —C(═O)—O-Alkyl, —C(═O)—O-Aryl, —C(═O)—O-cycloalkyl, —C(═O)—O-heteroaryl, —C(═O)—O-cycloheteroalkyl —C(═O)NH₂, C(═O)NHAlkyl, —C(═O)NHAryl, —C(═O)NHcycloalkyl, —C(═O)NHcycloheteroalkyl, C(═O)N(Hheteroaryl), More preferably, the functional group Q is selected from the group consisting of —C(═O)H, —C(═O)OH, —C(═O)—O-Alkyl, —C(═O)—O-Aryl, —C(═O)—O-cyclo alkyl, —C(═O)—O-hetero aryl, —C(═O)—O-cycloheteroalkyl.

As to the groups R⁴ and R⁵, these are, independently of each other, H, Alkyl, cycloalkyl, cycloheteroalkyl, aryl or heteroaryl, preferably H or alkyl or cycloalkyl, more preferably H or a linear or branched alkyl group or a cycloalkyl group having from 1 to 10 carbon atoms, preferably an alkyl group or cycloalkyl group selected from the group consisting of methyl, ethyl, propyl, pentyl, butyl, cyclopropyl, cyclobutyl, cyclopropenyl, cyclobutenyl, and hexyl, more preferably H or methyl or ethyl. More preferably one of R^{4#} and R^{5#} is H and one of R^{4#} and R^{5#} is an alkyl group, preferably H or a linear or branched alkyl group having from 1 to 10 carbon atoms, preferably an alkyl group selected from the group consisting of methyl, ethyl, propyl, pentyl, butyl and hexyl, more preferably methyl or ethyl.

In case, Y^{Q} is —C(═O)—O-Alkyl, the alkyl group is preferably a linear or branched alkyl group having from 1 to 10 carbon atoms, preferably an alkyl group selected from the group consisting of methyl, ethyl, propyl, pentyl, butyl and hexyl, more preferably methyl or ethyl.

In case, Y^{Q} is —C(═O)—O-aryl, the aryl group is preferably an optionally substituted phenyl group, preferably phenyl.

Thus, according to a preferred embodiment of the invention, the functional group Q is selected from the group consisting of —C(═O)H, —C(═O)OH, —C(═O)—O-methyl, —C(═O)—O-ethyl, —C(═O)—O-propyl, —C(═O)—O-pentyl, —C(═O)—O-hexyl, —C(═O)—O-phenyl. In particular, the functional group Q is —C(═O)OH, i.e. —COOH, or —C(═O)—O-methyl, most preferably —COOH.

Thus, the present invention also relates to the use, as described above, the compound having the structure (II):

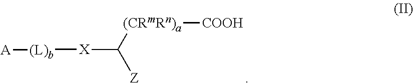

According to an even more preferred embodiment, the present invention also relates to the use of a compound, a salt or solvate thereof as C4 plant selective herbicide according to one of the following formulas:

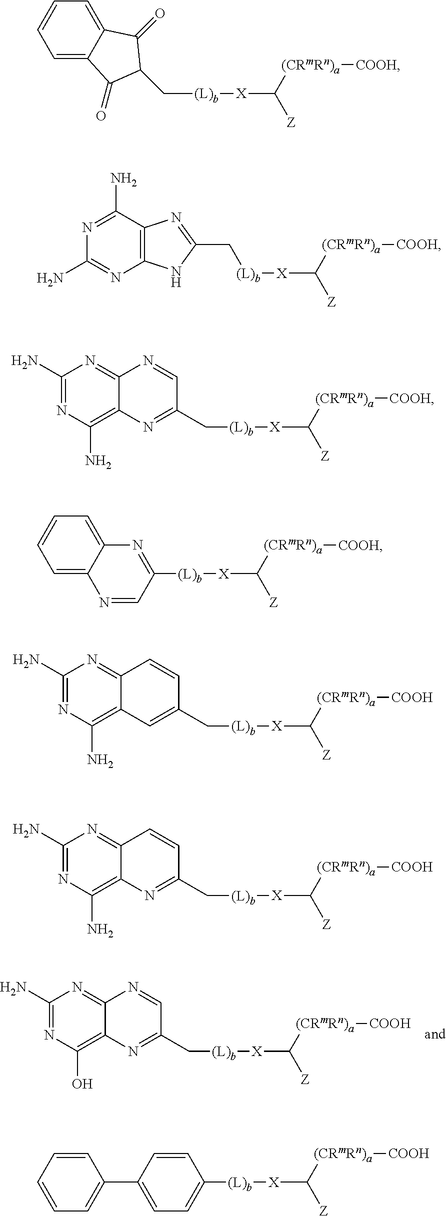

### The Functional Group Z

As described above, the functional group Z is —C(═Y^{z})R¹, wherein Y^{z} is selected from the group consisting of O, NH and S, preferably wherein Y^{z} is O or S, wherein R¹ is selected from the group consisting of H, OH, —O—R², —S—R² and —NR²R³, wherein R² and R³, are independently of each other, selected from the group consisting of H, Aryl, Alkyl, cycloalkyl, heteroaryl and cycloheteroalkyl.

Thus, the functional group Z is, for example, one of the following groups: —C(═O)H, —C(═O)OH, —C(═O)—O-Alkyl, —C(═O)—O-cycloalkyl, —C(═O)—O-heteroaryl, —C(═O)—O-cycloheteroalkyl —C(═O)NH₂, C(═O)NHAlkyl, —C(═O)NHAryl, —C(═O)NHcycloalkyl, —C(═O)NHcycloheteroalkyl, —C(═O)N(Hheteroaryl) —C(═S)H, —C(═S)OH, —C(═S)—O-Alkyl, —C(═S)—O-Aryl, —C(═O)—O— cycloalkyl, —C(═S)—O-heteroaryl, —C(═S)—O-cycloheteroalkyl.

More preferably Y^{z} is O. Thus, the functional group Z is, preferably selected from the group consisting of —C(═O)H, —C(═O)OH, —C(═O)—O-Alkyl, —C(═O)—O-Aryl, —C(═O)—O— cycloalkyl, —C(═O)—O-heteroaryl, —C(═O)—O-cycloheteroalkyl —C(═O)NH₂, C(═O)NHAlkyl, —C(═O)NHAryl, —C(═O)NHcycloalkyl, —C(═O)NHcycloheteroalkyl and C(═O)NHheteroaryl.

As to the groups R² and R³, these are, independently of each other, selected from the group consisting of H, Aryl, Alkyl, cycloalkyl, heteroaryl and cycloheteroalkyl, preferably H or alkyl or cycloalkyl, preferably H or a linear or branched, alkyl group or cycloalkyl group, having from 1 to 10 carbon atoms, preferably an alkyl group or cycloalkyl group selected from the group consisting of methyl, ethyl, propyl, pentyl, butyl, cyclopropyl, cyclobutyl, cyclopropenyl, cyclobutenyl and hexyl. More preferably the groups R² and R³ are, independently of each other, H or methyl or ethyl. More preferably one of R² and R³ is H and one of R² and R³ is an alkyl group or cycloalkyl group, preferably H or a linear or branched alkyl group or cycloalkyl group, having from 1 to 10 carbon atoms, preferably H or an alkyl group selected from the group consisting of methyl, ethyl, propyl, pentyl, butyl and hexyl, more preferably H or methyl or ethyl.

In case, Y^{Z} is —C(═O)—O-Alkyl, the alkyl group is preferably a linear or branched alkyl group having from 1 to 10 carbon atoms, preferably an alkyl group selected from the group consisting of methyl, ethyl, propyl, pentyl, butyl and hexyl, more preferably methyl or ethyl.

In case, Y^{Z} is —C(═O)—O-Aryl, the aryl group is preferably an optionally substituted phenyl group, preferably phenyl.

Thus, according to a preferred embodiment of the invention, the functional group Z is selected from the group consisting of —C(═O)H, —C(═O)OH, —C(═O)—O-methyl, —C(═O)—O-ethyl, —C(═O)—O-propyl, —C(═O)—O-pentyl, —C(═O)—O-hexyl, and —C(═O)—O-phenyl. In particular, the functional group Z is —C(═O)OH, i.e. —COOH, or —C(═O)—O-methyl, most preferably —COOH.

Thus, the present invention also relates to the use, as described above, the compound having the structure (III):

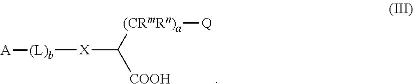

According to an even more preferred embodiment, the present invention also relates to the use of a compound, a salt or solvate thereof as C4 plant selective herbicide according to one of the following formulas:

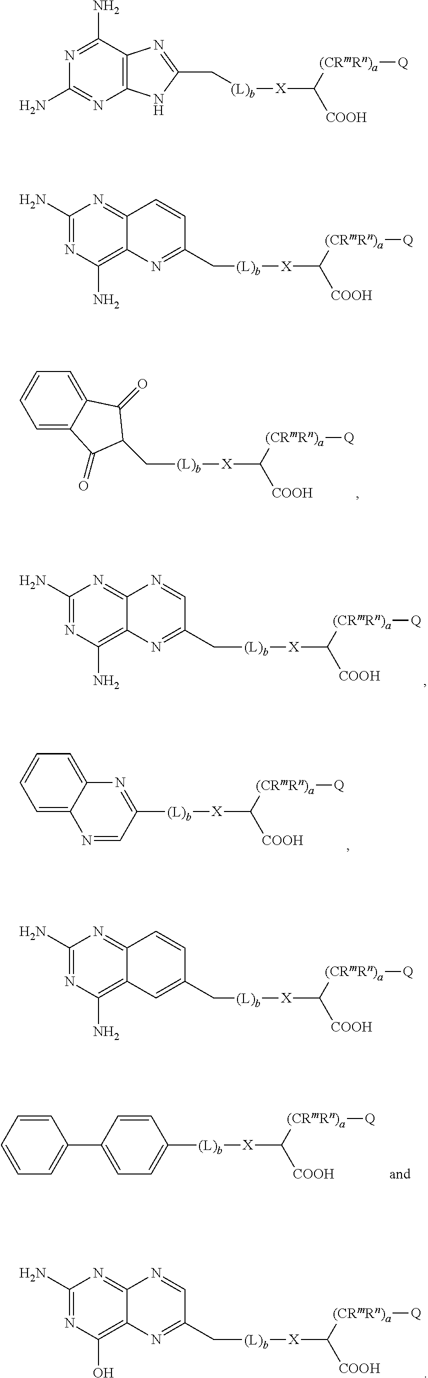

In particular, Z and Q are both —COOH. Thus, the present invention also relates to the use, as described above, the compound having the structure (IV):

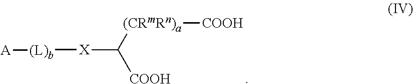

In particular, the present invention also relates to the use of a compound a salt or solvate thereof as C4 plant selective herbicide, the compound having a structure according to one of the following formulas:

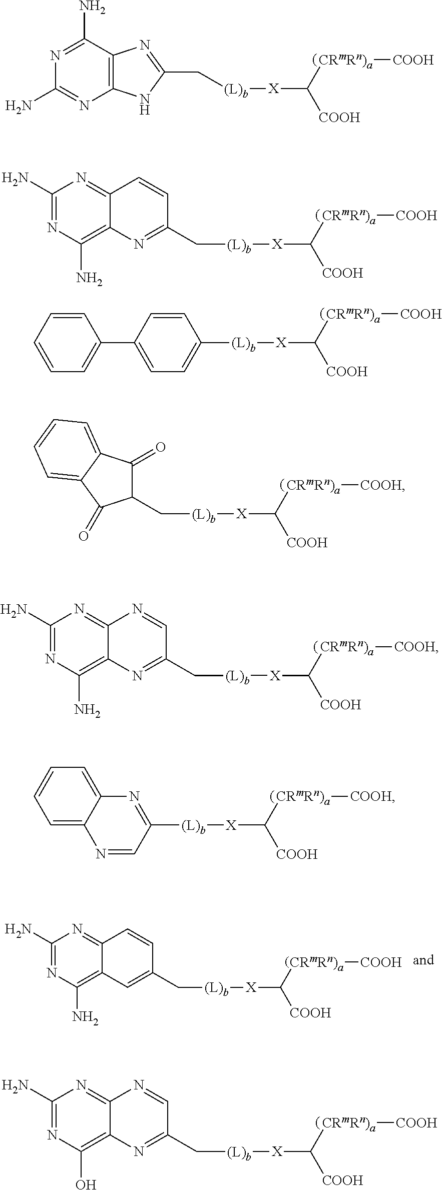

or according to the following formula:

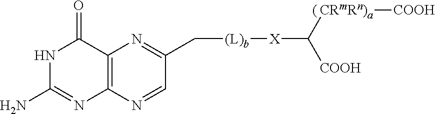

### The Functional Group X

As described above, X is a functional group, linking the moiety A-(L)_{b} and the carbon atom linked to Z and the structural unit (CR^{m}Rⁿ)ₐ-Q.

Preferably X is —Y^{X}— or —C(═Y^{X})—, wherein Y^{X} is selected from the group consisting of S—, —O—, —NH—, —N(CH₃)— and —NH—NH—, and Y^{XX} (is selected from the group consisting of S—, —O—, —NH. Thus, X is preferably selected from the group consisting of —S—, —NH—, -, —N(CH₃)— and —NH—NH—, —C(═O)—, —C(═S) and —C(═NH)—,

In particular X is NH— or —C(═Y)— with Y being S or O,

Thus, the present invention also relates to the use, as described above, wherein X is NH— or —C(═Y)— with Y being S or O, the compound thus having a structure according to one of the following formulas:

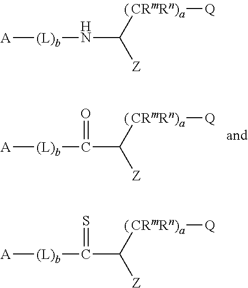

wherein X is most —NH—.

### Integer a

As mentioned above, integer a is in the range of from 1 to 5, such as 1, 2, 3, 4 or 5, preferably in the range of from 1 to 4 In case integer a is greater than 1, each repeating unit [CR^{m}Rⁿ] may be the same or may be different from each other.

Preferably a is in the range of from 1 to 3.

According to a preferred embodiment of the present invention, R^{m} and Rⁿ are, independently of each other, selected from H or branched or linear alkyl groups, comprising 1 to 10, preferably 1 to 8, more preferably 1 to 5, most preferably 1 to 3 carbon atoms. More preferably R^{m} and Rⁿ are, independently of each other, selected from the group consisting of H, methyl, ethyl, propyl, butyl, sec-butyl and tert-butyl, more preferably R^{m} and Rⁿ are, independently of each other, H or methyl.

By way of example, the following preferred structures for the structural unit [CR^{m}Rⁿ]ₐ are mentioned: CH₂—CH₂—CH₂—, CH₂—CH₂—, CH₂—, —CH(CH₃)—, —C(CH₃)₂—, —CH(CH₂CH₃)—, —CH(CH(CH₃)₂)—, —CH(CH₃)—CH₂—, —CH₂—CH(CH₃)—, —CH(CH₃)—CH₂—CH₂—, —CH₂—CH(CH₃)—CH₂—, —CH₂—CH₂—CH(CH₃)—, —CH(CH₃)—CH(CH₃)—, CH(CH₃)—CH(CH₃)—CH₂—, —CH₂—CH(CH₃)—CH(CH₃)—, —CH(CH₃)—CH₂—CH(CH₃)—.

More preferably integer a is 1 or 2, most preferably 2.

According to a particularly preferred embodiment of the present invention, R^{m} and Rⁿ are both H. The structural unit [CR^{m}Rⁿ]ₐ is thus preferably —CH₂—CH₂—CH₂—, CH₂—CH₂— or —CH₂—, more preferably a is 1 or 2, the structural unit [CR^{m}Rⁿ]ₐ thus preferably having the structure CH₂—CH₂— or CH₂—, most preferably a is 2 and the structural unit [CR^{m}Rⁿ]ₐ is —CH₂—CH₂—.

Thus, the present invention also relates to the to a use of a compound a salt or solvate thereof as C4 plant selective herbicide according to the following formula:

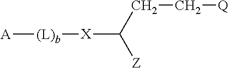

more preferably according to one of the following formulas,

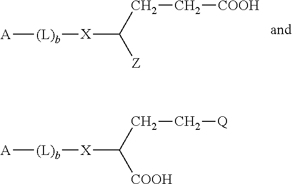

more preferably according to the following formula

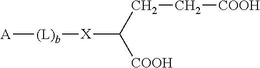

### The Linking Moiety L

As described above, L is a linking moiety. This linking moiety, if present, i.e. if integer b is 1, links A with the functional group X.

Preferably, the linking moiety L has a structure according to the following formula

-[F²]_{c}[L¹]_{d}[F¹]ₑ-

wherein L¹ is a linking moiety linking the structural units —[F²]_{c} and —[F¹]ₑ, wherein L¹ is preferably selected from the group consisting of alkyl, alkenyl, alkylaryl, arylalkyl, aryl, heteroaryl, alkylheteroaryl and heteroarylalkyl, wherein integer d is 0 or 1, and wherein F1 and F2 are functional groups which may be the same or may differ from each other, integer e is 0 or 1 and integer c is 0 or 1.

The term “alkenyl” as used in the context of the present invention refers to unsaturated alkyl groups having at least one double bond. The term also encompasses alkenyl groups which are substituted by one or more suitable substituents.

The term “alkynyl” refers to unsaturated alkyl groups having at least one triple bond. The term also encompasses alkynyl groups which are substituted by one or more suitable substituents.

The term “alkylaryl” as used in the context of the linking moiety described in the present invention is denoted to mean a linking moiety having the structure alkyl-aryl-, thus being linked on one side via the alkyl group and on the other side via the aryl group, wherein this term is meant to also encompass linking moieties such as alkyl-aryl-alkyl-linking moieties.

The term “alkylaryl group”, when used in the context of any substituent described hereinunder and above, is denoted to mean a residue being linked via the alkyl portion, said alkyl portion being further substituted with an aryl moiety.

The term “arylalkyl” as used in the context of any linking moiety described in the present invention is denoted to mean a linking moiety having the structure aryl-alkyl-, thus being linked on one side via the aryl group and on the other side via the alkyl group, wherein this term is meant to also encompass linking moieties such as aryl-alkyl-aryl-linking moieties.

The term “arylalkyl group”, when used in the context of any substituent described hereinunder and above, is denoted to mean a residue being linked via the aryl portion, said aryl portion being further substituted with an alkyl moiety.

The term “alkylheteroaryl” as used in the context of any linking moiety described in the present invention is denoted to mean a linking moiety having the structure alkyl-heteroaryl-, thus being linked on one side via the alkyl group and on the other side via the heteroaryl group, wherein this term is meant to also encompass linking moieties such as alkyl-heteroaryl-alkyl-linking moieties.

The term “alkylheteroaryl group”, when used in the context of any substituent described hereinunder and above, is denoted to mean a residue being linked via the alkyl portion, said alkyl portion being further substituted with a heteroaryl moiety.

The term “heteroarylalkyl” as used in the context of any linking moiety described in the present invention is denoted to mean a linking moiety having the structure heteroaryl-alkyl-, thus being linked on one side via the heteroaryl group and on the other side via the alkyl group, wherein this term is meant to also encompass linking moieties such as heteroaryl-alkyl-heteroaryl-linking moieties.

The term “heteroarylalkyl group”, when used in the context of any substituent described hereinunder and above, is denoted to mean a residue being linked via the heteroaryl portion, said heteroaryl portion being further substituted with an alkyl moiety.

Preferably L¹ is an alkylaryl, arylalkyl, heteroaryl or aryl group, preferably a group having one of the following structures:

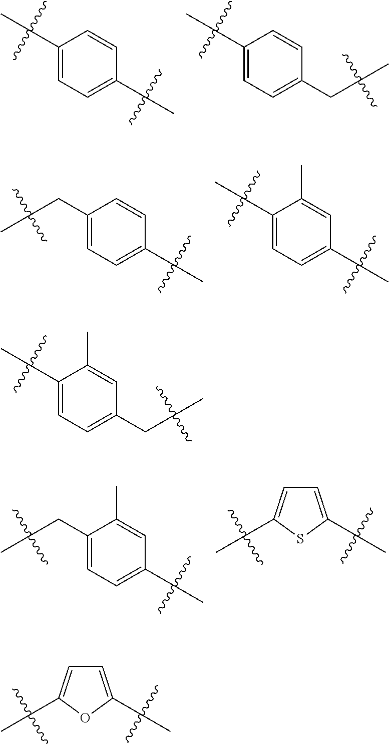

more preferably a group having one of the following structures:

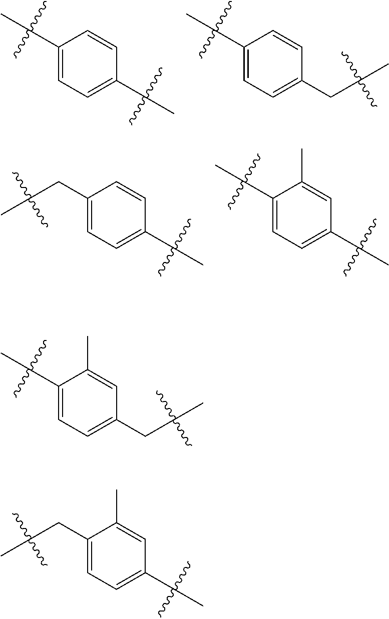

and even more preferably a group having one of the following structures:

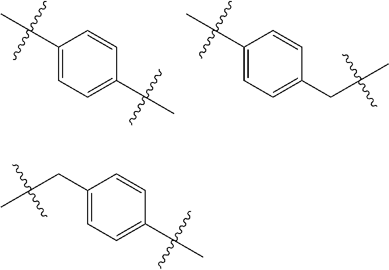 [SMILES: *c1ccc(*)cc1 ; *Cc1ccc(*)cc1 ; *Cc1ccc(*)cc1]

### The Functional Group F¹:

F¹ is a functional group linking X and the structural unit A-[F²]_{c}-[L¹]_{d}. There are, in general, no particular restrictions as regards the chemical nature of the functional group F¹ provided that a stable bond is formed linking X and the structural unit A-[F²]_{c}-[L¹]_{d}. Preferably F¹ is a functional group is a functional group selected from the group consisting of —Y^{F1}—, —C(═Y^{F})—, and —C(═Y^{F})—NR^{Y1}—, wherein Y^{F1} is selected from the group consisting of —S—, —O—, —NH—, —N(CH₃)— and —NH—NH—, and wherein Y^{F} is selected from the group consisting of S, O, NH and R^{Y1} is H or alkyl.

Thus, F¹ is preferably selected from the group consisting of —S—, —O—, —NH—, —N(CH₃)— and NH—NH—, —C(═S), —C(═O)—, —C(═NH)—, —C(═O)NH—, —C(═O)N(alkyl)-, C(═S)N(alkyl), —C(═NH)NH—, —C(NH)N(alkyl).

In case X is —Y^{X}—, F¹ is —C(═Y^{F})— or —C(═Y^{F})—NR^{Y1}.

### The Functional Group F²:

F² is a functional group linking A and the structural unit -[L¹]_{d}-[F¹]ₑ-X. There are, in general, no particular restrictions as regards the chemical nature of the functional group F¹ provided that a stable bond is formed linking A and the structural unit -[L¹]_{d}-[F¹]ₑ-X. Preferably F² is selected from the group consisting of —Y^{F2}—, —C(═Y^{FF})—, —C(═Y^{FF})—NR^{Y2}— and —NR^{Y2}—C(═Y^{FF})—, wherein Y^{F2} is selected from the group consisting of —S—, —O—, —NH—, —N(CH₃)—, —NH—NH—, Y^{FF} is selected from the group consisting of —S—, —O—, —NH and R^{Y2} is H or alkyl.

Thus, F² is preferably selected from the group consisting of —S—, —O—, —NH—, —N(CH₃)—, NH—NH—, C(═S), —C(═O)—, —C(═O)NH—, —C(═O)N(alkyl)—, —C(═S)NH—, —C(═S)N(alkyl), —C(═NH)NH—, —C(═NH)N(alkyl)-. —NHC(═O)—, —N(alkyl)-C(═O)—, NHC(═S), —N(alkyl)C(═S), NHC(═NH) and N(alkyl)C(═NH).

### Preferred Linking Moieties According to the Invention

According to a preferred embodiment of the invention, L has the structure

—Y^{F2}-L¹-C(═Y^{F})—

with Y^{F2}, L¹ and Y^{F2} being as described above. Preferably, in this preferred case, L¹ is aryl or alkylaryl.

In particular L is selected from the following structures:

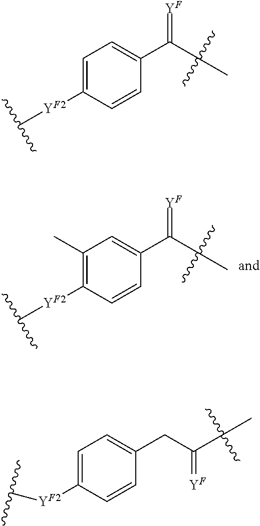

By way of example, the following preferred embodiments for L are described:

TABLE 1
Preferred linking moieties L

| —[F²]_{c}— | —[F¹]ₑ— | —[L¹]_{d}— |
|---|---|---|
| —NH— —N(Me)— —O— —S— —C(═O)—NH— —NH—C(═O)— —NH— —N(Me)— —O— —S— —C(═O)—NH— —NH—C(═O)— | —C(═O)— —C(═O)— —C(═O)— —C(═O)— —C(═O)— —C(═O)— —C(═S)— —C(═S)— —C(═S)— —C(═S)— —C(═SO)— —C(═S)— | 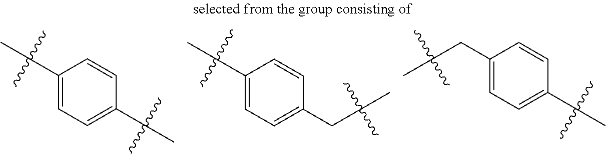 [SMILES: *c1ccc(*)cc1 ; *Cc1ccc(*)cc1 ; *Cc1ccc(*)cc1] 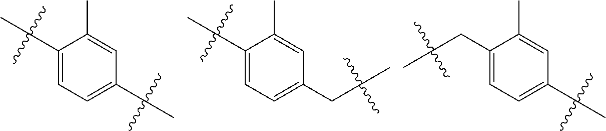 |
| | | 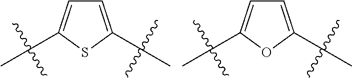 [SMILES: *c1ccc(*)s1 ; *c1ccc(*)o1] |

In the following especially preferred embodiments of the present invention are described:
1. Use of a compound a salt or solvate thereof as C4 plant selective herbicide wherein said compound comprises a cyclic alkyl, aryl, heterocycloalkyl or heteroaryl group, and wherein said compound further comprises
   (a) at least a functional group Z being —C(═Y^{Z})R¹, wherein Y^{Z} is selected from the group consisting of O, NH and S, preferably wherein Y^{Z} is O and wherein R¹ is selected from the group consisting of H, OH, —O—R², —S—R² and —NR²R³, wherein R² and R³, are independently of each other, selected from the group consisting of H, alkyl, cycloalkyl, heterocycloalkyl, aryl and heteroaryl,
      and a functional group Q which is —C(═Y^{Q})R⁴, wherein Y^{Q} is selected from the group consisting of O, NH and S, preferably wherein Y^{Q} is O and wherein R⁴ is selected from the group consisting of H, OH, —O—R^{4#}, —S—R^{4#} and NR^{4#}R^{5#}, wherein R^{4#} and R^{5#}, are independently of each other, selected from the group consisting of H, alkyl, cycloalkyl, heterocycloalkyl, aryl and heteroaryl,
   or
   (b) at least one electron donating group selected from the group consisting of —OH, —SH, —O—R^{p}, —S—R^{p}, —NR^{p}R^{q} and —O—C(═Y^{p})R^{r}, wherein R^{p} and R^{q} are, independently of each other, selected from the group consisting of H, alkyl, cycloalkyl, heterocycloalkyl, aryl and heteroaryl, and wherein R^{r} is —OH, —NH₂, —NH-Alkyl, —O-Alkyl, or —S-Alkyl, and wherein Y^{p} is selected from the group consisting of —S—, —O—, —NH,
   said compound being capable of binding to the malate binding site comprised by a phosphoenolpyruvate carboxylase from a C4 plant, thereby inhibiting said phosphoenolpyruvate carboxylase,
   and wherein the cyclic alkyl, aryl, heterocycloalkyl or heteroaryl group inhibits binding to the malate binding site of a phosphoenolpyruvate carboxylase from a C3 plant.
2. Use according to embodiment 1, wherein the compound has a structure according to the formula (I)

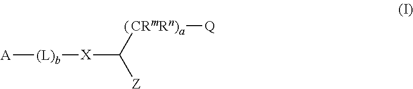

wherein A is the cyclic alkyl (cycloalkyl), aryl, heterocycloalkyl, or heteroaryl group and wherein A is an at least bicyclic alkyl, aryl, heterocycloalkyl, or heteroaryl group preferably a bicyclic aryl or heteroalkyl group,
X is a functional group,
preferably X is —Y^{X}—, —C(═Y^{XX})—, wherein Y^{X} is selected from the group consisting of —S—, —O—, —NH—, —N(CH₃)— and NH—NH—,
and Y^{XX} is selected from the group consisting of —S—, —O—, —NH
and wherein in case X is —Y^{X}—, F′ is —C(═Y^{F})— or —C(═Y^{F}) and wherein in case X is —C(═Y^{X})—, —C(═Y^{X})—NR^{X}—, F¹ is Y^{F1}—,
in particular —NH— or —C(═Y)— with Y being S or O,
L is a linking moiety, preferably a linking moiety comprising an aryl group,
integer b is 0 or 1,
integer a is in the range of from 2 to 5
and wherein R^{m} and Rⁿ are, independently of each other, H or alkyl, preferably H or methyl, in particular H.
3. Use according to embodiment 2, wherein Q is COOH, the compound having the structure (II):

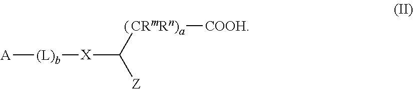

4. Use according to embodiment 2, wherein Z is COOH, the compound having the structure:

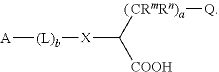

5. Use according to any of embodiments to 2 to 4, wherein Z is COOH, the compound having the structure (III):

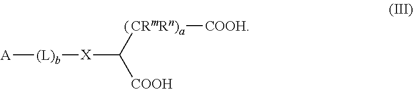

6. Use according to any of embodiments to 2 to 5, wherein Z is COOH, the compound having the structure (III):

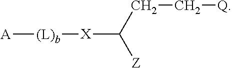

7. Use according to any of embodiments 2 to 6, wherein L has a structure according to the following formula

-[F²]_{c}-[L¹]_{d}-[F¹]ₑ-
wherein L¹ is a linking moiety, preferably selected from the group consisting of alkyl, alkenyl, alkylaryl, arylalkyl, aryl, heteroaryl, alkylheteroaryl and hetero arylalkyl,
and wherein integer d is 0 or 1,
and wherein
F¹ is a functional group consisting of —C(═Y^{F})—, —C(═Y^{F})—NR^{Y1},
wherein Y^{F1} is selected from the group consisting of —S—, —O—, —NH—, —N(CH₃)— and NH—NH—,
Y^{F} is selected from the group consisting of —S—, —O—, —NH
and R^{Y1} is H or alkyl,
and wherein in case X is —Y^{X}—, F¹ is —C(═Y^{F})— or —C(═Y^{F})—NR^{Y1} and wherein in case X is —C(═Y^{X})—, —C(═Y^{XX})—NR^{X}—, F¹ is —Y^{F1}—,
integer e is 0 or 1,
F² is a functional group selected from the group consisting of —Y^{F2}, —C(═Y^{FF})—, —C(═Y^{FF})—NR^{Y2}— and —NR^{Y2}—C(═Y^{FF})—
wherein Y^{F2} is selected from the group consisting of —S—, —O—, —NH—, —N(CH₃)—NH—NH—,
YF^{F} is selected from the group consisting of —S—, —O—, —NH
and R^{Y2} is H or alkyl,
and wherein integer c is 0 or 1.
8. Use according to embodiment 7, wherein X is NH— and wherein L is —Y^{F2}-L¹-C(═Y^{F})—
   wherein L¹ wherein L¹ is alkylaryl, arylalkyl, heteroaryl or aryl group, preferably a group having one of the following structures:

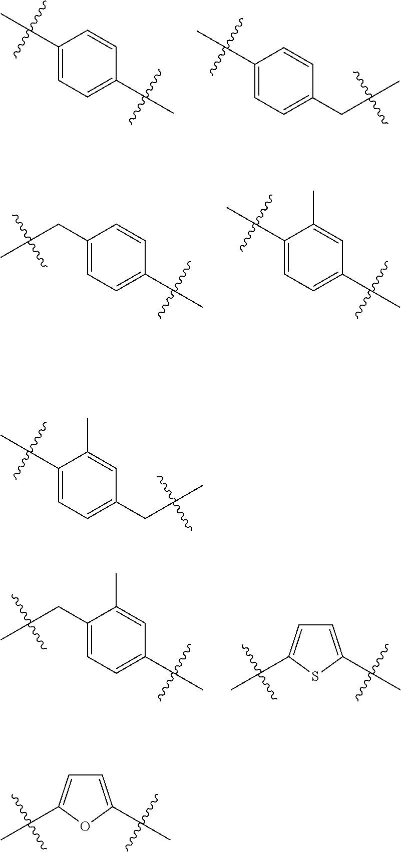

more preferably wherein L is selected from the following structures:

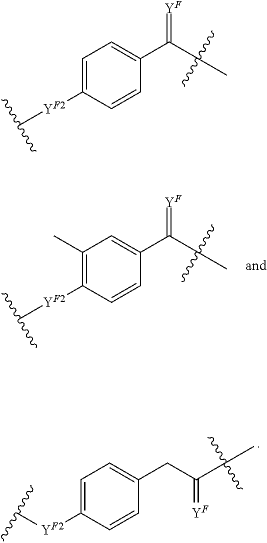

9. Use according to any of embodiments 2 to 8, wherein A comprises one of the following core structures:

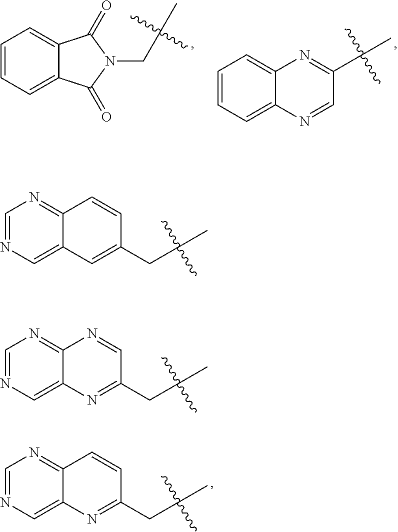 [SMILES: *CN1C(=O)c2ccccc2C1=O ; *c1cnc2ccccc2n1 ; *Cc1ccc2ncncc2c1 ; *Cc1cnc2ncncc2n1 ; *Cc1ccc2ncncc2n1]

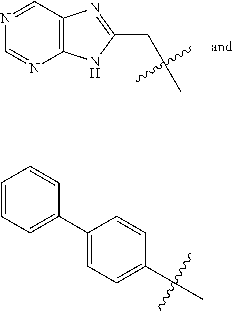 [SMILES: *Cc1nc2cncnc2[nH]1 ; *c1ccc(-c2ccccc2)cc1]

or the core structure

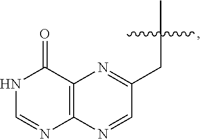 [SMILES: *Cc1cnc2nc[nH]c(=O)c2n1]

in particular, wherein A has a structure selected from the group consisting of:

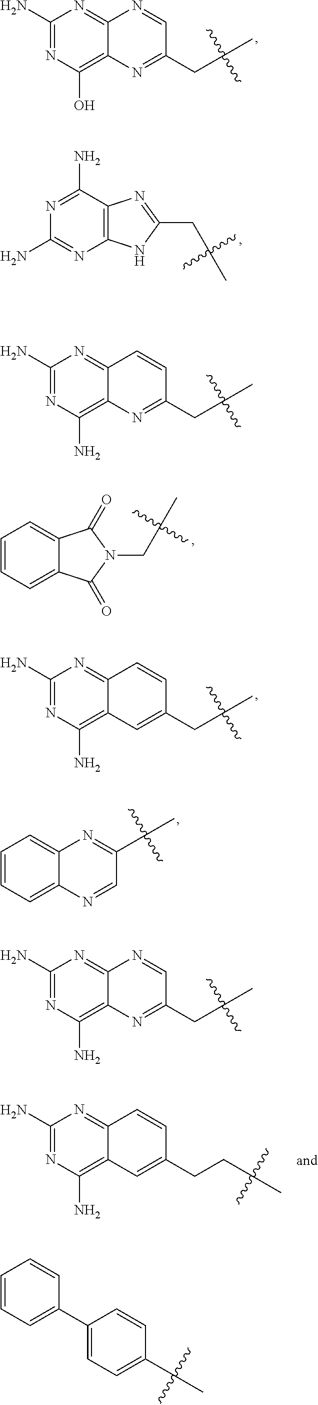

or the structure

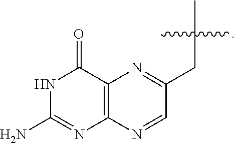 [SMILES: *Cc1cnc2nc(N)[nH]c(=O)c2n1]

more preferably, of the group consisting of

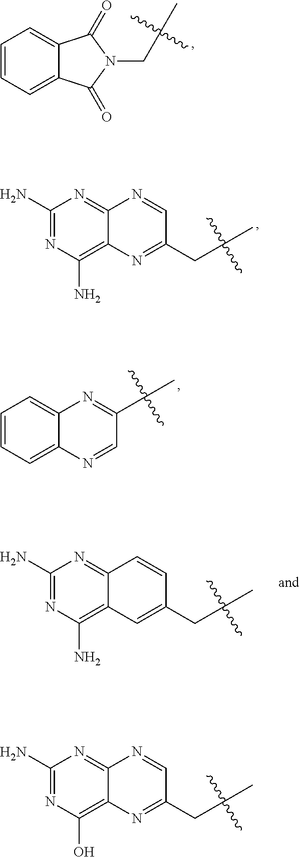 [SMILES: *CN1C(=O)c2ccccc2C1=O ; *Cc1cnc2nc(N)nc(N)c2n1 ; *c1cnc2ccccc2n1 ; *Cc1ccc2nc(N)nc(N)c2c1 ; *Cc1cnc2nc(N)nc(O)c2n1]

or the structure

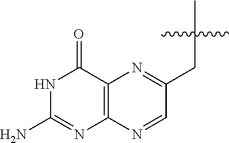 [SMILES: *Cc1cnc2nc(N)[nH]c(=O)c2n1]

10. Use according to any of embodiments 2 to 9, wherein A is a stereoid comprising the core structure

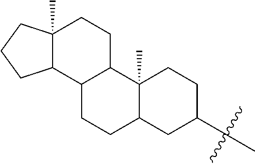 [SMILES: *C1CC[C@@]2(C)C(CCC3C4CCC[C@@]4(C)CCC32)C1]

preferably comprising the core structure

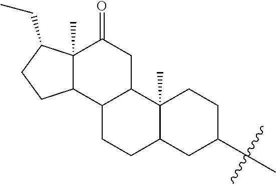 [SMILES: *C1CC[C@@]2(C)C(CCC3C2CC(=O)[C@@]2(C)C3CC[C@@H]2CC)C1]

more preferably wherein A has the structure

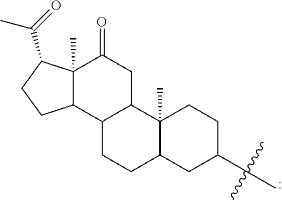 [SMILES: *C1CC[C@@]2(C)C(CCC3C2CC(=O)[C@@]2(C)C3CC[C@@H]2C(C)=O)C1]

By way of example, the following preferred compounds of the invention are described:

TABLE 2
Preferred compounds of the invention

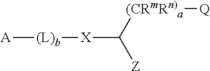

| A | (L)b | X | Z | (CR^{m}Rⁿ)ₐ | Q |
|---|---|---|---|---|---|
| 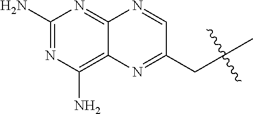 [SMILES: *Cc1cnc2nc(N)nc(N)c2n1] | 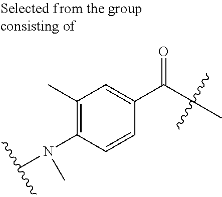 [SMILES: *C(=O)c1ccc(N(*)C)c(C)c1] | —NH— | —COOH | —CH₂—CH₂— | —COOH |
| | 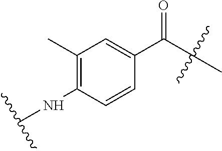 [SMILES: *Nc1ccc(C(*)=O)cc1C] | | | | |
| | 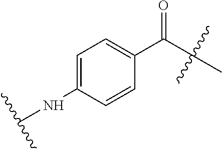 [SMILES: *Nc1ccc(C(*)=O)cc1] | | | | |
| | 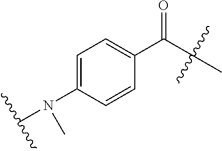 [SMILES: *C(=O)c1ccc(N(*)C)cc1] | | | | |
| | 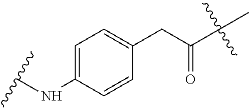 [SMILES: *Nc1ccc(CC(*)=O)cc1] | | | | |
| | 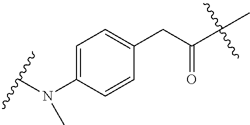 [SMILES: *C(=O)Cc1ccc(N(*)C)cc1] | | | | |
| 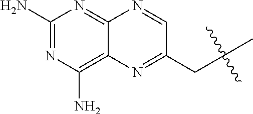 [SMILES: *Cc1cnc2nc(N)nc(N)c2n1] | 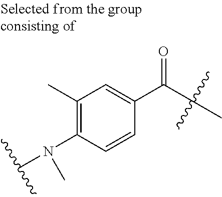 [SMILES: *C(=O)c1ccc(N(*)C)c(C)c1] | —NH— | —COOH | —CH₂— | —COOH |
| | 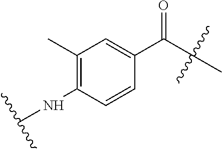 [SMILES: *Nc1ccc(C(*)=O)cc1C] | | | | |
| | 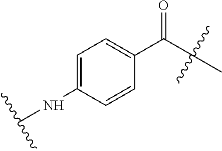 [SMILES: *Nc1ccc(C(*)=O)cc1] | | | | |
| | 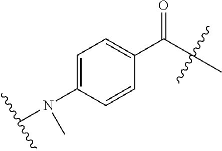 [SMILES: *C(=O)c1ccc(N(*)C)cc1] | | | | |
| | 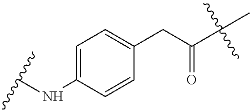 [SMILES: *Nc1ccc(CC(*)=O)cc1] | | | | |
| | 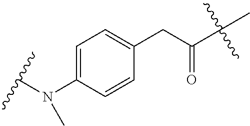 [SMILES: *C(=O)Cc1ccc(N(*)C)cc1] | | | | |
| 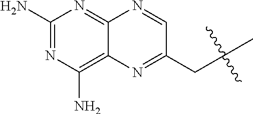 [SMILES: *Cc1cnc2nc(N)nc(N)c2n1] | 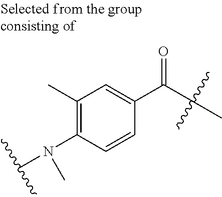 [SMILES: *C(=O)c1ccc(N(*)C)c(C)c1] | —N(Me)— | —COOH | —CH₂—CH₂— | —COOH |
| | 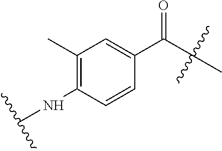 [SMILES: *Nc1ccc(C(*)=O)cc1C] | | | | |
| | 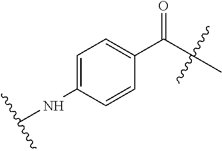 [SMILES: *Nc1ccc(C(*)=O)cc1] | | | | |
| | 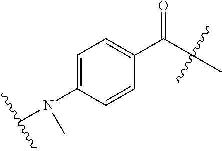 [SMILES: *C(=O)c1ccc(N(*)C)cc1] | | | | |
| | 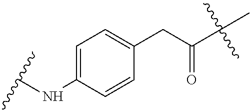 [SMILES: *Nc1ccc(CC(*)=O)cc1] | | | | |
| | 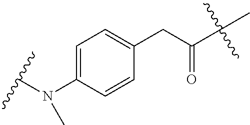 [SMILES: *C(=O)Cc1ccc(N(*)C)cc1] | | | | |
| 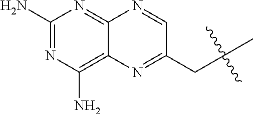 [SMILES: *Cc1cnc2nc(N)nc(N)c2n1] | 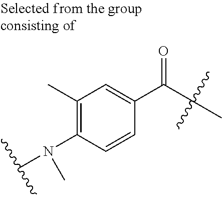 [SMILES: *C(=O)c1ccc(N(*)C)c(C)c1] | —N(Me)— | —COOH | —CH₂- - - - | —COOH |
| | 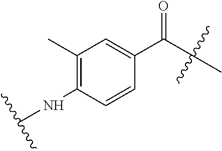 [SMILES: *Nc1ccc(C(*)=O)cc1C] | | | | |
| | 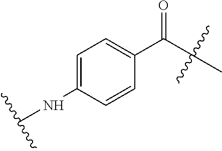 [SMILES: *Nc1ccc(C(*)=O)cc1] | | | | |
| | 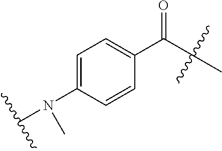 [SMILES: *C(=O)c1ccc(N(*)C)cc1] | | | | |
| | 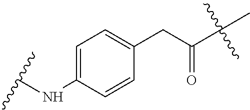 [SMILES: *Nc1ccc(CC(*)=O)cc1] | | | | |
| | 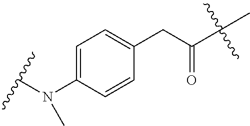 [SMILES: *C(=O)Cc1ccc(N(*)C)cc1] | | | | |
| 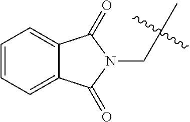 [SMILES: *CN1C(=O)c2ccccc2C1=O] | | —C(═O)—NH | —COOH | —CH₂—CH₂— or —CH₂- - - - | —COOH |
| 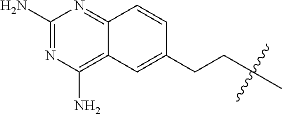 [SMILES: *CCc1ccc2nc(N)nc(N)c2c1] | 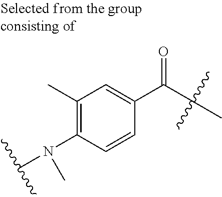 [SMILES: *C(=O)c1ccc(N(*)C)c(C)c1] | —NH— | —COOH | —CH₂—CH₂— | —COOH |
| | 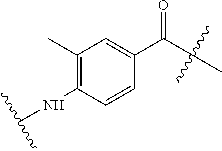 [SMILES: *Nc1ccc(C(*)=O)cc1C] | | | | |
| | 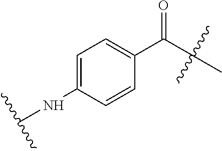 [SMILES: *Nc1ccc(C(*)=O)cc1] | | | | |
| | 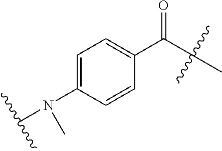 [SMILES: *C(=O)c1ccc(N(*)C)cc1] | | | | |
| | 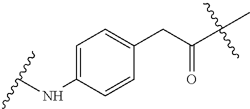 [SMILES: *Nc1ccc(CC(*)=O)cc1] | | | | |
| | 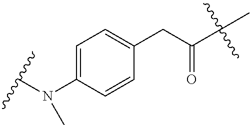 [SMILES: *C(=O)Cc1ccc(N(*)C)cc1] | | | | |
| 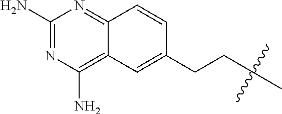 [SMILES: *CCc1ccc2nc(N)nc(N)c2c1] | 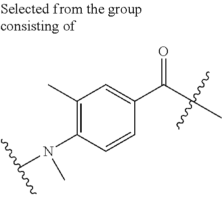 [SMILES: *C(=O)c1ccc(N(*)C)c(C)c1] | —NH— | —COOH | —CH₂— | —COOH |
| | 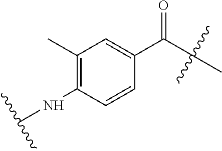 [SMILES: *Nc1ccc(C(*)=O)cc1C] | | | | |
| | 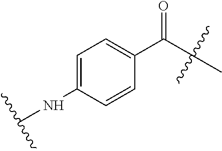 [SMILES: *Nc1ccc(C(*)=O)cc1] | | | | |
| | 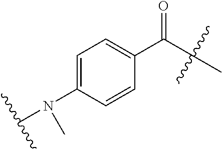 [SMILES: *C(=O)c1ccc(N(*)C)cc1] | | | | |
| | 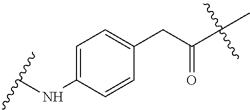 [SMILES: *Nc1ccc(CC(*)=O)cc1] | | | | |
| | 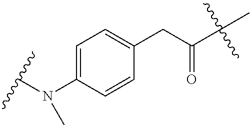 [SMILES: *C(=O)Cc1ccc(N(*)C)cc1] | | | | |
| 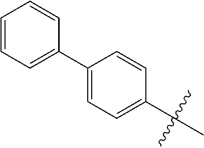 [SMILES: *c1ccc(-c2ccccc2)cc1] | b = 0 | —C(═O)—NH | —COOH | —CH₂— Or —CH₂—CH₂— | —COOH |
| 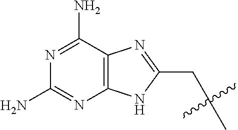 [SMILES: *Cc1nc2c(N)nc(N)nc2[nH]1] | 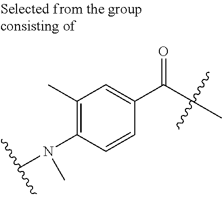 [SMILES: *C(=O)c1ccc(N(*)C)c(C)c1] | —C(═O)—NH | —COOH | —CH₂— or —CH₂—CH₂— | —COOH |
| | 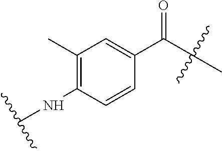 [SMILES: *Nc1ccc(C(*)=O)cc1C] | | | | |
| | 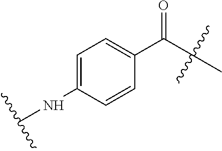 [SMILES: *Nc1ccc(C(*)=O)cc1] | | | | |
| | 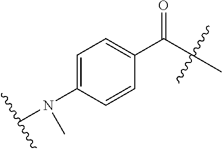 [SMILES: *C(=O)c1ccc(N(*)C)cc1] | | | | |
| | 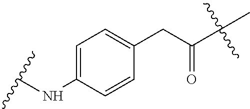 [SMILES: *Nc1ccc(CC(*)=O)cc1] | | | | |
| | 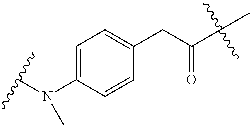 [SMILES: *C(=O)Cc1ccc(N(*)C)cc1] | | | | |
| 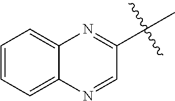 [SMILES: *c1cnc2ccccc2n1] | 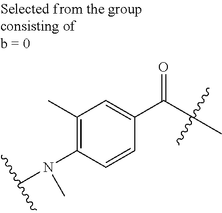 [SMILES: *C(=O)c1ccc(N(*)C)c(C)c1] | —C(═O)—NH | —COOH | —CH₂— or —CH₂—CH₂ | —COOH |
| | 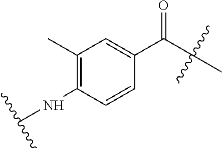 [SMILES: *Nc1ccc(C(*)=O)cc1C] | | | | |
| | 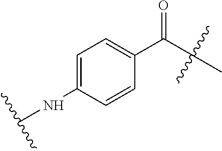 [SMILES: *Nc1ccc(C(*)=O)cc1] | | | | |
| | 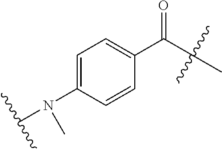 [SMILES: *C(=O)c1ccc(N(*)C)cc1] | | | | |
| | 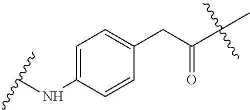 [SMILES: *Nc1ccc(CC(*)=O)cc1] | | | | |
| | 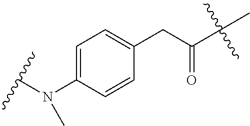 [SMILES: *C(=O)Cc1ccc(N(*)C)cc1] | | | | |
| 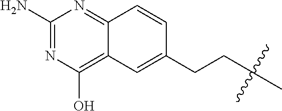 [SMILES: *CCc1ccc2nc(N)nc(O)c2c1] | 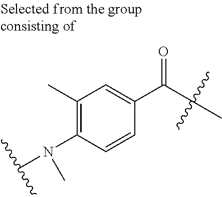 [SMILES: *C(=O)c1ccc(N(*)C)c(C)c1] | C(═O)—NH | —COOH | —CH₂—CH₂— or —CH₂- - - - | —COOH |
| | 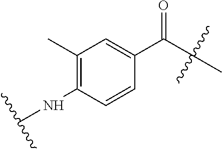 [SMILES: *Nc1ccc(C(*)=O)cc1C] | | | | |
| | 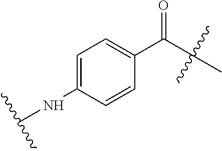 [SMILES: *Nc1ccc(C(*)=O)cc1] | | | | |
| | 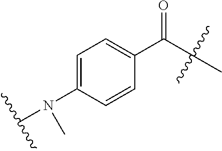 [SMILES: *C(=O)c1ccc(N(*)C)cc1] | | | | |
| | 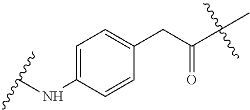 [SMILES: *Nc1ccc(CC(*)=O)cc1] | | | | |
| | 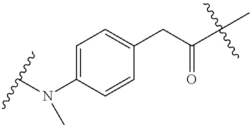 [SMILES: *C(=O)Cc1ccc(N(*)C)cc1] | | | | |
| 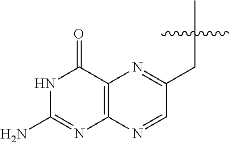 [SMILES: *Cc1cnc2nc(N)[nH]c(=O)c2n1] | 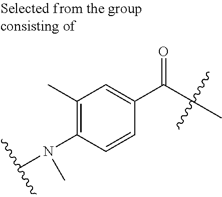 [SMILES: *C(=O)c1ccc(N(*)C)c(C)c1] | —NH— | —COOH | —CH₂—CH₂— | —COOH |
| | 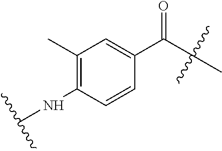 [SMILES: *Nc1ccc(C(*)=O)cc1C] | | | | |
| | 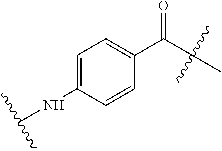 [SMILES: *Nc1ccc(C(*)=O)cc1] | | | | |
| | 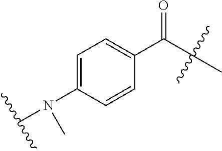 [SMILES: *C(=O)c1ccc(N(*)C)cc1] | | | | |
| | 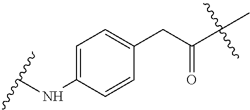 [SMILES: *Nc1ccc(CC(*)=O)cc1] | | | | |
| | 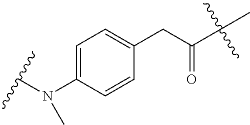 [SMILES: *C(=O)Cc1ccc(N(*)C)cc1] | | | | |

According to an alternative embodiment of the present invention, the compound has a structure of the formula (IVa) or (IVb)

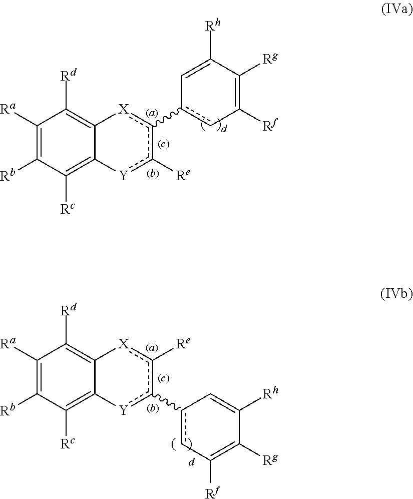

wherein
X is selected from the group consisting of —C(═O)—, —O— and —N—,
Y is selected from the group consisting of —C(H)ₚ—, —O— and —N—,
with p being 1 or 2,
the bond (a) is a single or double bond,
the bond (b) is a single or double bond,
the bond (c) is a single or double bond,
and wherein R^{a}, R^{b}, R^{c}, R^{d}, R^{e}, R^{f}, R^{g} and R^{h} are, independently of each other, selected from the group consisting of H, Alkyl and an electron donating group, or R^{a}, R^{b}, R^{c}, R^{d}, R^{e}, R^{f}, R^{g} and R^{h} independently of each other, selected from the group consisting of H, Alkyl, —C(═Y^{p})R^{r} and an electron donating group, wherein R^{r} is —OH, —NH₂, —NH-Alkyl, —O-Alkyl, or —S-Alkyl, and wherein Y^{p} is selected from the group consisting of —S—, —O—, —NH, preferably R^{a}, R^{b}, R^{c}, R^{d}, R^{e}, R^{f}, R^{g} and R^{h} independently of each other, selected from the group consisting of H, Alkyl, —C(═Y^{p})R^{r} and an electron donating group, in particular R^{a}, R^{b}, R^{c}, R^{d}, R^{e}, R^{f}, R^{g} and R^{h} are, independently of each other, selected from the group consisting of H, Alkyl and an electron donating group, wherein at least one of R^{a}, R^{h}, R^{e}, R^{d}, R^{e} and R^{f} is an electron donating group, with the proviso that in case the bond (c) is a double bond (a) and (b) are both single bonds, and in case X is —C(═O)—, the bond (a) is a single bond, and wherein in case Y is —C(═O)—, the bond (b) is a single bond.

The term “electron donating group” is recognized in the art, and denotes the tendency of a functional group to donate valence electrons to neighboring atoms by means of a difference in electronegativity with respect to the neighbouring atom (inductive effect) and/or by donating of pi-electrons via conjugation (mesomeric effect). According to the invention said electron donating group is preferably selected from the group consisting of —OH, —SH, —O—R^{p}, —S—R^{p}, —NR^{p}R^{q} and —OC(═Y^{p})R^{r}, wherein R^{p} and R^{q} are, independently of each other, selected from the group consisting of H, alkyl, cycloalkyl, heterocycloalkyl, aryl and heteroaryl, R^{r} is preferably selected from —OH, —NH₂, —NH-Alkyl, —O-Alkyl, or —S-Alkyl, and wherein Y^{p} is selected from the group consisting of —S—, —O—, —NH, more preferably the electron donating group is selected from the group consisting of —OH, —SH, —O—R^{p}, —S—R^{p} and —NR^{p}R^{q}, wherein R^{p} and R^{q} are, independently of each other, selected from the group consisting of H, alkyl, cycloalkyl, heterocycloalkyl, aryl and heteroaryl, R^{p} and R^{q} are, independently of each other, preferably selected from the group consisting of H or alkyl or cycloalkyl, more preferably H or a linear or branched alkyl group or a cycloalkyl group having from 1 to 10 carbon atoms, preferably an alkyl group or cycloalkyl group selected from the group consisting of methyl, ethyl, propyl, pentyl, butyl, cyclopropyl, cyclobutyl, cyclopropenyl, cyclobutenyl, and hexyl, more preferably H or methyl or ethyl. More preferably at least one of R^{p} and R^{q} is H and one of R^{p} and R^{q} is H or an alkyl group, preferably H or a linear or branched alkyl group having from 1 to 10 carbon atoms, preferably H or an alkyl group selected from the group consisting of methyl, ethyl, propyl, pentyl, butyl and hexyl, more preferably methyl or ethyl.

R^{r} is preferably selected from the group consisting of —OH, —NH₂, —NH-Alkyl, —O-Alkyl and SH-Alkyl. Y^{p} is preferably selected from the group consisting of —S—, —O—, —NH,

Preferably the at least one electron donation group is —OH, i.e. at least one of R^{a}, R^{b}, R^{c}, R^{d}, R^{e} and R^{f} is —OH. Thus, the present invention to the use of a compound, as described above, wherein the compound has the structure (IVa) or (IVb), wherein at least one of R^{a}, R^{b}, R^{c}, R^{d}, R^{e} and R^{f} is —OH.

In the above-mentioned formulas (IVa) and (IVb), the bond “ [symbol] ” represents a bond with non-defined stereochemistry, i.e. this term represents a bond encompassing both possible stereochemistries, i.e. atropisomers. Atropisomers are stereoisomers resulting from hindered rotation around this single bond.

The compounds may thus be a mixture of atropisomers or the isolated form of one specific stereoisomer. Preferably, the compound of the invention is used as isolated stereoisomer.

### The Groups X and Y

As described above, the group X in the above-mentioned formulas (IVa) and (IVb) is selected from the group consisting of —C(═O)—, —O— and —N—, preferably X is O or N.

Thus, the compound has preferably one of the following structures:

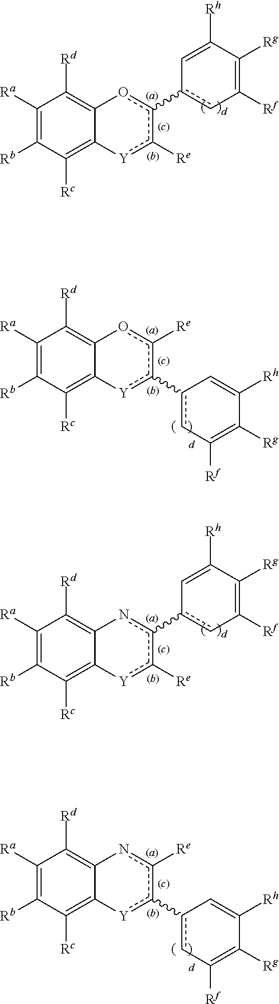

hi case X is O, (a) and (b) are preferably single bonds, whereas (c) is a single or a double bond.

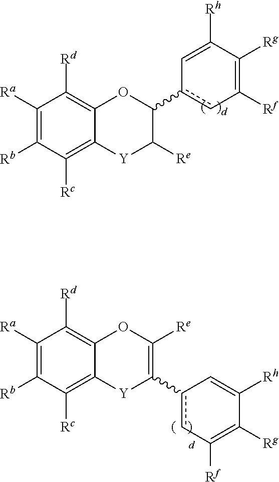

In case X is O, Y is preferably —C(H)ₚ— or —C(═O)—, the compound thus preferably having a structure according to one of the following formulas:

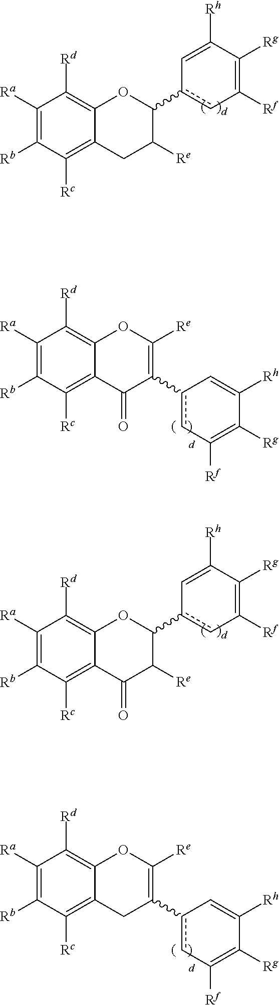

According to an alternative preferred embodiment, X is O, (a) and (b) are double bonds:

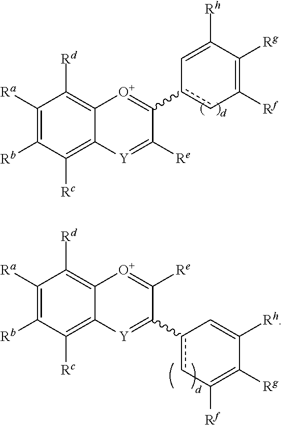

In case X is N, (a) and (b) are preferably double bonds, the compound thus having one of the following structures:

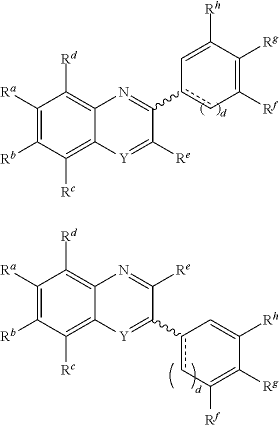

According to this embodiment, Y is most preferably —N—, the compound thus most preferably having a structure according to the following fog hulas:

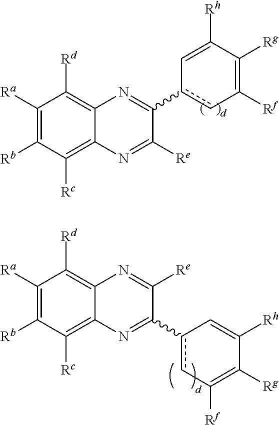

In the structural units

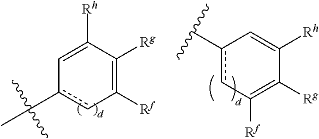

integer d is 0 or 1. In case d is 1, the core structure of the structure shown above is a 6-membered aromatic ring, being substituted with groups R^{h}, R^{g} and R^{f}.

In case (d) is O, the core structure of the structure shown above is a 5-membered aromatic ring, being substituted with groups R^{h}, R^{g} and R^{f}.

According to a particularly preferred embodiment of the invention d is 1. More preferably, the compound has a structure selected from the group consisting of

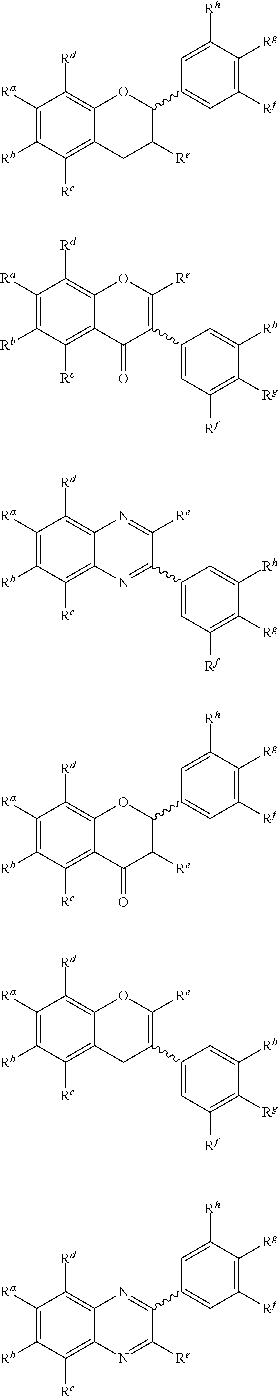

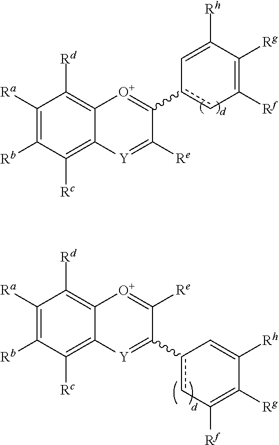

more preferably a structure selected from the group consisting of

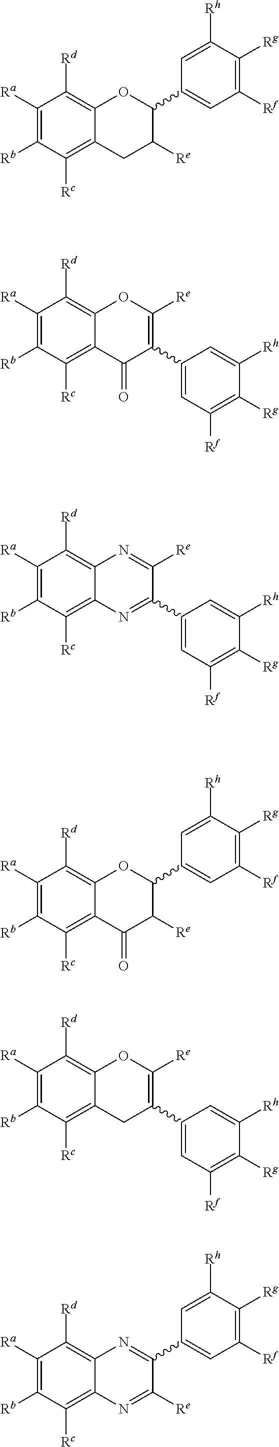

Most preferably, R^{a}, R^{b}, R^{c}, R^{d}, R^{e}, R^{f}, le and R^{h} are, independently of each other, selected from the group consisting of H, —O-Methyl, —OH and Methyl.

Thus, the present invention also relates to compound, as described above wherein the compound has a structure selected from the group consisting of

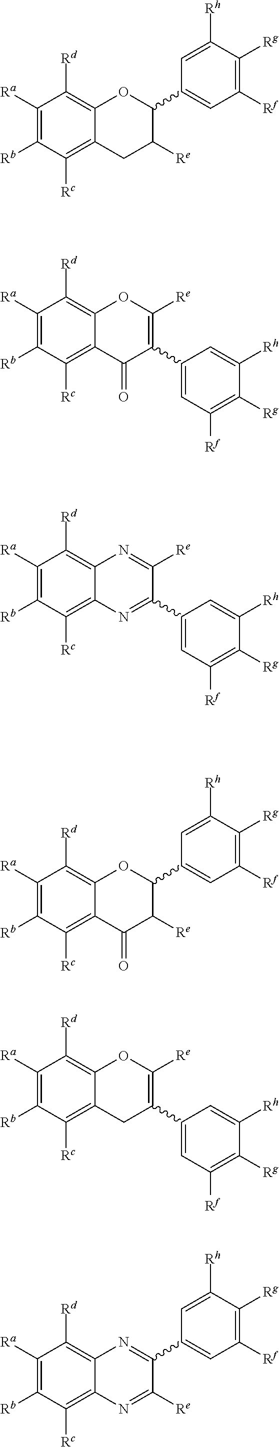

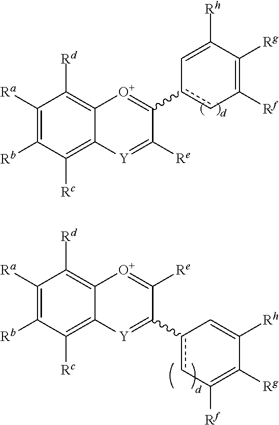

and wherein R^{a}, R^{b}, R^{c}, R^{d}, R^{e}, R^{f}, R^{g} and R^{h} are, independently of each other, selected from the group consisting of H, —O-Methyl, —OH and Methyl, or are, independently of each other, selected from the group consisting of H, —O-Methyl, —OH. —NH₂, —COOH and Methyl.

According to a further embodiment of the present invention, the compound has a structure of the formula (IVa) or (IVb)

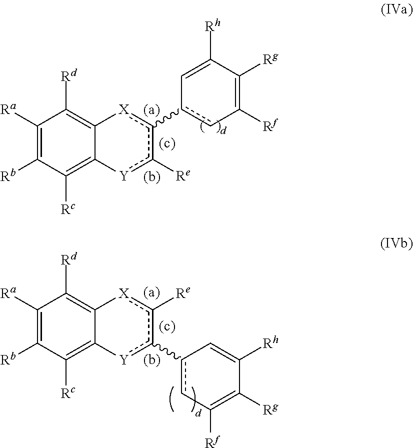

wherein
X is selected from the group consisting of —C(═O)—, —O— and —N—,
Y is selected from the group consisting of —C(H)ₚ—, —C(═O)—, —O— and —N—,
with p being 1 or 2,
the bond (a) is a single or double bond,
the bond (b) is a single or double bond,
the bond (c) is a single or double bond,
and wherein R^{a} and R^{b} are, independently of each other selected from the group consisting of H, Alkyl, halogen and an electron donating group or R^{a} and R^{b} are, independently of each other selected from the group consisting of H, Alkyl, halogene, —C(═Y^{p})R^{r}, and an electron donating group, wherein R′ is —OH, —NH₂, —NH-Alkyl, —O-Alkyl, or —S-Alkyl, and wherein Y^{p} is selected from the group consisting of —S—, —O—, —NH, or wherein R^{a} and R form together a cycloalkyl, cycloheteroalkyl, aryl or heteroaryl, wherein the cycloalkyl, cycloheteroalkyl, aryl or heteroaryl is a single-ring group or a multicyclic group, and wherein R^{c}, R^{d}, R^{f}, R^{g} and R^{h} are, independently of each other, selected from the group consisting of H, halogene, alkyl and an electron donating group, or wherein R^{c}, R^{d}, R^{f}, R^{g} and R^{h} are, independently of each other, selected from the group consisting of H, Alkyl, halogene, —C(═Y^{p})R^{r}, and an electron donating group, wherein R^{r} is —OH, —NH₂, —NH-Alkyl, —O-Alkyl, or —S-Alkyl, and wherein Y^{p} is selected from the group consisting of —S—, —O—, —NH
and wherein R^{e} is selected from the group consisting of H, Alkyl, halogen, electron donating group, aryl, heteroaryl group, —C(═Y^{p})R^{r} and —O—C(═O)—R*, wherein R* is an aryl or heteroaryl group, wherein at least one of R^{a}, R^{b}, R^{c}, R^{d}, R^{f}, R^{g}, R^{e}, and R^{h} is an electron donating group, with the proviso that in case the bond (c) is a double bond (a) and (b) are both single bonds, and in case X is —C(═O)—, the bond (a) is a single bond, and wherein in case Y is —C(═O)—, the bond (b) is a single bond. Preferably, the electron donating group is selected from the group consisting of —OH, —SH, —O—R^{p}, —S—R^{p} and —NR^{p}R^{q}, wherein R^{p} and R^{q} are, independently of each other, selected from the group consisting of H, alkyl, cycloalkyl, heterocycloalkyl, aryl and heteroaryl, preferably H or alkyl or cycloalkyl, more preferably H or a linear or branched alkyl group or a cycloalkyl group having from 1 to 10 carbon atoms, preferably an alkyl group or cycloalkyl group selected from the group consisting of methyl, ethyl, propyl, pentyl, butyl, cyclopropyl, cyclobutyl, cyclopropenyl, cyclobutenyl, and hexyl, more preferably H or methyl or ethyl. More preferably at least one of R^{p} and R^{q} is H and one of R^{p} and R^{q} is H or an alkyl group, preferably H or a linear or branched alkyl group having from 1 to 10 carbon atoms, preferably H or an alkyl group selected from the group consisting of methyl, ethyl, propyl, pentyl, butyl and hexyl, more preferably methyl or ethyl. R^{r} is preferably selected from the group consisting of —OH, —NH₂, —NH-Alkyl, —O-Alkyl or —NH-Alkyl. Y^{p} is preferably selected from the group consisting of —S—, —O—, —NH. Preferably the at least one electron donation group is —OH, i.e. at least one of R^{a}, R^{b}, R^{c}, R^{d}, R^{f}, R^{g}, R^{e}, and R^{h} is —OH. Thus, the present invention also relates to the use of a compound, as described above, wherein the compound has the structure (IVa) or (IVb), wherein at least one of R^{a}, R^{h}, R^{c}, R^{d}, R^{f}, R^{g}, R^{e}, and R^{h} is —OH. As described above, the group X in the above-mentioned formulas (IVa) and (IVb) is selected from the group consisting of —C(═O)—, —O— and —N—, preferably X is O or N.

According to a preferred embodiment, R^{a} and R^{b} form a cycloalkyl, cycloheteroalkyl, aryl or heteroaryl, wherein the cycloalkyl, cycloheteroalkyl, aryl or heteroaryl is a single-ring group or a multicyclic group. It is to be understood that the cycloalkyl, cycloheteroalkyl, aryl or heteroaryl may be substituted or unsubstituted. Preferably, the cycloalkyl, cycloheteroalkyl, aryl or heteroaryl is unsubstituted.

Thus, the present invention also relates to a compound having a structure of the formula (IVa) or (IVb), as described above,

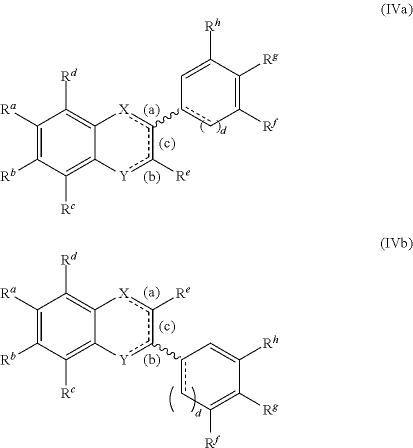

wherein
X is selected from the group consisting of —C(═O)—, —O— and —N—,
Y is selected from the group consisting of —C(H)ₚ—, —C(═O)—, —O— and —N—,
with p being 1 or 2,
the bond (a) is a single or double bond,
the bond (b) is a single or double bond,
the bond (c) is a single or double bond,
and wherein R^{a} and R^{b} form together a cycloalkyl ring, cycloheteroalkyl ring, an aryl ring or a heteroaryl ring, wherein the cycloalkyl, cycloheteroalkyl, aryl or heteroaryl is a single-ring group or a multicyclic group,
and wherein R^{C}, R^{d}, R^{f}, R^{g} and R^{h} are, independently of each other, selected from the group consisting of H, halogene, alkyl and an electron donating group, or wherein R^{c}, R^{d}, R^{f}, R^{g} and R^{h} are, independently of each other, selected from the group consisting of H, Alkyl, halogene, —C(═Y^{p})R^{r}, and an electron donating group, wherein R^{r} is —OH, —NH₂, —NH-Alkyl, —O-Alkyl, or —S-Alkyl, and wherein Y^{p} is selected from the group consisting of —S—, —O—, —NH
and wherein R^{e} is selected from the group consisting of H, Alkyl, halogen, electron donating group, aryl, heteroaryl group, —C(═Y^{p})R^{T} and —O—C(═O)—R*, wherein R* is an aryl or heteroaryl group,
wherein at least one of R^{a}, R^{b}, R^{c}, R^{d}, R^{f}, R^{g}, R^{e}, and R^{h} is an electron donating group, with the proviso that in case the bond (c) is a double bond (a) and (b) are both single bonds, and in case X is —C(═O)—, the bond (a) is a single bond, and wherein in case Y is —C(═O)—, the bond (b) is a single bond. In particular, the present invention also relates to a compound and the use of said compound, as described above, said compound having a structure selected from the group consisting of:

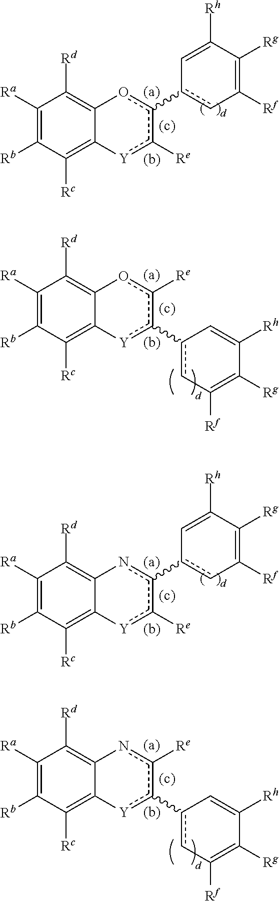

in particular from the group consisting of

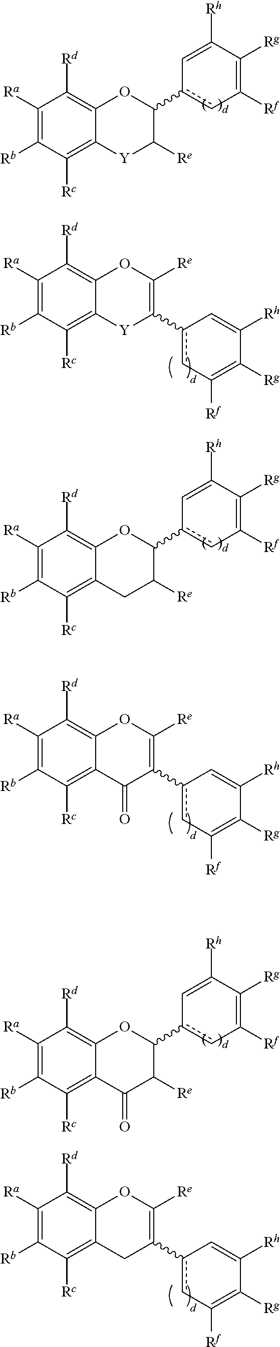

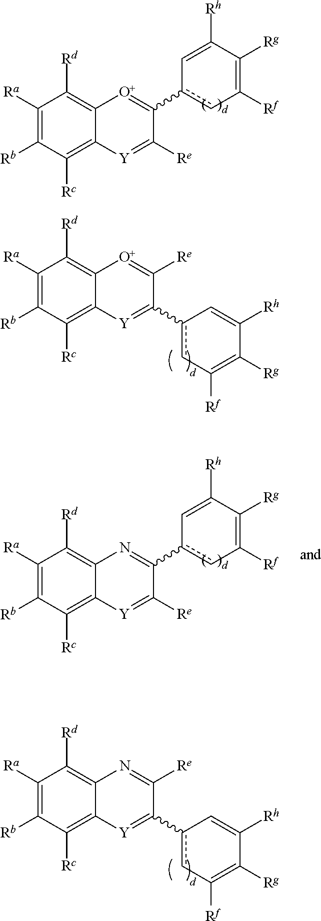

wherein
X is selected from the group consisting of —C(═O)—, —O— and —N—,
Y is selected from the group consisting of —C(H)ₚ—, —C(═O)—, —O— and —N—,
with p being 1 or 2,
and wherein R^{a} and R^{b} form together a cycloalkyl ring, cycloheteroalkyl ring, an aryl ring or a heteroaryl ring, wherein the cycloalkyl, cycloheteroalkyl, aryl or heteroaryl is a single-ring group or a multicyclic group,
and wherein R^{c}, R^{d}, R^{f}, R^{g} and R^{h} are, independently of each other, selected from the group consisting of H, halogene, alkyl and an electron donating group, or wherein R^{c}, R^{d}, R^{f}, R^{g} and R^{h} are, independently of each other, selected from the group consisting of H, Alkyl, halogene, —C(═Y^{p})R^{r}, and an electron donating group, wherein R^{r} is —OH, —NH₂, —NH-Alkyl, —O-Alkyl, or —S-Alkyl, and wherein Y^{p} is selected from the group consisting of S—, —O—, —NH
and wherein R^{e} is selected from the group consisting of H, Alkyl, halogen, electron donating group, aryl, heteroaryl group, —C(═Y^{p})R^{r} and —O—C(═O)—R*, wherein R* is an aryl or heteroaryl group,
wherein at least one of R^{a}, R^{b}, R^{c}, R^{d}, R^{f}, R^{g}, R^{e}, and R^{h} is an electron donating group.

Preferably R^{a} and R^{b} are together —O—CH₂—O— and thus form a 5 membered ring.

By way of example, the following preferred compound is mentioned:

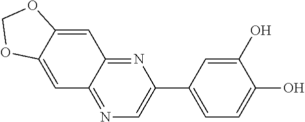 [SMILES: Oc1ccc(-c2cnc3cc4c(cc3n2)OCO4)cc1O]

According to a preferred embodiment, R^{e} is selected from the group consisting of aryl, heteroaryl group and —O—C(═O)—R*, wherein R* is an aryl or heteroaryl group. In particular, R^{e} is a substituted or unsubstituted phenyl group or —O—C(═O)—R* with R* being a substituted or unsubstituted phenyl, preferably wherein R^{e} is a group having the structure

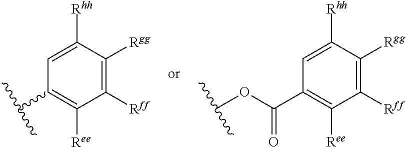

wherein R^{ee}, R^{ff}, R^{gg} and R^{hh} are, independently of each other, selected from the group consisting of H, Alkyl, —C(═Y^{pp})R^{rr}, —O—C(═Y^{pp})R^{rr} and an electron donating group, wherein R^{rr} is —OH, —NH₂, —NH-Alkyl, —O-Alkyl, or —S-Alkyl, and wherein Y^{pp} is selected from the group consisting of —S—, —O—, —NH. More preferably, the compound has a structure selected from the group consisting of

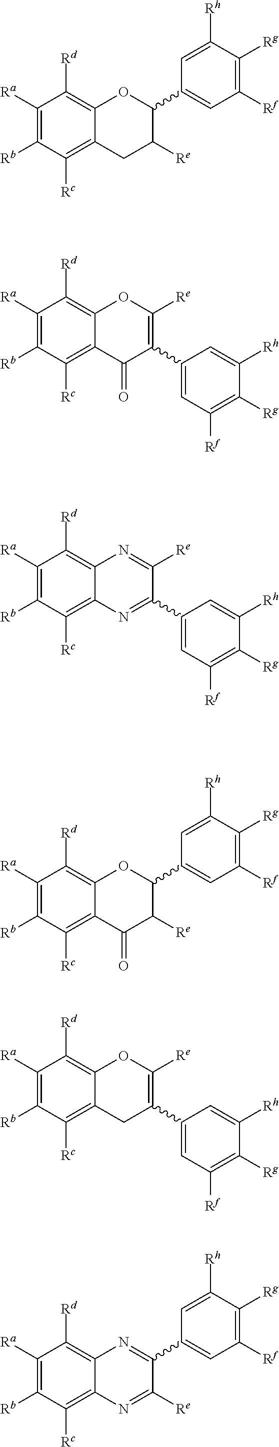

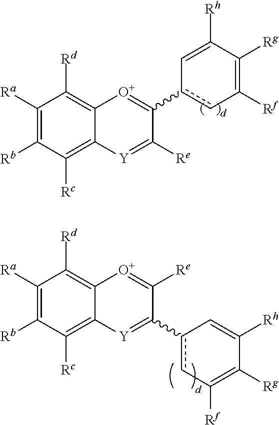

more preferably a structure selected from the group consisting of

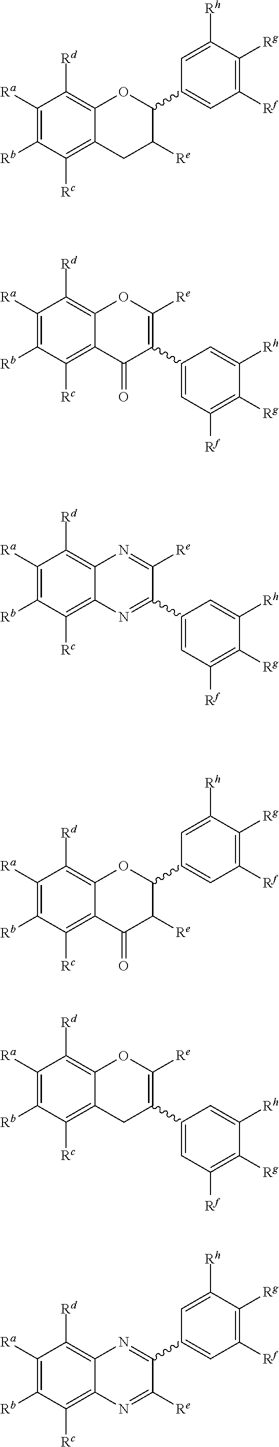

wherein Re is a group having the structure

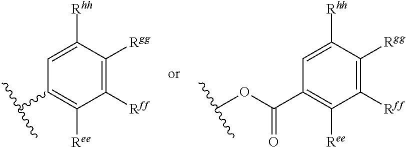

wherein R^{ee}, R^{ff}, R^{gg} and R^{hh} are, independently of each other, selected from the group consisting of H, Alkyl, —C(═Y^{pp})R^{rr}, —O—C(═Y^{pp})R^{rr} and an electron donating group, wherein R^{rr} is —OH, —NH₂, —NH-Alkyl, —O-Alkyl, or —S-Alkyl, and wherein Y^{pp} is selected from the group consisting of —S—, —O—, —NH. More preferably, R^{a}, R^{b}, R^{c}, R^{d}, R^{f}, R^{g} and R^{h} are, independently of each other, selected from the group consisting of H, —O-Methyl, —OH and Methyl, and R^{ee}, R^{ff}, R^{gg} and R^{hh} are, independently of each other, selected from the group consisting of H, —O-Methyl, —OH and Methyl. By way of example, the following preferred compounds are mentioned:

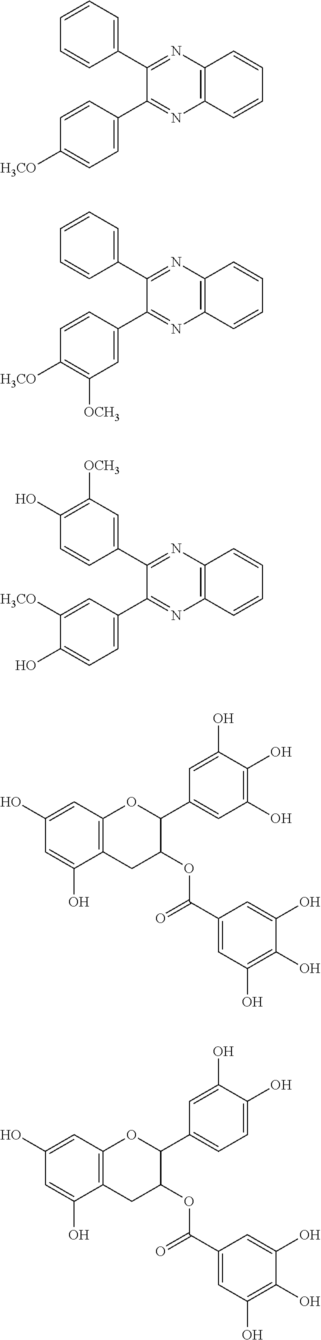

According to a further embodiment of the invention, the compound has a structure of the formula (IV′) or (IV″)

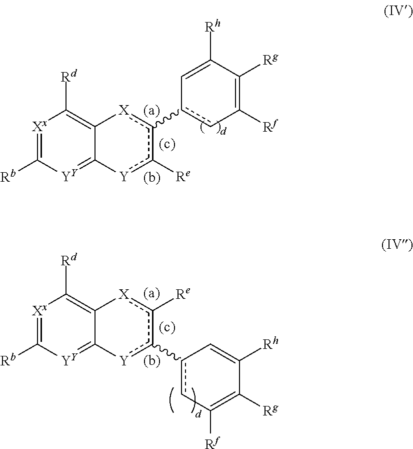

wherein
X is selected from the group consisting of —C(═O)—, —O— and —N—, Y is selected from the group consisting of —C(H)ₚ—, —C(═O)—, —O— and —N—, X^{X} is selected from the group consisting of —C(R^{a})— or N, Y^{y} is selected from the group consisting of —C(R^{c})— or N,
wherein
with p being 1 or 2,
the bond (a) is a single or double bond,
the bond (b) is a single or double bond,
the bond (c) is a single or double bond,
and wherein R^{a} and R^{b} are, independently of each other selected from the group consisting of H, Alkyl, halogen, —C(═Y^{p})R^{r}, and an electron donating group, wherein R^{r} is —OH, —NH₂, —NH-Alkyl, —O-Alkyl, or —S-Alkyl, and wherein Y^{p} is selected from the group consisting of —S—, —O—, —NH,
or wherein R^{a} and R form together a cycloalkyl, cycloheteroalkyl, aryl or heteroaryl, wherein the cycloalkyl, cycloheteroalkyl, aryl or heteroaryl is a single-ring group or a multicyclic group,
and wherein R^{c}, R^{d}, R^{f}, R^{g} and R^{h} are, independently of each other, selected from the group consisting of H, Alkyl, halogene, —C(═Y^{p})R^{r}, and an electron donating group, wherein R^{r} is —OH, —NH₂, —NH-Alkyl, —O-Alkyl, or —S-Alkyl, and wherein Y^{p} is selected from the group consisting of —S—, —O—, —NH,
and wherein R^{e} is selected from the group consisting of H, Alkyl, halogene, —O—C(═Y^{p})R^{r}, electron donating group, aryl, heteroaryl group and —O—C(═O)—R*, wherein R* is an aryl or heteroaryl group, wherein R^{r} is —OH, —NH₂, —NH-Alkyl, —O-Alkyl, or —S-Alkyl, and wherein Y^{p} is selected from the group consisting of —S—, —O—, —NH, and wherein at least one of R^{a}, R^{b}, R^{c}, R^{d}, R^{f}, R^{g}, R^{e}, and R^{h} is an electron donating group, with the proviso that in case the bond (c) is a double bond (a) and (b) are both single bonds, and in case X is —C(═O)—, the bond (a) is a single bond, and wherein in case Y is —C(═O)—, the bond (b) is a single bond. More preferably X^{x} is N and Y^{y} is N, the compound thus having one of the following structures:

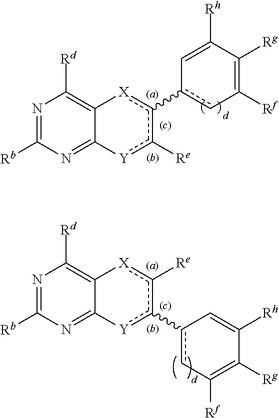

more preferably a structure selected from the group consisting of

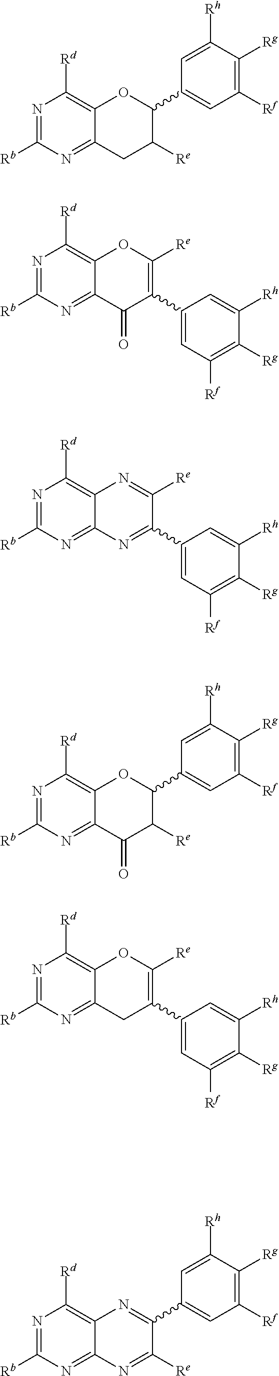

more preferably the structure:

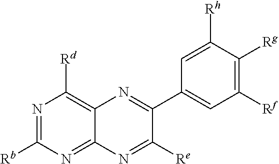

in particular

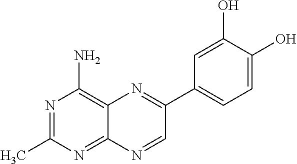 [SMILES: Cc1nc(N)c2nc(-c3ccc(O)c(O)c3)cnc2n1]

In the following especially preferred embodiments of the present invention are described:
1 Use as described above, wherein the compound has a structure of the formula

(IV′) or (IV″)

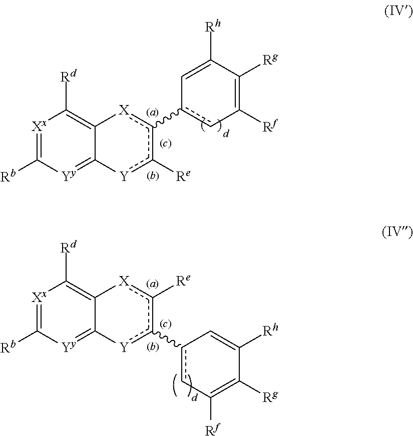

wherein
X is selected from the group consisting of —C(═O)—, —O— and —N—, Y is selected from the group consisting of —C(H)ₚ—, —O— and —N—,
X^{x} is selected from the group consisting of —C(R^{a})— or N, Y^{y} is selected from the group consisting of —C(R^{c})— or N,
with p being 1 or 2,
the bond (a) is a single or double bond,
the bond (b) is a single or double bond,
the bond (c) is a single or double bond,
and wherein R^{a} and R^{b} are, independently of each other selected from the group consisting of H, Alkyl, halogene, —C(═Y^{p})R^{r}, and an electron donating group, wherein R^{r} is —OH, —NH₂, —NH-Alkyl, —O-Alkyl, or —S-Alkyl, and wherein Y^{p} is selected from the group consisting of —S—, —O—, —NH, or wherein R^{a} and R^{b} form together a cycloalkyl, cycloheteroalkyl, aryl or heteroaryl, wherein the cycloalkyl, cycloheteroalkyl, aryl or heteroaryl is a single-ring group or a multicyclic group, and wherein R^{c}, R^{d}, R^{f}, R^{g} and R^{h} are, independently of each other, selected from the group consisting of H, Alkyl, halogene, —C(═Y^{p})R^{r}, and an electron donating group, wherein R^{r} is —OH, —NH₂, —NH-Alkyl, —O-Alkyl, or —S-Alkyl, and wherein Y^{p} is selected from the group consisting of —S—, —O—, —NH,
and wherein R^{e} is selected from the group consisting of H, Alkyl, halogene, —C(═Y^{p})R^{r}, —O—C(═Y^{p})R^{r}, an electron donating group, aryl, heteroaryl group and —O—C(═O)—R*, wherein R* is an aryl or heteroaryl group, and wherein R^{r} is —OH, —NH₂, —NH-Alkyl, —O-Alkyl, or —S-Alkyl, and wherein r is selected from the group consisting of —S—, —O—, —NH
wherein at least one of R^{a}, R^{b}, R^{c}, R^{d}, R^{f}, R^{g}, R^{e} and R^{h} is an electron donating group, with the proviso that in case the bond (c) is a double bond (a) and (b) are both single bonds, and in case X is —C(═O)—, the bond (a) is a single bond, and wherein in case Y is —C(═O)—, the bond (b) is a single bond.
2. The use according to embodiment 1 above, wherein the compound has a structure of the formula (IV′) or (IV″)

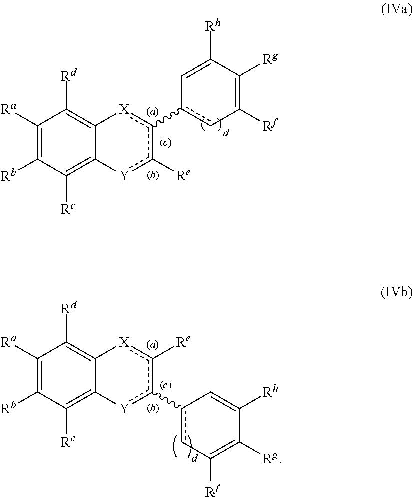

preferably wherein R^{a}, R^{b}, R^{c}, R^{d}, R^{e}, R^{f}, R^{g} and R^{h} are, independently of each other, selected from the group consisting of H, Alkyl and an electron donating group or, independently of each other, selected from the group consisting of H, Alkyl, —C(═Y^{p})R^{r}, and an electron donating group, wherein R^{r} is —OH, —NH₂, —NH-Alkyl, —O-Alkyl, or —S-Alkyl, and wherein Y^{p} is selected from the group consisting of —S—, —O—, —NH,
preferably R^{a}, R^{b}, R^{c}, R^{d}, R^{e}, R^{f}, R^{g} and R^{h} independently of each other, selected from the group consisting of H, Alkyl, —C(═Y^{p})R^{r} and an electron donating group, in particular R^{a}, R^{h}, R^{c}, R^{d}, R^{e}, R^{f}, R^{g} and R^{h} are, independently of each other, selected from the group consisting of H, Alkyl and an electron donating group, wherein R^{p} and R^{q} are, independently of each other H or alkyl,
and wherein R^{r} is —OH, —NH₂, —NH-Alkyl, —O-Alkyl or —NH-Alkyl,
   and wherein Y^{p} is selected from the group consisting of —S—, —O—, —NH, wherein at least one of R^{a}, R^{b}, R^{c}, R^{d}, R^{e} and R^{f} is an electron donating group.
3. Use according to embodiment 1 or 2, wherein at least one of R^{a}, R^{b}, R^{c}, R^{d}, R^{e} and R^{f} is —OH.

TABLE 3
Preferred compounds of the invention, by way of example

| | R^{a} | R^{b} | R^{c} | R^{d} | R^{e} | R^{f} | R^{g} | R^{h} |
|---|---|---|---|---|---|---|---|---|
| | Selected | Selected | Selected | Selected | Selected | Selected | | Selected |
| | from | from | from | from | from | from | | from |
| | the group | the group | the group | the group | the group | the group | Selected from | the group |
| | consisting | consisting | consisting | consisting | consisting | consisting | the group | consisting |
| Structure | of | of | of | of | of | of | consisting of | of |
| 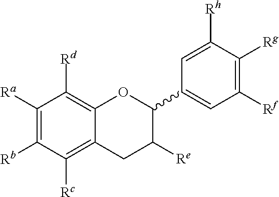 | H, OH | H | H, OH | H | —OH | H, OH | H, OH | H, OH |
| 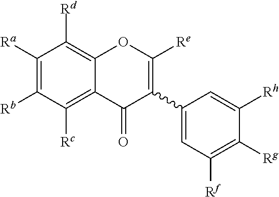 | OH | H, —O—CH₃ | H, OH | H | H | H | OH, —O—CH₃ | H |
| 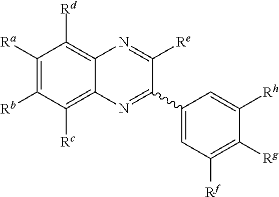 | H, CH_{3:} preferably CH₃ | H, CH₃, preferably CH₃ | H | H | H | OH | OH | H |
| 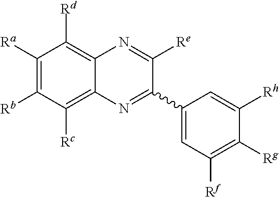 | F | F | H | H | H | OH | OH | OH |
| 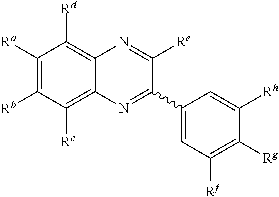 | H | H | H | H | H | H | —O—C(═O)— CH₃ | H |
| 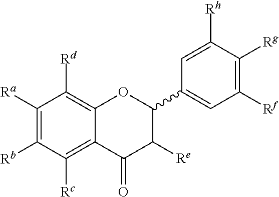 | OH | H | OH | H | H | H | H, OH, —O—CH₃ | H |
| 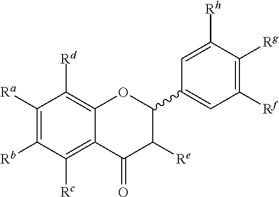 | OH | H | H, OH | H | OH | OH | OH | OH |
| 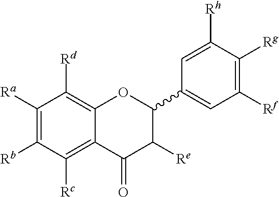 | OH | H | H | H | OH | H, OH | OH | OH |
| 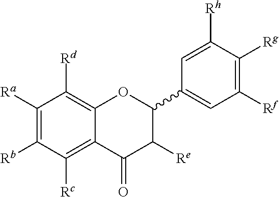 | OH | H | OH | H | H | H | H, OH, —O—CH₃ | H |
| 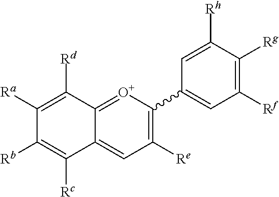 | OH | H | OH | H | OH | —O—CH₃ | OH | —O—CH₃ |
| 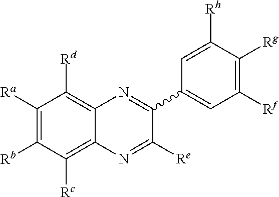 | H, CH₃ | H. CH₃ | H | H | H | H | OH | OH |
| 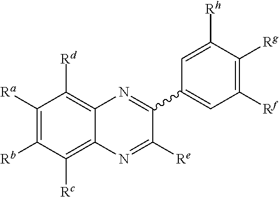 | H, CH₃ | H. CH₃ | H | H | H | OH | OH | H |
| 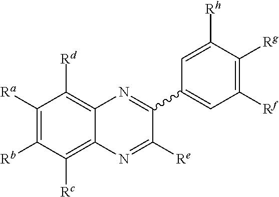 | H | H | H | H | H | H | O—C(═O)—CH₃ | H |

Further, the following compounds are preferred: 2-Hydroxy-5-(6,7,8-trimethyl-quinoxalin-2-yl)-benzoic acid, 3-(6,7-Dimethyl-quinoxalin-2-yl)-6-hydroxy-2-methyl-benzoic acid, 6-Hydroxy-2-methyl-3-(6,7,8-trimethyl-quinoxalin-2-yl)-benzoic acid, 5-(6,7-Dimethyl-4-trifluoromethyl-quinolin-3-yl)-2-hydroxy-benzoic acid, 4-(4-Amino-2-methyl-pteridin-6-yl)-benzene-1,2-diol, 4-(1,3-Dioxa-5,8-diaza-cyclopenta[b]naphthalen-6-yl)-benzene-1,2-diol, 2-Amino-5-(6,7-dimethyl-quinoxalin-2-yl)-3-hydroxy-benzoic acid, 3-(6,7-Dimethyl-quinoxalin-2-yl)-5-hydroxy-benzoic acid, 2-(3,4-Dihydroxy-phenyl)-quinoxaline-6,7-diol, 2-(3,4-Dihydroxyphenyl)-6,7-dimethylquinoxaline, 5-(6,7-Dimethyl-quinoxalin-2-yl)-benzene-1,3-diol, 2-(6,7-Dimethyl-quinoxalin-2-yl)-benzene-1,4-diol, 5-(6,7-Dimethyl-quinoxalin-2-yl)-2-hydroxy-benzoic acid, 4-(6,7-dimethylquinoxalin-2-yl)benzene-1,2-diol, 4-(7-Hydroxy-6-methyl-quinoxalin-2-yl)-benzene-1,2-diol, 2-(3,4-Dihydroxy-phenyl)-quinoxaline-5,7-diol, -(3,4-dihydroxyphenyl)-3,7-dihydroxy-2,3-dihydro chromen-4-one (Fustin), 3,7-Dihydroxy-2-(3,4,5-trihydroxy-phenyl)-chroman-4-one, 3,5,7-Trihydroxy-2-(3,4,5-trihydroxy-phenyl)-chroman-4-one, and 4-(quinoxalin-2-yl)phenyl acetate

According to an alternative embodiment, the compound is selected from the group consisting of:

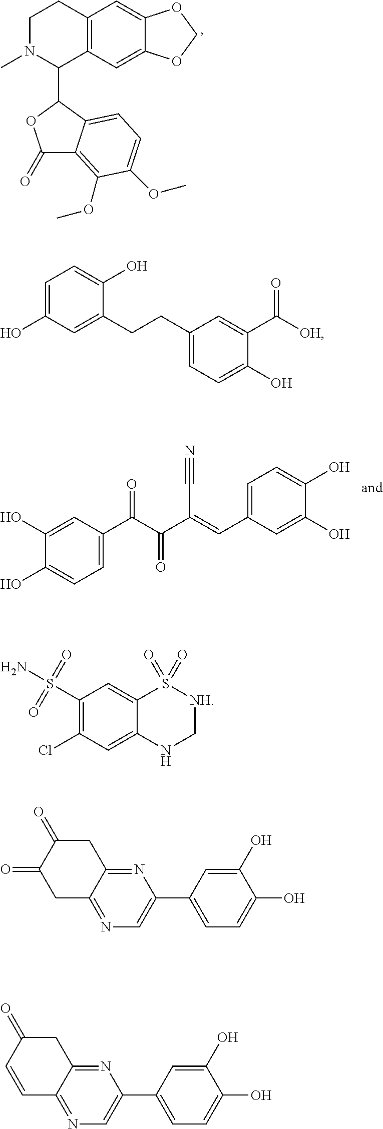 [SMILES: COc1ccc2c(c1OC)C(=O)OC2C1c2cc3c(cc2CCN1C)OCO3 ; O=C(O)c1cc(CCc2cc(O)ccc2O)ccc1O ; N#C/C(=C\c1ccc(O)c(O)c1)C(=O)C(=O)c1ccc(O)c(O)c1 ; NS(=O)(=O)c1cc2c(cc1Cl)NCNS2(=O)=O ; O=C1Cc2ncc(-c3ccc(O)c(O)c3)nc2CC1=O ; O=C1C=Cc2ncc(-c3ccc(O)c(O)c3)nc2C1]

### Pharmaceutically Acceptable Salt

As described above, the compounds of the present invention can be formulated as pharmaceutically acceptable salt or solvate.

Typical pharmaceutically acceptable salts include those salts prepared by reaction of the compounds of the present invention with a pharmaceutically acceptable mineral or organic acid or an organic or inorganic base. Such salts are known as acid addition and base addition salts. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, napththalene-2-sulfonate, mandelate and the like. Preferred pharmaceutically acceptable acid addition salts are those formed with mineral acids such as hydrochloric acid and hydrobromic acid, and those formed with organic acids such as maleic acid and methanesulfonic acid. Salts of amine groups may also comprise quaternary ammonium salts in which the amino nitrogen carries a suitable organic group such as an alkyl, alkenyl, alkynyl, or aralkyl moiety. Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt foul's are particularly preferred. It should be recognized that the particular counter ion forming a part of any salt of this invention is usually not of a critical nature, so long as the salt as a whole is pharmacologically acceptable and as long as the counter ion does not contribute undesired qualities to the salt as a whole.

The term acceptable solvate encompasses also pharmaceutically acceptable solvates of the compounds of the invention, wherein the compound combines with a solvent such as water, methanol, ethanol or acetonitrile to form a pharmaceutically acceptable solvate such as the corresponding hydrate, methanolate, ethanolate or acetonitrilate.

Moreover, the present invention relates to a method for identifying a C4 plant selective herbicide, comprising screening a compound library for a compound, which is
i) capable of binding to the malate binding site comprised by a phosphoenolpyruvate carboxylase from a C4 plant, thereby inhibiting the activity of said phosphoenolpyruvate carboxylase, and
ii) not capable of binding to the malate binding site comprised by a phosphoenolpyruvate carboxylase from a C3 plant.

A compound which is capable of binding to the malate binding site comprised by the phosphoenolpyruvate carboxylase from a C4 plant, but which is not capable of binding to the malate binding site comprised by the phosphoenolpyruvate carboxylase from a C3 plant, preferably, is a candidate compound that can be used as a C4 selective herbicide.

The term “herbicide” is well understood in the art. As used herein, the term refers to a compound that inhibits the growth of plants. The inhibition of the growth of plants shall include all deviations from natural development. In particular, the term refers to reducing the growth of the plant or to kill a plant. Moreover, the term may include inhibition of the development. Preferably, the term further includes dwarfing.

The herbicide to be identified by the method of the present invention shall be a C4 plant selective herbicide. A C4 plant selective herbicide, preferably, shall inhibit the growth of a C4 plant, but shall not inhibit the growth of a C3 plant. Also preferably, a C4 plant selective herbicide shall significantly inhibit the growth of a C4 plant, whereas growth of C3 plant shall remain unchanged, in particular essentially unchanged, in the presence of said herbicide (as compared to the absence of the herbicide). Whether growth of a plant is essentially unchanged or significantly inhibited can be determined by the skilled person without further ado.

As set forth above, the term “inhibiting the growth” also includes a “reducing the growth”. Accordingly, it is envisaged that the growth of a C4 shall be reduced in the presence of said herbicide (in particular in the presence of an effective amount) as compared to the growth in the in the absence of said herbicide. In contrast, the growth of a C3 plant shall not be affected by the presence of the herbicide, in particular, shall not be significantly affected in the presence of said herbicide. Accordingly, a C3 plant, preferably, shows the same growth (in particular, essentially the same growth), regardless whether the herbicide is present or not. Thus, it is envisaged, that a C4 plant selective herbicide shall reduce the growth of a C4 plant, but shall not reduce the growth of a C3 plants.

It is to be understood that the inhibition of the growth of the C4 plant may dependent on the concentration. Preferably, the growth of the C4 plant is inhibited if the plant is contacted with an effective amount of the herbicide, i.e. with a herbicidal effective amount. Whether an amount of the herbicide is effective, or not, can be determined by methods known in the art.

The terms “C3 plant” and “C4 plant” are well understood by the skilled person.

A C3 plant, in particular, shall be a plant in which the CO₂ is first fixed into a compound containing three carbon atoms (phosphoglyceric acid) before entering the Calvin cycle of photosynthesis. Preferably, it uses the C3 carbon fixation pathway as the sole mechanics to covert CO₂ into 3-phosphoglycerate. C3 plants are well known in the art and include tobacco, tomato, soybeans, potato, cucumber, cotton, wheat, rice and barley.

Particularly preferred C3 plants are Oryza sativa, Hordeum vulgare, Brassica napus, Triticum aestivum, and, in particular, Flaveria pringlei. Further preferred are Glycine max (soybean), cucumber, grapes, potato, tomato, cassava, sugar beet and watermelon. Moreover, it is also preferred that the C3 plant is selected from the group of C3 plants consisting of asparagus, green beans, cabbages and other brassicas, carrots and turnips, cassava, cauliflowers and broccoli, green chilies and peppers, cucumbers and gherkins, eggplants, grapes, lettuce and chicory, pumpkins, squash and gourds, spinach, sugar beet, sunflower seed, watermelons, and yams.

A C4 is plant in which the CO₂ is first fixed into a compound containing four carbon atoms before entering the Calvin cycle of photosynthesis. In particular, a C4 plant shall utilize the C4 carbon fixation pathway in which the CO₂ is first bound to a phosphoenolpyruvate resulting in the formation of four-carbon compound (oxaloacetate). C4 plants are well known in the art and include monocotyledonous plants such as maize, sugarcane, and sorghum, as well as dicotyledonous plants such as Amaranthus.

Preferably, the C4 plants belong to the C4 plants of the family selected from Aizoaceae (Genus, in particular, Cypselea, Gisekia, Trianthema, Zalaeya), Amaranthaceae (Acanthochiton, Aerva, Alteranthera, Amaranthus, Brayulinea), Caryophyllaceae (Polycarpaea), Chenopodiacea (Anabis, Aellenia, Arthrophytum, Atriplex, Bassia, Bienerta, Camphorosma, Chenolea, Climacoptera, Comulaca, Cytobasis, Echinopsilon, Garnanthus, Girgensohnia, Halanthium, Halimocnemis, Halocharis, Halogeton, Halostigmaria, Haloxylon, Hammada, Horaninovia, Hypocyclix, Kochia, Londesia, Noaea, Panderia, Petrosimonia, Salsola, Seidltzia, Suaeda, Theleophyton, Traganum) Molluginaceae (Glinis, Mollugo) Nyctaginaceae (Ilionia, Boerhaavia, Okenia), Portulaceae (Portulaca), Polygonaceae (Calligonum), Euphorbiaceae (Chamaesyce, Euphorbia) Capparaceae (Gynandropsis), Zygophyllaceae (Kallstroemia, Tribulus, Zygophyllum) Asteraceae (Glossocordia, Glossogyne, Isostigma, Pectis), Boraginaceae, Convolvulaceae, Acanthaceae, Scrophulariaceae, Poaceae (Alloteropsis, Andropogon, Arundinella, Bouteloua, Cynodon, Echinochloa, Leptochloa, Microstegium, Panicum, Paspalum, Setaria, Sorghum, Spartina, Sporobolus, Zea). Preferred genera are indicated in brackets.

Particularly preferred C4 plants are selected from the group consisting of Bothriochloa saccharoides, Bothriochloa ischaemum, Imperata cylindrica (Cogon grass), Panicum capillare (Witchgrass), Panicum coloratura, Panicum fluviicola, Panicum miliaceum, Panicum phragmitoides, Panicum turgidum, Saccharum officinarum (Wild sugarcane), Sorghum bicolor, Zea mays (Maize), in particular Flaveria trinervia, Setaria italica (Foxtail millet), Setaria palmifolia, Setaria plicata, Paspalum conjugatum (Buffalo Gras), Paspalum quadrifarium, Paspalum dilatatum, Amaranthus hypochondriacus, Amaranthus spinosus (spiny amaranth), Sorghum halepense (Johnsongrass), Rottboellia cochinchinensis (itchgrass), Commelina benghalensis (tropical spiderwort), Trianthema portulacastrum (desert horse purslane), Ageratum conyzoides (Chick weed, Goatweed, Whiteweed), Bidens pilosa (Spanish Needle), Euphorbia hirta, Portulaca oleracea (Pigweed). Further preferred C4 plants are Alopecurues myosuroides and Gallium aparine. Particularly preferred C4 plants are Flaveria trinervia, Flaveria australasica, Zea mays, and Sorghum bicolor.

The phosphoenolpyruvate carboxylases to be used in the context of the method of the present invention shall be derived from a C3 and from a C4 plant, respectively. Thus, it is particular envisaged that the phosphoenolpyruvate carboxylase to be used is naturally present in a C3 and a C4 plant, respectively. Preferred C3 and C4 plants are disclosed herein above.

The term “phosphoenolpyruvate carboxylase” is well understood by the skilled person. Frequently, phosphoenolpyruvate carboxylases are also referred to as PEP carboxylase, PEPCase, or PEPC. A phosphoenolpyruvate carboxylase as referred to herein shall be capable of catalyzing the formation of oxaloacetate from phosphoenolpyruvate (PEP) and bicarbonate (EC 4.1.1.31). In particular, a phosphoenolpyruvate carboxylase shall be capable of catalyzing the irreversible beta-carboxylation of phosphoenolpyruvate by bicarbonate to yield oxalacetate and phosphate. The aforementioned enzymatic activity can be determined by assays well known in the art.

It is well known in the art, that the regulation of the PEP carboxylase is, inter alia, effected by allosteric effectors, L-aspartate and L-malate. L-aspartate or L-malate bind at the enzyme to an allosteric binding domain, the malate binding site (also called “aspartate binding site” or “aspartate/malate binding site”), thereby inhibiting the activity of the PEP carboxylase. The malate binding site of a PEP carboxylase comprises four conserved residues which participate directly in the binding of malate and aspartate. The conserved residues are well known in the art, and e.g. described by Jacobs et al. (Plant Cell and Environment (2008), 31, 793-803). In Flaveria, the four conserved residue which participate in the binding of aspartate/malate are R641, K829, R888 and N964 (see the sequences disclosed herein for the PEPC from Flaveria pringlei and trinervia.

In a preferred embodiment of the present invention the PEP carboxylase from a C4 plant is derived from Flaveria trinervia (GenBank Accession Number CAA43601.1 GI:498699), and the PEP carboxylase from a C3 plant is derived from Flaveria pringlei (GenBank Accession number CAA45505.1 G1:18458), i.e. two highly homologous PEP carboxylases.

The amino acid sequence of the PEP carboxylase from Flaveria trinervia is shown in SEQ ID NO: 1, the amino acid sequence of the PEP carboxylase from Flaveria pringlei is shown in SEQ ID NO: 2.

Thus, in a preferred embodiment, the phosphoenolpyruvate carboxylase derived from a C4 plant is, in particular, encoded by a polynucleotide comprising a nucleic acid selected from the group consisting of:
a. a nucleic acid encoding a polypeptide having an amino acid sequence as shown in SEQ ID No: 1;
b. a nucleic acid encoding a polypeptide having an amino acid sequence being at least 90%, 95%, 97% or in particular 99% identical to the amino acid sequence shown in SEQ ID No: 1, wherein said polypeptide has phosphoenolpyruvate carboxylase activity.

The enzymatic activity of a PEP carboxylase is disclosed elsewhere herein. Thus, the phosphoenolpyruvate carboxylase shall be capable of catalyzing the formation of oxaloacetate from phosphoenolpyruvate (PEP) and bicarbonate (EC 4.1.1.31, see above). Preferably, the phosphoenolpyruvate carboxylase derived from a C4, comprises, within its malate binding site, a glycine residue which corresponds to the conserved glycine residue found at position 884 of the PEP carboxylase from Flaveria pringlei. Moreover, the inventors have found that some of the PEP carboxylases from C4 plants comprise at a position which corresponds to the glycine residue, a serine, glutamine or isoleucine residue, i.e. amino acid residues which will also not inhibit binding of the identified herbicides by their side-chain. Alternatively, the said phosphoenolpyruvate carboxylase derived from a C4, comprises, within its malate binding site, a serine, glutamine or isoleucine residue which corresponds to said glycine residue. The malate binding site of the PEP Carboxylase from Flaveria trinervia is well known in the art, and, e.g. described by Jacobs et al. (Plant Cell and Environment (2008), 31, 793-803). Preferably, the variants of the phosphoenolpyruvate carboxylase set forth in c. and d. above are capable of binding malate.

Thus, in a preferred embodiment, the phosphoenolpyruvate carboxylase derived from a C3 plant is, in particular, encoded by a polynucleotide comprising a nucleic acid selected from the group consisting of:
a. a nucleic acid encoding a polypeptide having an amino acid sequence as shown in SEQ ID No: 2;
b. a nucleic acid encoding a polypeptide having an amino acid sequence being at least 90%, 95%, 97% or in particular 99% identical to the amino acid sequence shown in SEQ ID No: 2, wherein said polypeptide has phosphoenolpyruvate carboxylase activity.

The activity of a PEP carboxylase is disclosed elsewhere herein. Preferably, the phosphoenolpyruvate carboxylase derived from a C3, comprises, within its malate binding site, a conserved arginine residue which corresponds to the conserved arginine residue found at position 884 of the PEP carboxylase from Flaveria pringlei. Preferably, the conserved arginine residue is within the region of amino acid 875 to 895. The malate binding site of the PEP Carboxylase from Flaveria pringlei is well known in the art, and, e.g. described by Jacobs et al. (Plant Cell and Environment (2008), 31, 793-803), which is herewith incorporated by reference with respect to its entire disclosure content. Preferably, the variants of the phosphoenolpyruvate carboxylase set forth in b. above are capable of binding malate.

The term “polynucleotide” as used herein refers to a linear or circular nucleic acid molecule. It encompasses DNA as well as RNA molecules. The polynucleotide of the present invention shall be provided, preferably, either as an isolated polynucleotide (i.e. isolated from its natural context) or in genetically modified form. The term encompasses single as well as double stranded polynucleotides. Moreover, comprised are also chemically modified polynucleotides including naturally occurring modified polynucleotides such as glycosylated or methylated polynucleotides or artificial modified one such as biotinylated polynucleotides. The polynucleotide of the present invention is characterized in that it shall encode a polypeptide as referred to above. The polynucleotide, preferably, has a specific nucleotide sequence as mentioned above. Moreover, due to the degeneracy of the genetic code, polynucleotides are encompassed which encode a specific amino acid sequence as recited above.

Moreover, the term “polynucleotide” as used in accordance with the present invention further encompasses variants of the aforementioned specific polynucleotides. Said variants may represent orthologs, paralogs or other homologs of the polynucleotide of the present invention. The polynucleotide variants, preferably, comprise a nucleic acid sequence characterized in that the sequence can be derived from the aforementioned specific nucleic acid sequences by at least one nucleotide substitution, addition and/or deletion whereby the variant nucleic acid sequence shall still encode a polypeptide having the activity as specified above. Variants also encompass polynucleotides comprising a nucleic acid sequence which is capable of hybridizing to the aforementioned specific nucleic acid sequences, preferably, under stringent hybridization conditions. These stringent conditions are known to the skilled worker and can be found in Current Protocols in Molecular Biology, John Wiley & Sons, N. Y. (1989), 6.3.1-6.3.6. A preferred example for stringent hybridization conditions are hybridization conditions in 6× sodium chloride/sodium citrate (=SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of the polypeptides of the present invention. Conserved domains of the polypeptide of the present invention may be identified by a sequence comparison of the nucleic acid sequence of the polynucleotide or the amino acid sequence of the polypeptide of the present invention with sequences of other members of the enzyme families referred to in accordance with this invention. Oligonucleotides suitable as PCR primers as well as suitable PCR conditions are described in the accompanying Examples. As a template, DNA or cDNA from bacteria, fungi, plants or animals may be used. Further, variants include polynucleotides comprising nucleic acid sequences which are, in increasing order of preference, at least 70%, at least 75%, at least 80%, at least 85%, at least 90%, at least 95%, at least 98% or at least 99% identical to the specific nucleic acid sequences. Moreover, also encompassed are polynucleotides which comprise nucleic acid sequences encoding amino acid sequences which are, in increasing order of preference, at least 70%, at least 75%, at least 80%, at least 85%, at least 90%, at least 95%, at least 98% or at least 99% identical to the specific amino acid sequences referred to herein. The percent identity values as set forth herein are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS, 5 1989: 151-153) or the programs Gap and BestFit (Needleman 1970, J. Mol. Biol. 48; 443-453 and Smith 198, Adv. Appl. Math. 2; 482-489), which are part of the GCG software packet from Genetics Computer Group, 575 Science Drive, Madison, Wis., USA 53711, version 1991, are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the aforementioned nucleic acid sequences is also encompassed as a polynucleotide of the present invention. The fragment shall encode a polypeptide which still has the activity as specified above. Accordingly, the polypeptide may comprise or consist of the domains of the polypeptide of the present invention conferring the said biological activity. A fragment as meant herein, preferably, comprises at least 50, at least 100, at least 250 or at least 500 consecutive nucleotides of any one of the aforementioned nucleic acid sequences or encodes an amino acid sequence comprising at least 20, at least 30, at least 50, at least 80, at least 100 or at least 150 consecutive amino acids of any one of the aforementioned amino acid sequences.

The polynucleotides of the present invention either essentially consist of the aforementioned nucleic acid sequences or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Such fusion proteins may comprise as additional part peptide sequences for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and comprise FLAG-tags, 6-histidine-tags, MYC-tags and the like.

Further preferred PEP carboxylases from C3 plants are derived from the following plants (indicated is the organism, the GenBank Accession Number, and the position of the conserved arginine residue. For some enzymes, only the sequences of fragments are known, the full length sequences can be determined by the skilled person without further ado):

| | |
|---|---|
| Oryza sativa (Rice) EMBL AAG00180.1 | R879 |
| Brassica napus (Rape) TrEMBL Q42634 | R883 |
| Triticum aestivum (Wheat) EMBL CAA07610.1 | R891 |
| Phaseolus vulgaris (Kidney bean) Q9AU12 | R887 |
| Hordeum vulgare (Barley) EMBL ABB01326.1 (Fragment) | R146 |
| Vicia faba (Broad bean) (Faba vulgaris) EMBL CAA09588.1 | R885 |
| Cucumis sativus (Cucumber) EMBL CAD10147.1 (Fragment) | R117 |
| Vitis vinifera (Grape) EMBL AAL83719.1 (Fragment) | R258 |
| Solanum tuberosum (Potato) P29196 | R885 |
| Gossypium hirsutum (Upland cotton) EMBL AAB80714.1 | R884 |
| Glycine max (Soybean) P51061 [UniParc] | R886 |
| Nicotiana tabacum (Common tobacco) P27154 [UniParc] | R884 |

Further preferred PEP carboxylases from C3 plants are derived from Hordeum vulgare (BAJ88050.1 GI:326516054) or from Arabidopsis thaliana (AAC24594.1 GI:3264805)

Further preferred PEP carboxylases from C4 plants are derived from the following plants (indicated is the organism, the GenBank Accession Number, and the position of the conserved glycine residue, alternatively of the senile, glutamine or isoleucine, see elsewhere herein. For some enzymes, only the sequences of fragments are known, the full length sequences can be determined by the skilled person without further ado):

| | |
|---|---|
| Bothriochloa saccharoides (Fragment) TrEMBL A7DX44 | G429 |
| Bothriochloa ischaemum (Fragment) TrEMBL A7DX82 | G429 |
| Imperata cylindrica (Cogon grass) Fragment) TrEMBL A7DX63 | G429 |
| Panicum capillare (Witchgrass) (Fragment) EMBL CAM84110.1 | G429 |
| Panicum coloratum (Fragment) TrEMBL A7DXB1 | G429 |
| Panicum fluviicola TrEMBL (Fragment) D9UAH3 | G721 |
| Panicum miliaceum (Fragment) TrEMBL E1XUD1 | G721 |
| Panicum phragmitoides (Fragment) TrEMBL D9UAH8 | G721 |
| Panicum turgidum (Fragment) TrEMBL D9UAH5 | G721 |
| Saccharum officinarum (Wild sugarcane) EMBL CAC85930.1 | G881 |
| Sorghum bicolor Swiss-prot P15804 | G881 |
| Zea mays (Maize) EMBL CAA33317.1 | G890 |
| Flaveria trinervia Swiss-Prot P30694 | G884 |
| Panicum laetum (Fragment) EMBL CAM84117.1 | S429 |
| Setaria italica (Foxtail millet) TrEMBL Q8S2Z8 | Q884 |
| Setaria palmifolia (Fragment) TrEMBL A7DXC5 | Q429 |
| Setaria plicata (Fragment) TrEMBL A7DXC6 | Q429 |
| Paspalum conjugatum (Buffalo Gras) (Fragment) TrEMBL | Q429 |
| A7DXB6 | |
| Paspalum quadrifarium (Fragment) EMBL CAM84120.1 | Q429 |
| Paspalum dilatatum (Fragment) TrEMBL A7DXB7 | Q429 |
| Amaranthus hypochondriacus SwissProt Q43299 | I884 |

In the context of the present invention, a compound library shall be screened. The term “compound library” as used herein refers to any collection of compounds that includes a plurality of molecular structures. Compound libraries preferably, included combinatorial chemical libraries or natural products libraries. Preferably, the library is a small compound library. Most preferably, the library is selected from the following a library selected from the Structure-Data File (SDF) from ChemBank (Strausberg, R. L.; Schreiber, S. L. Science 2003, 300(5617), 294-295), the Ligand database of the Kyoto Encyclopedia of Genes and Genomes (Goto S.; Okuno Y.; Hattori M.; Nishioka T.; Kanehisa M. Nucleic Acids Res. 2002, 30(1), 402-404), and the Open NCI database (Voigt, J. H.; Bienfait, B.; Wang, S.; Nicklaus, M. C. J. Chem. Inf. Comput. Sci. 2001, 41(3), 702-712).

In a preferred embodiment of the method of the present invention, the screening the compound library comprises the steps of
(a) providing the three-dimensional structure of the PEP carboxylase or of the malate binding site of a phosphoenolpyruvate carboxylase from a C4 plant,
(b) providing the three-dimensional structure of the PEP carboxylase or of the malate binding site phosphoenolpyruvate carboxylase from a C3 plant,
(c) providing a three-dimensional structure of the compound to be tested, and
(d) identifying, based on the three-dimensional structures provided in steps (a), (b), and/or (c), a compound which is capable of binding to the malate binding site comprised by the phosphoenolpyruvate carboxylase from a C4 plant, but which is not capable of binding to the malate binding site comprised by the phosphoenolpyruvate carboxylase from a C3 plant.

The three-dimensional structure of the PEP carboxylase to be provided, preferably, is the tertiary structure of said PEP carboxylase or of the malate binding site comprised by said PEP carboxylase. How to determine the tertiary structure of a polypeptide is well known in the art. Moreover, how to determine the structure of a compound is general is well known in the art, e.g. by nuclear magnetic resonance (NMR) spectroscopy. Thus, the three-dimensional structure of the compound to be tested and/or of the PEP carboxylase or of the malate binding site of the phosphoenolpyruvate carboxylase is determined (and, thus, provided) experimentally. It is, particularly, envisaged to determine the three-dimensional structure of the PEP carboxylase or of the malate binding site of the phosphoenolpyruvate carboxylase experimentally. Therefore, the step of “providing a three-dimensional structure” may be anteceded by a step of determining the three-dimensional structure experimentally (in particular in steps a) and b) as set forth above, but also in step c).

Preferred techniques for determination the tertiary structure of PEP carboxylase or of the malate binding site comprised by said PEP carboxylase are protein crystallography, NMR spectroscopy, electron microscopy (in particular Cryo-EM) and the rapidly developing method of small-angle X-ray scattering (SAXS). These techniques are well known in the art and, e.g., described by Lottspeich F and Zorbas H (eds.) Bioanalytik Spektrum Akademischer Verlag: Heidelberg, 1998, and Tsuruta H and Johnson, J J E (2001). “Small-angle X-ray scattering” in International Tables for Crystallography, Volume F: Macromolecular Crystallography (eds, M. G. Rossmann, E. Arnold), Kluwer Academic Publisher, Chapter 19.3, pp 428-437.

The identification, whether a compound to be tested is capable of binding to the malate binding site, or not, is preferably, based on the three-dimensional structures provided in steps (a) and (c), and (b) and (c), respectively. Preferably, the identification is done by applying the key-lock principle. In particular, a compound that is tested which fits into the malate binding site of a PEP carboxylase shall be capable of binding to said binding site, whereas a compound that does not fit into the malate binding site of a PEP carboxylase shall not be capable of binding to said binding site. This assessment is, preferably, carried out in silico. How to carry out this assessment is well known in the art and, e.g., described by Yuriev E, Agostino M and Ramsland P A (2011) Challenges and advances in computational docking: 2009 in review. J Mol Recognit. 24(2):149-64. doi: 10.1002/jmr.1077, and by Trott, O. and Olson, A. J. AutoDock Vina: improving the speed and accuracy of docking with a new scoring function, efficient optimization and multithreading, Journal of Computational Chemistry 31 (2010) 455-461 all of which are hereby incorporated by reference with respect their entire disclosure content.

A compound which is capable of binding to the malate binding site comprised by the phosphoenolpyruvate carboxylase from a C4 plant, but which is not capable of binding to the malate binding site comprised by the phosphoenolpyruvate carboxylase from a C3 plant, preferably, is a compound that can be used as a C4 selective herbicide.

Preferably, the compound which is capable of binding to the malate binding site comprised by the phosphoenolpyruvate carboxylase from a C4 plant, but which is not capable of binding to the malate binding site comprised by the phosphoenolpyruvate carboxylase from a C3 plant does not bind to the malate binding site comprised by a phosphoenolpyruvate carboxylase from a C3 plant due to steric and/or electrostatic interactions with the conserved arginine residue comprised by said malate binding site. It is particularly envisaged that the said compound does not bind to the malate binding site due to steric interaction with the conserved arginine residue comprised by said malate binding site. Accordingly, binding of said compound is, preferably, inhibited by the presence of said conserved arginine residue.

Alternatively, or additionally screening of the compound library, preferably, comprises the steps of
(u) contacting a compound comprised by the compound library to be tested with a phosphoenolpyruvate carboxylase from a C3 plant, and assessing whether said compound binds to said malate binding site of the phosphoenolpyruvate carboxylase from said C3 plant, and
(v) contacting said compound with a phosphoenolpyruvate carboxylase from a C4 plant, and assessing whether said compound binds to said malate binding site of the phosphoenolpyruvate carboxylase from said C4 plant.

In particular, the screening of the compound library, preferably, comprises the steps of
(u) contacting a compound identified by carrying out steps (a), (b), (c) and (d) as set forth above the with a phosphoenolpyruvate carboxylase from a C3 plant, and assessing whether said compound binds to said malate binding site of the phosphoenolpyruvate carboxylase from said C3 plant, and
(v) contacting said compound with a phosphoenolpyruvate carboxylase from a

C4 plant, and assessing whether said compound binds to said malate binding site of the phosphoenolpyruvate carboxylase from said C4 plant.

Thereby, it can be verified whether the identified compound is capable of binding to the malate binding site comprised by the phosphoenolpyruvate carboxylase from a C4 plant, and whether said compound is not capable of binding to the malate binding site comprised by the phosphoenolpyruvate carboxylase from a C3 plant.

The assessment whether a compound binds to a malate binding site can be carried out in vivo or in vitro. Methods for carrying out this assessment are well known in the art, and include Isothermal Titration calorimetry (ITC) for measuring biomolecular interactions as disclosed by Chaires J. B. (2008) calorimetry and thermodynamics in drug design. Arrau Rev Biophys 37, 135-151. Further preferred methods are disclosed by Cooper M. A. (2003) Label-free screening of bio-molecular interactions. Anal Bioanal Chem 377, 834-842.

In a preferred embodiment of the present invention, the assessment whether said compound binds to said malate binding site of phosphoenolpyruvate carboxylase from said C3 or C4 plant is done by determining the enzymatic activity of said (C3 or C4) phosphoenolpyruvate carboxylase after contacting the compound with the phosphoenolpyruvate carboxylase. Preferably, a compound binds to the malate binding site of said PEP carboxylase (in particular of a C4 PEP carboxylase) if the activity of said PEP carboxylase is reduced i) after contacting said compound with said PEP carboxylase and/or ii) in comparison of a PEP carboxylase that has not been contacted with the compound. Preferably, a compound does not bind to the malate binding site of said PEP carboxylase (in particular of a C3 PEP carboxylase) if the activity of said PEP carboxylase is unchanged, in particular, essentially unchanged i) after contacting said compound with said PEP carboxylase and/or ii) in comparison of a PEP carboxylase that has not been contacted with the compound.

How to determine the activity of a PEP carboxylase is well known in the art. E.g, the formation of oxaloacetate can be determined spectrophotometrically, e.g., in a malate dehydrogenase coupled system. The reaction velocity can be measured as a decrease in A₃₄₀ resulting from the oxidation of NADH.

Finally, the method of the present invention, preferably, may further comprise the steps of
x) contacting a C3 plant with a compound identified by the steps as described herein above, in particular, with a compound which is capable of binding to the malate binding site comprised by the phosphoenolpyruvate carboxylase from a C4 plant, but which is not capable of binding to the malate binding site comprised by the phosphoenolpyruvate carboxylase from a C3 plant,
y) contacting a C4 plant with said compound, and
z) assessing whether the compound inhibits the growth of said C3 and/or of said C4 plant.

Thereby, it can be verified, whether an identified compound can be used as C4 selective herbicide. Preferably, a compound which inhibits the growth of a C4 plant, but which does not inhibit the growth of the C3 plant can be used as C4 selective herbicide.

The figures show:

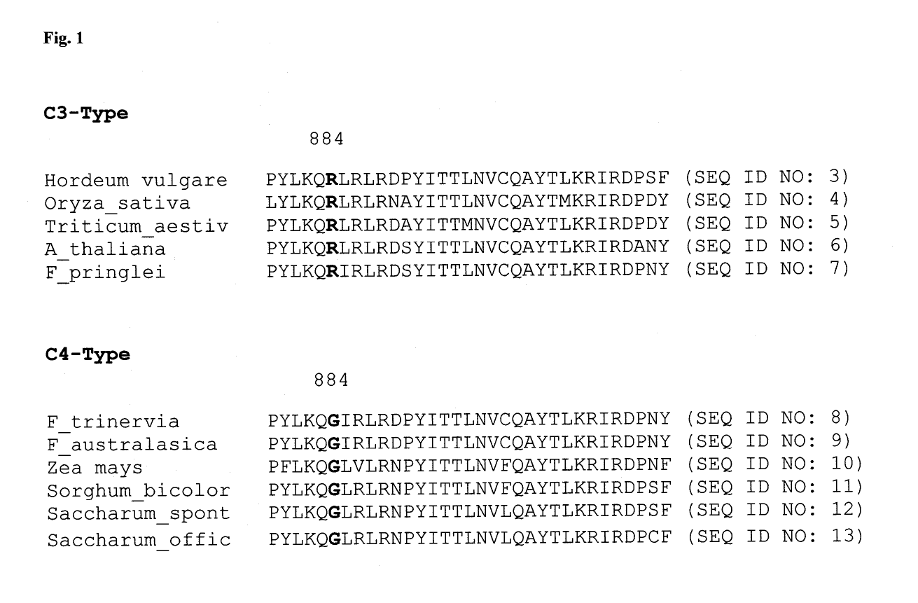

FIG. 1: Partial alignment of PEP carboxlyases derived from various C3 and C4 plants. Indicated are the conserved arginine residues in C3 PEP carboxylases, and the conserved glycine residues in C4 PEP carboxylases.

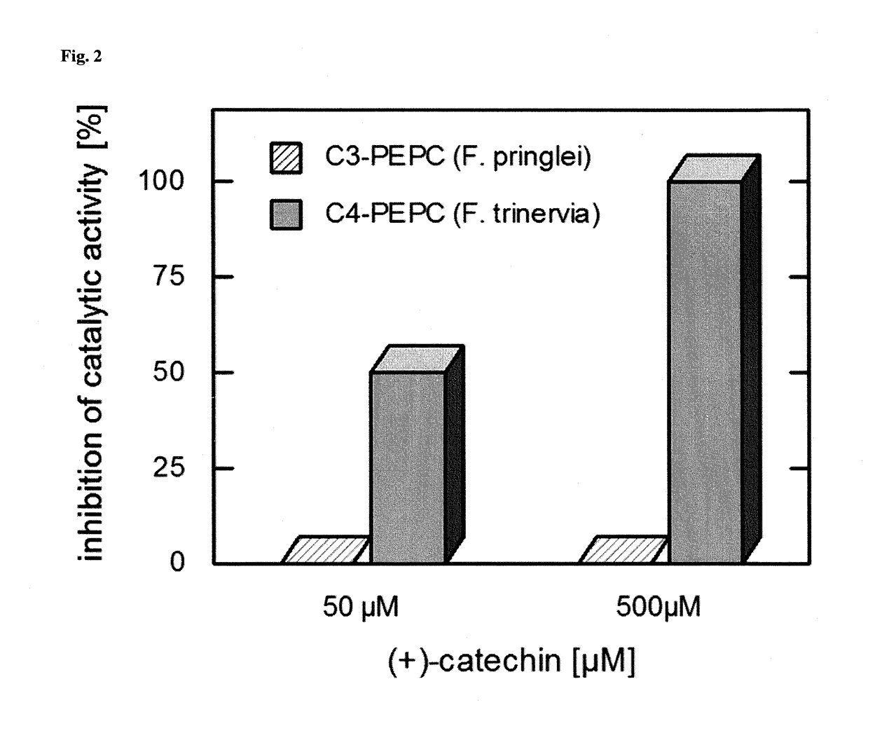

FIG. 2: Selective Inhibition of a C4-PEPC by (+)-catechin (measured at a concentration of 50 micromolar and 500 micromolar. In table 2 the IC50 value determined from inhibition studies at different concentrations of (+)-catechin is given. Experiments were done in triplicate with at least seven different concentrations of the inhibitor.)

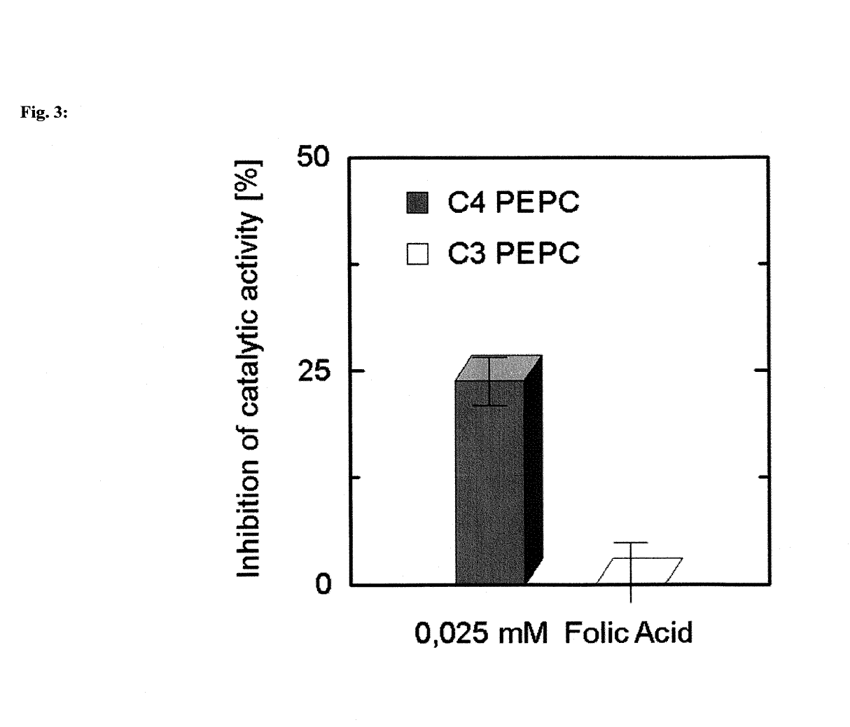

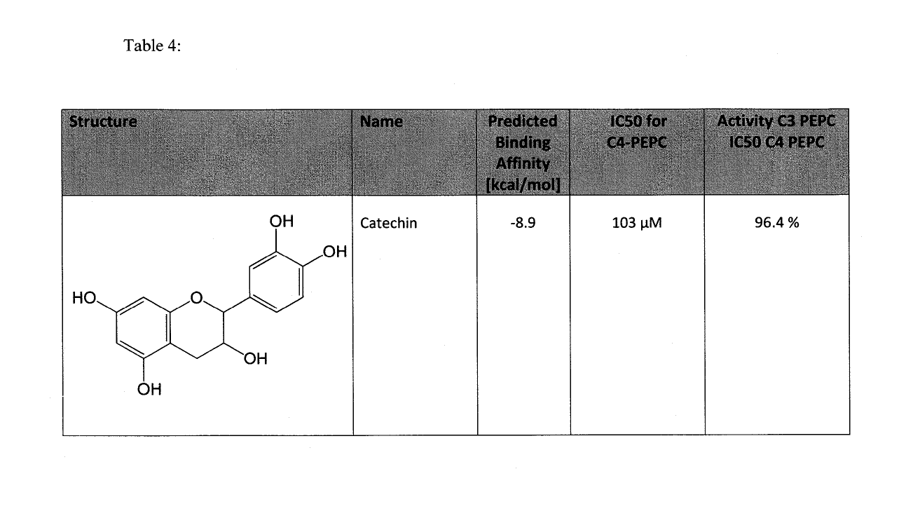

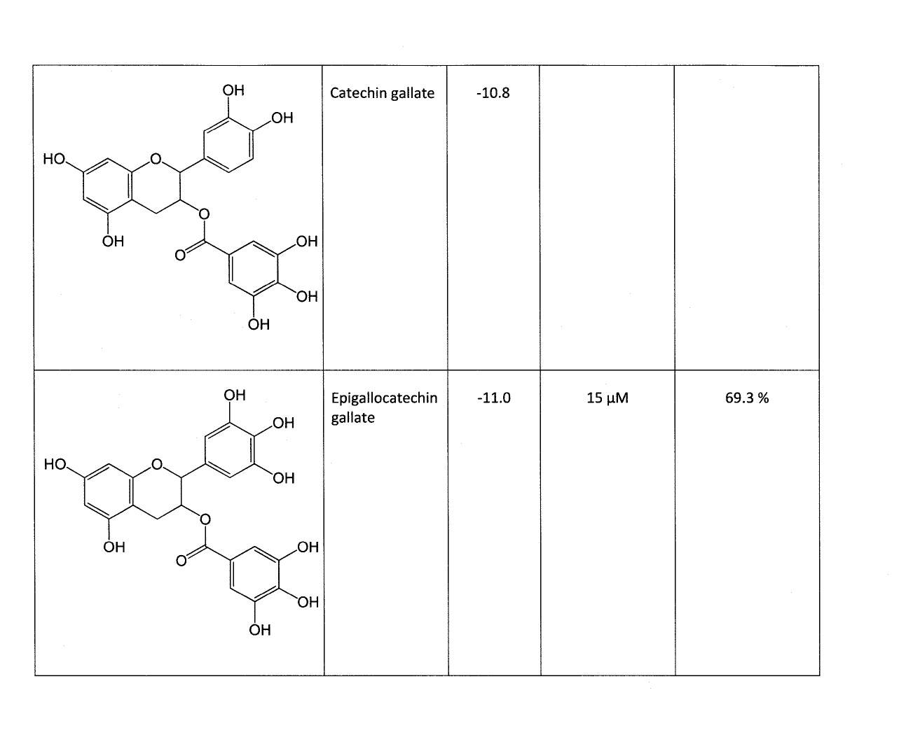

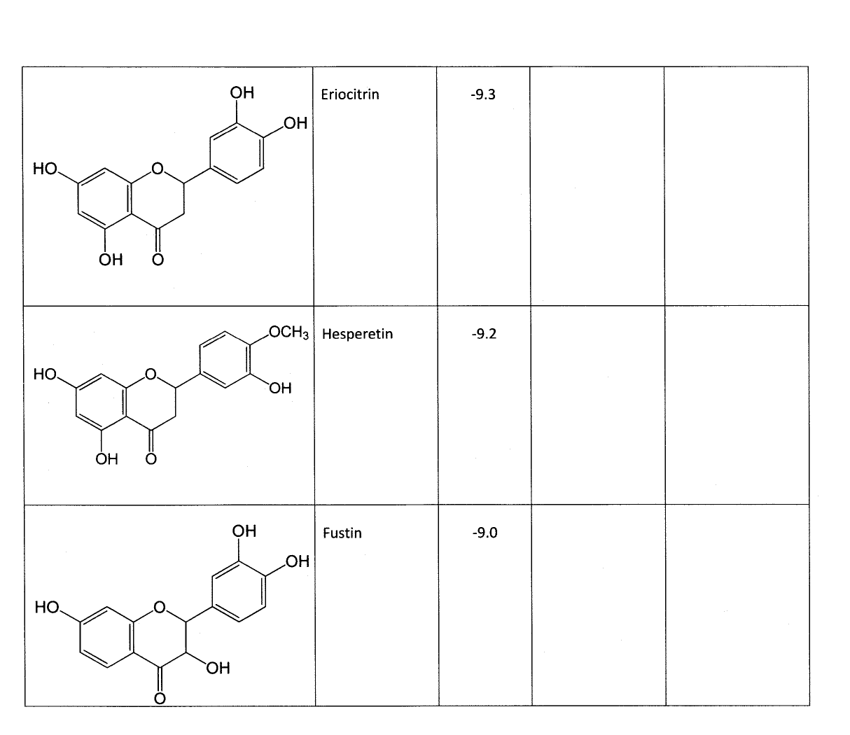

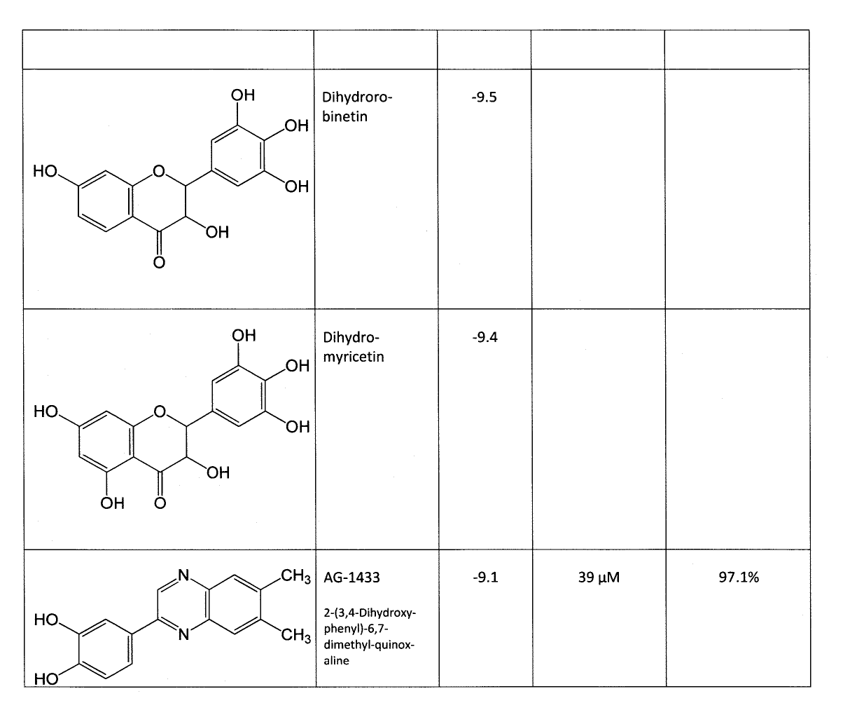

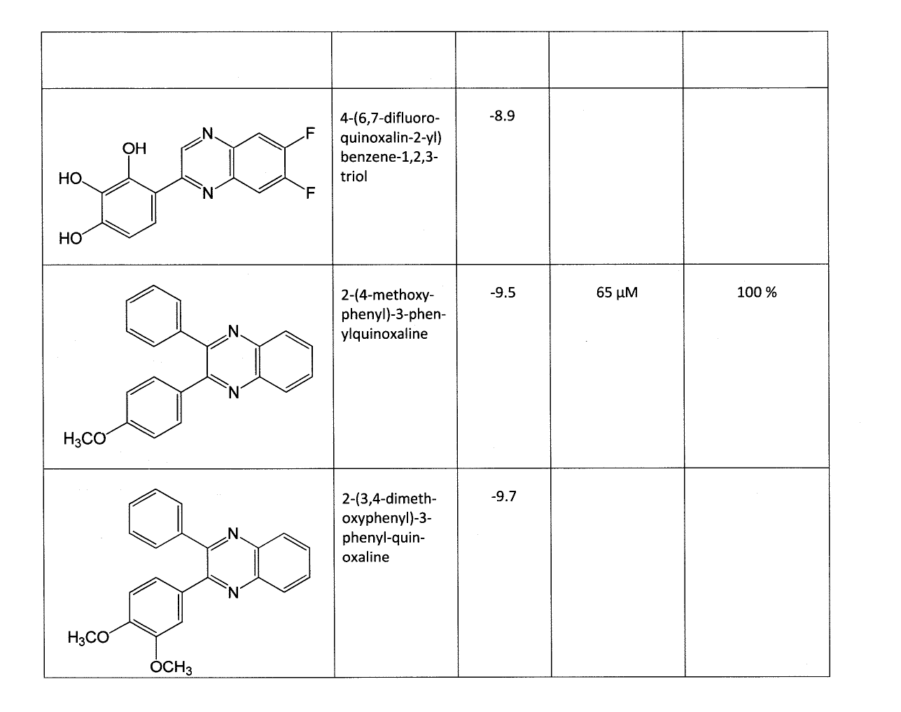

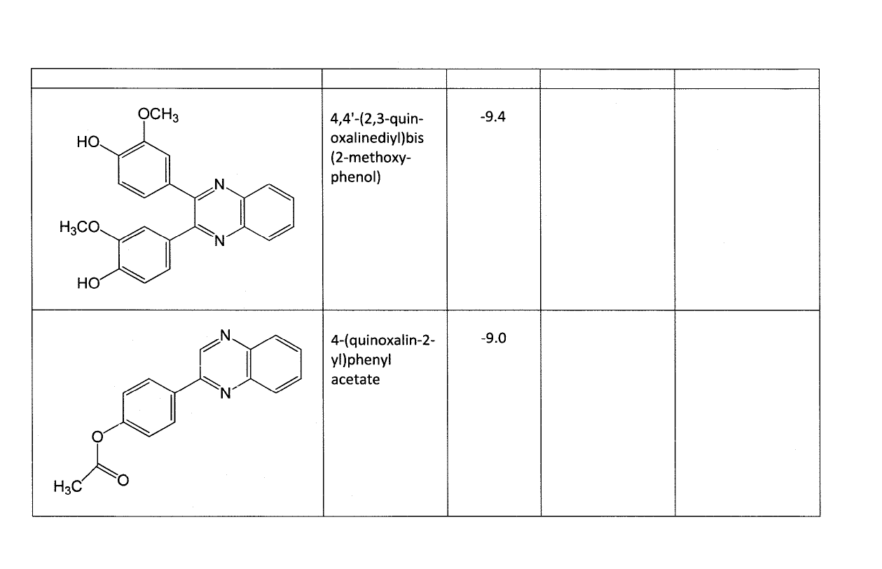

FIG. 3: Selective Inhibition of a C4-PEPC by folic acid (measured at a concentration of 0.025 mM)

The following Examples shall merely illustrate the invention. They shall not be construed, whatsoever, to limit the scope of the invention.

### EXAMPLE 1

The histidine-tagged PEP carboxylase from Flaveria pringlei or from Flaveria trinervia was heterologously expressed in E. coli and purified via immobilized metal affinity chromatography (IMAC). Crystals of the C3 (F. pringlei) and the C4 (F. trinervia) PEP carboxylase were obtained from the purified proteins by micro batch vapor diffusion. Crystals where analyzed by synchrotron radiation and PEPC structures were determined from the diffraction data by molecular replacement using XDS, coot and the CCP4 suite (Kabsch, 2010; Emsley et al. 2004; CCP4, 1994).

Crystal structures of PEP carboxylase from Flaveria pringlei or from Flaveria trinervia were superimposed by the align algorithm of PyMOL (DeLano, 2002) which performs a BLAST-like BLOSUM62-weighted dynamic programming sequence alignment followed by a series of refinement cycles intended to improve the fit by eliminating pairing with high relative variability. The malate binding site in the aligned crystal structures was visually inspected for structural differences in the region of 10-15 Å around the bound aspartate inhibitor. From this alignment the structural difference in position 884 was identified. The C3 type PEP carboxylase of Flaveria pringlei has a voluminous arginine side chain in this position, while the C4 type PEP carboxylase of Flaveria trinervia carries a small glycine residue in the corresponding position.

Potential selective inhibitors of C4 PEP carboxylase were selected from a Virtual Drug Screening (VDS) approach using the program PyRX (Wolf, 2009), standard compound libraries (ChemBank, NCI DataBase, KEGG Database), a library assembled from the Plant Metabolome Database and the high resolution crystal structures of PEP carboxylases from F. pringlei and F. trinervia.

In particular, the following potential selective inhibitors of C4 PEP carboxylase were identified:
1. (+)-Catechin [(2R,3S)-2-(3,4-dihydroxyphenyl)-3,4-dihydro-2H-chromene-3,5,7-triol]
2. Epigallocatechin gallate
3. 2-(4-methoxy-phenyl)-3-phenylquinoxaline
4. Folic Acid
5. 1-(3′-carboxy-4′-hydroxyphenyl)-2-(2,5-dihydroxyphenyl)ethane
6. 1(R),9(S)-hydrastine
7. 1(S),9(R)-beta-hydrastine
8. 2-(3,4-dihydroxyphenyl)-3,5,7-chromanetriol
9. 2-(3,4-Dihydroxyphenyl)-6,7-dimethylquinoxaline
10. 4-((12,20-dioxopregnan-3-yl)oxy)-4-oxobutanoic acid
11. Acacetin [5,7-dihydroxy-2-(4-methoxyphenyl)chromen-4-one]
12. Afrormosin
13. Apigenin [5,7-Dihydroxy-2-(4-hydroxyphenyl)-4H-1-benzopyran-4-one]
14. Chrysin [5,7-dihydroxy-2-phenyl-4H-chromen-4-one]
15. Daidzein [7-Hydroxy-3-(4-hydroxyphenyl) chromen-4-one]
16. Genistein [5,7-Dihydroxy-3-(4-hydroxyphenyl)chromen-4-one]
17. Hydrochlorothiazide
18. Malvidin[3,5,7-trihydroxy-2-(4-hydroxy-3,5-dimethoxyphenyl)chromenium]
19. N-((1.3-dioxo-1.3-dihydro-2H-isoindol-2-yl)acetyl)aspartic acid
20. N-((4-(((2.4-diamino-6-pteridinyl)methyl)amino)phenyl)acetyl)aspartic acid
21. N-((4-(((2-amino-4-hydroxy-6-pteridinyl)methyl)amino)phenyl)acetyl)glutamic acid
22. N-((5-(((2-amino-4-hydroxy-6-pteridinyl)methyl)amino)-2-thienyl)carbonyl)glutamic acid
23. N-([1.1′-biphenyl]-4-ylcarbonyl)glutamic acid
24. N-(2-quinoxalinylcarbonyl)aspartic acid
25. N-(2-quinoxalinylcarbonyl)glutamic acid
26. N-(4-((((2.4-diamino-6-pteridinyl)methyl)(methyl)amino)methyl)benzoyl)glutamic acid
27. N-(4-(((2,4-diamino-6-pteridinyl)methyl)amino)-3-methylbenzoyl)glutamic acid
28. N-(4-(((2,4-diamino-6-pteridinyl)methyl)amino)benzoyl)glutamic acid
29. N-(4-(((2,4-diamino-6-quinazolinyl)methyl)(methyl)amino)benzoyl)glutamic acid
30. N-(4-(((2,6-diamino-9H-purin-8-yl)methyl)(methyl)amino)benzoyl)glutamic acid
31. N-(4-(((2.4-diamino-6-pteridinyl)methyl)(methyl)amino)-3-iodobenzoyl)glutamic acid
32. N-(4-(((2-amino-4-hydrox-6-pteridinyl)methyl)amino)-3-iodobenzoyl)glutamic acid
33. N-(4-(((2-(2.4-diamino-6-pteridinyl)ethyl)(methyl)amino)benzoyl)glutamic acid
34. N-(4-(((2-(2-amino-4-hydroxy-6-pteridinyl)ethyl)(methyl)amino)benzoyl)glutamic acid
35. Naringenin [5,7-dihydroxy-2-(4-hydroxyphenyl)chroman-4-one]
36. Tectorigenin [5,7-dihydroxy-3-(4-hydroxyphenyl)-6-methoxychromen-4-one]
37. α-Cyano-(3,4-dihydroxy)cinnamoyl-(3′,4′-dihydroxyphenyl) ketone

### EXAMPLE 2

### Confirmation of Selective Inhibition by (+)-Catechin

Inhibition of purified PEP carboxylases from F. pringlei and F. trinervia by compounds selected from the VDS was monitored by a spectrophotometric coupled assay with malate dehydrogenase which reduces the oxaloacetate formed by PEP carboxylase to malate. The simultaneous oxidation of NADH is followed at 340 nm with standard optical equipment. The concentration of the inhibitors selected from the VDS was varied in the coupled assay to determine the half maximal inhibitory concentration (IC50).

With (+)-catechin the inhibition shown in FIG. 2 of the PEP carboxylase activity of the C3 and the C4 enzyme was obtained. Further results are given in table 4.

### EXAMPLE 3

### Confirmation of Selective Inhibition by Folic Acid

Inhibition of purified PEP carboxylases from F. pringlei and F. trinervia by compounds selected from the VDS was monitored by a spectrophotometric coupled assay with malate dehydrogenase which reduces the oxaloacetate formed by PEP carboxylase to malate. The simultaneous oxidation of NADH is followed at 340 nm with standard optical equipment. The concentration of the inhibitors selected from the VDS was varied in the coupled assay to determine the half maximal inhibitory concentration (1050).

With folic acid the inhibition shown in FIG. 3 of the PEP carboxylase activity of the C3 and the C4 enzyme was obtained.

### REFERENCES

Collaborative Computational Project, Number 4 (1994) The CCP4 Suite: Programs for Protein Crystallography. Acta Cryst. D50, 760-763.
DeLano, W. L., (2002) The PyMOL Molecular Graphics System, DeLano Scientific, San Carlos, Calif., USA
Doyle, J R, Burnell, J N, Haines, D S, Llewellyn, L E, Motti, C A und Tapiolas, D M (2005) A Rapid Screening Method to Detect Specific Inhibitors of Pyruvate Orthophosphate Dikinase as Leads for C4 Plant-Selective Herbicides. J Biomol Screen 10: 67-75.
Durrant, J D, Amaro, R E und McCammon, J A (2009) AutoGrow: A Novel Algorithm for Protein Inhibitor Design. Chemical Biology & Drug Design 73(2):168-178
Emsley P, Cowtan K (2004)Coot: model-building tools for molecular graphics. Acta Crystallographica Section D-Biological Crystallography 60: 2126-2132 Part 12 Sp. Iss.
Jenkins C L D, Harris R L N und McFadden H G (1987) 3,3-Dichloro-2-dihydroxyphosphinoylmethyl-2-propenoate, a new, specific inhibitor of phosphoenolpyruvate carboxylase. Biochem. Int 14:219-226.
Jenkins C L D (1989) Effects of the phosphoenolpyruvate carboxylase inhibitor 3,3-dichloro-2-(dihydroxyphosphinoylmethyl)propenoate on photosynthesis. Plant Physiol 89:1231-1237.
Kabsch, W. (2010) XDS. Acta Cryst. D66, 125-132
Mancera R L, Gómez A G und Pisanty A. (1995) Quantitative structure-activity relationships of com-petitive inhibitors of phosphoenolpyruvate carboxylase. Bioorg Med Chem. 3(3):217-25.
Matsumura, H, Xi; Y, Shirakata, S, Inoue, T, Yoshinaga, T, Ueno, Y, Izui, K und Kai Y. (2002) Crystal structures of C4 form maize and quaternary complex of E. coli phosphoenolpyruvate car-boxylases. Structure 10(12):1721-30.
McFadden, H G, Harris, R L N and Jenkins, C L D (1989) Potential Inhibitors of Phosphoenolpyruvate Carboxylase. II. Phosphonic Acid Substrate Analogues Derived from Reaction of Trialkyl Phosphites with Halomethacrylates. Aust. J. Chem. 42:301-14.
Motti, C A, Bourne, D G, Burnell, J N, Doyle, J R, Haines, D S, Liptrot, C H, Llewellyn, L E, Ludke, S, Muirhead, A und Tapiolas D M (2007) Screening marine fungi for inhibitors of the C4 plant enzyme pyruvate phosphate dikinase: unguinol as a potential novel herbicide candidate. Appl Environ Mi-crobiol. 73(6):1921-7
Pairoba, C F, Colombo, S L and Andreo, C S (1996) Flavonoids as Inhibitors of NADP-Malic Enzyme and PEP Carboxylase from C4 Plants. Biosci. Biotech, Biochem. 60(5): 779-783.
Trott, O und Olson, A J (2010) AutoDock Vina: improving the speed and accuracy of docking with a new scoring function, efficient optimization and multithreading. Journal of Computational Chemistry 31:455-461.
Wolf L K (2009) New software and Websites for the Chemical Enterprise, Chemical & Engineering News 87, 31

1. A method of inhibiting the growth of a C4 plant, the method comprising applying a compound, a salt or solvate thereof as a C4 plant selective herbicide, wherein said compound comprises a cyclic alkyl, aryl, heterocycloalkyl or heteroaryl group, and wherein said compound further comprises:
(a) at least one functional group Z being —C(═Y^{Z})R¹, wherein Y^{Z} is selected from the group consisting of O, NH and S, preferably wherein Y^{Z} is O, and
wherein R¹ is selected from the group consisting of H, OH and N(R²R³), —O(R²) and —S(R²), wherein R² and R³, are independently of each other, selected from the group consisting of H, Alkyl, cycloalkyl, aryl and heteroaryl and a functional group Q which is —C(═Y^{Q})R⁴, wherein Y^{Q} is selected from the group consisting of O, NH and S, preferably wherein Y^{Q} is O, and wherein R⁴ is selected from the group consisting of H, OH and NR^{4#}R^{5#}, OR^{4#}, and SR^{4#}, wherein R^{4#} and R^{5#}, are independently of each other, selected from the group consisting of H, alkyl, cycloalkyl, aryl and heteroaryl, or
(b) at least one electron donating group selected from the group consisting of OH, —SH, —O—R^{p}, —S—R^{p}, —NR^{p}R^{q} and —O—C(═Y^{p})R^{r}, wherein R^{p} and R^{q} are, independently of each other, selected from the group consisting of H, alkyl, cycloalkyl and heteroaryl, and wherein R^{r} is —OH, —NH₂, —NH-Alkyl, —O-Alkyl or —NH-Alkyl, and wherein Y^{p} is selected from the group consisting of —S—, —O—, —NH,
wherein said compound binds to the malate binding site comprised by a phosphoenolpyruvate carboxylase from a C4 plant and thereby inhibits said phosphoenolpyruvate carboxylase, and
wherein the cyclic alkyl, aryl, heterocycloalkyl or heteroaryl group inhibits binding to the malate binding site of a phosphoenolpyruvate carboxylase from a C3 plant.

2. The method according to claim 1, wherein the compound has a structure according to the formula (I): 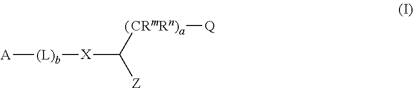
wherein A is the cyclic alkyl, aryl, heterocycloalkyl, or heteroaryl group and wherein A is at least a bicyclic alkyl, aryl, or heteroaryl group, preferably a bicyclic aryl or heteroalkyl group,
X is a functional group, preferably X is —Y^{X}—, —C(═Y^{XX})—, wherein Y^{X} is selected from the group consisting of —S—, —O—, —NH—, —N(CH₃)— and NH—NH—, and Y^{XX} is selected from the group consisting of —S—, —O—, —NH,
and wherein in case X is —Y^{X}—, F¹ is —C(═Y^{F})— or —C(═Y^{F})—NR^{Y1} and wherein in case X is —C(═Y^{X})—, —C(═Y^{X})—NR^{X}—, F¹ is Y^{F1}—,
in particular —NH— or —C(═Y)— with Y being S or O,
L is a linking moiety, preferably a linking moiety comprising an aryl group, integer b is 0 or 1,
integer a is in the range of from 2 to 4,
and wherein R^{m} and Rⁿ are, independently of each other, H or alkyl, preferably H or methyl, in particular H.

3. The Method according to claim 2, wherein Q is COOH, the compound having the structure (II):

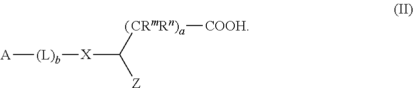

4. The method according to claim 1, wherein Z is COOH, the compound having the structure (III):

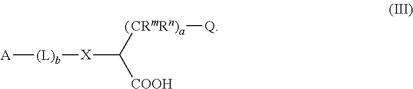

5. The method according to any of claim 2, wherein L has a structure according to the following formula
-[F²]_{c}-[L¹]_{d}-[F¹]ₑ-
wherein L¹ is a linking moiety, preferably selected from the group consisting of alkyl, alkenyl, alkylaryl, arylalkyl, aryl, heteroaryl, alkylheteroaryl and heteroarylalkyl, and wherein integer d is 0 or 1, and
wherein F¹ is a functional group selected from the group consisting of —Y^{F1}—, —C(═Y^{F})—, and —C(═Y^{F})—NR^{Y1}—,
wherein Y^{F1} is selected from the group consisting of —S—, —O—, —NH—, —N(CH₃)— and NH—NH—,
Y^{F} is selected from the group consisting of —S—, —O—, and —NH,
R^{Y1} is H or alkyl,
and wherein in case X is —Y^{X}—, F¹ is —C(═Y^{F})— or —C(═Y^{F})—NR^{Y1} and wherein in case X is —C(═Y^{X})—, —C(═Y^{XX})—NR^{X}—, F¹ is —Y^{F1}—,
integer e is 0 or 1,
F² is a functional group selected from the group consisting of —Y^{F2}—, —C(═Y^{FF})—, —C(═Y^{FF})—NR^{Y2}— and —NR^{Y2}—C(═Y^{FF})—,
wherein Y^{F2} is selected from the group consisting of —S—, —O—, —NH—, —N(CH₃)—, and —NH—NH—,
YF^{F} is selected from the group consisting of —S—, —O—, and —NH,
R^{Y2} is H or alkyl, and
wherein integer c is 0 or 1.

6. The method according to claim 5, wherein X is —NH— and wherein L is
—Y^{F2}-L¹-C(═Y^{F})—
wherein L¹ is alkylaryl, arylalkyl, heteroaryl or aryl group, preferably a group having one of the following structures: 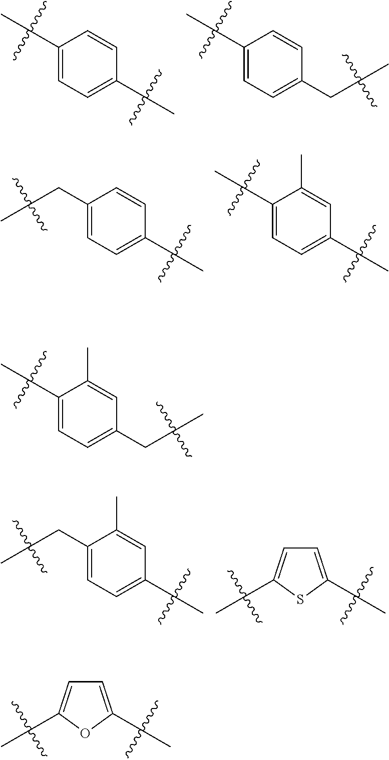
more preferably wherein L is selected from the following structures:

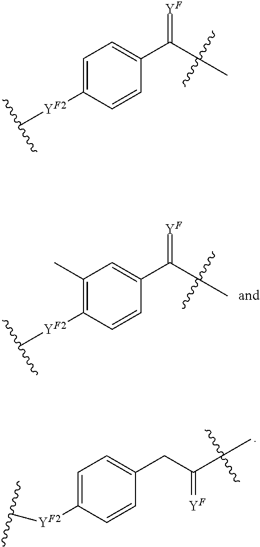

7. The method according to claim 2, wherein A is selected from the group consisting of:

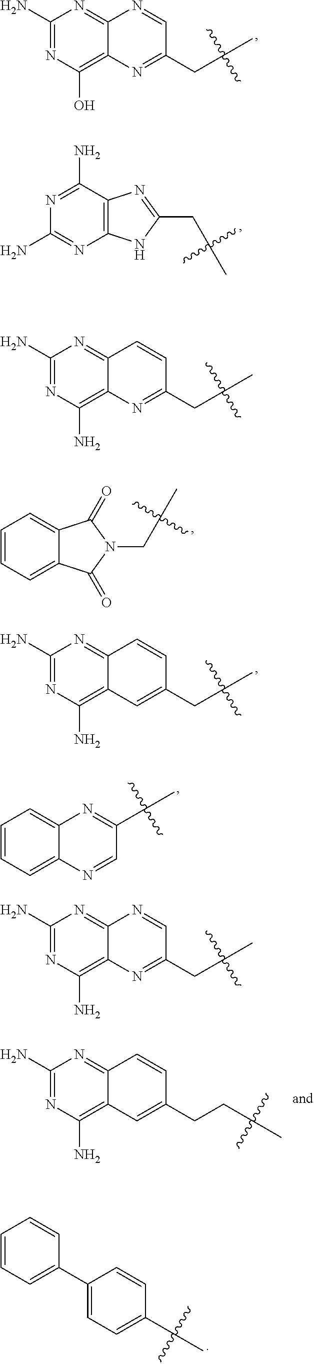

8. The method according to claim 1, wherein the compound has a structure of the formula (IV′) or (IV″): 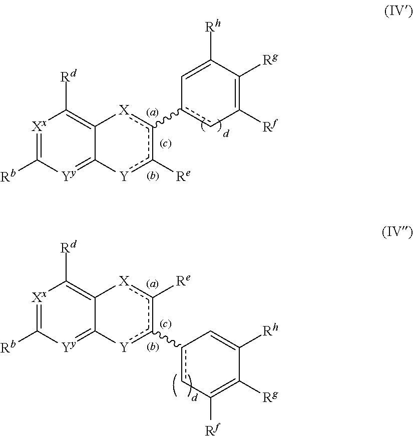
wherein
X is selected from the group consisting of —C(═O)—, —O— and —N—,
Y is selected from the group consisting of —C(H)ₚ—, —C(═O)—, —O— and —N—,
X^{x} is selected from the group consisting of —C(R^{a})— or N, Y^{y} is selected from the group consisting of —C(R^{c})— or N,
with p being 1 or 2,
the bond (a) is a single or double bond,
the bond (b) is a single or double bond,
the bond (c) is a single or double bond,
and wherein R^{a} and R^{b} are, independently of each other selected from the group consisting of H, Alkyl, halogene, —C(═Y^{p})R^{r}, and an electron donating group, wherein R^{r} is —OH, —NH₂, —NH-Alkyl, —O-Alkyl, or —S-Alkyl, and wherein Y^{p} is selected from the group consisting of —S—, —O—, and —NH, or wherein R^{a} and R^{b} form together a cycloalkyl, cycloheteroalkyl, aryl or heteroaryl, wherein the cycloalkyl, cycloheteroalkyl, aryl or heteroaryl is a single-ring group or a multicyclic group,
and wherein R^{c}, R^{d}, R^{f}, R^{g} and R^{h} are, independently of each other, selected from the group consisting of H, Alkyl, halogene, —C(═Y^{p})R^{r}, and an electron donating group, wherein R^{r} is —OH, —NH₂, —NH-Alkyl, —O-Alkyl, or —S-Alkyl, and wherein Y^{p} is selected from the group consisting of —S—, —O—, and —NH,
and wherein R^{e} is selected from the group consisting of H, Alkyl, halogene, —C(═Y^{p})R^{r}, —O—C(═Y^{p})R^{r}, an electron donating group, aryl, heteroaryl group and —O—C(═O)—R*, wherein R* is an aryl or heteroaryl group, and wherein R^{r} is —OH, —NH₂, —NH-Alkyl, —O-Alkyl, or —S-Alkyl, and wherein Y^{p} is selected from the group consisting of —S—, —O—, and —NH,
wherein at least one of R^{a}, R^{b}, R^{c}, R^{d}, R^{f}, R^{g}, R^{e} and R^{h} is an electron donating group, with the proviso that in case the bond (c) is a double bond, (a) and (b) are both single bonds, and in case X is —C(═O)—, the bond (a) is a single bond, and wherein in case Y is —C(═O)—, the bond (b) is a single bond.

9. The method according to claim 8, wherein the compound has a structure of the formula (IV′) or (IV″): 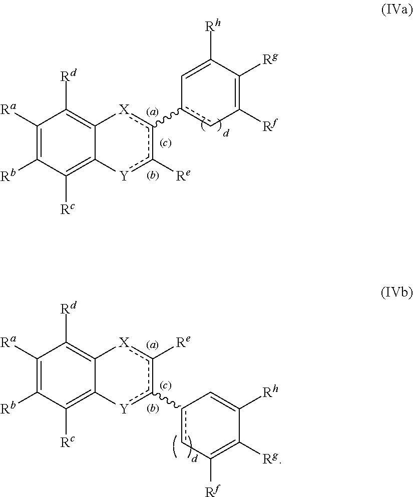
preferably wherein R^{a}, R^{h}, R^{c}, R^{d}, R^{e}, R^{f}, R^{g} and R^{h}, independently of each other, are selected from the group consisting of H, Alkyl, —C(═Y^{p})R^{r}, and an electron donating group, said electron donating group being selected from the group consisting of —OH, —SH, —O-Alkyl, —S-Alkyl, —NR^{p}R^{q} and —C(═Y^{p})R^{r}, wherein R^{p} and R^{q} are, independently of each other H or alkyl,
and wherein R^{r} is —OH, —NH₂, —NH-Alkyl, —O-Alkyl or —NH-Alkyl,
and wherein Y^{p} is selected from the group consisting of —S—, —O—, and —NH,
wherein at least one of R^{a}, R^{b}, R^{c}, R^{d}, R^{e} and R^{f} is an electron donating group.

10. The method according to claim 8, wherein at least one of R^{a}, R^{b}, R^{c}, R^{d}, R^{e} and R^{f} is —OH.

11. A method for identifying a C4 plant selective herbicide, comprising screening a compound library for a compound which is:
i) capable of binding to a malate binding site comprised by a phosphoenolpyruvate carboxylase from a C4 plant, thereby inhibiting the activity of said phosphoenolpyruvate carboxylase, and
ii) not capable of binding to a malate binding site comprised by a phosphoenolpyruvate carboxylase from a C3 plant.

12. The method of claim 11, wherein screening the compound library comprises the steps of:
(a) providing the three-dimensional structure of the malate binding site of a phosphoenolpyruvate carboxylase from a C4 plant,
(b) providing the three-dimensional structure of the malate binding site phosphoenolpyruvate carboxylase from a C3 plant,
(c) providing a three-dimensional structure of the compound to be tested, and
(d) identifying, based on the three-dimensional structures provided in steps (a), (b), and/or (c), a compound which is capable of binding to the malate binding site comprised by a phosphoenolpyruvate carboxylase from a C4 plant, but which is not capable of binding to the malate binding site comprised by a phosphoenolpyruvate carboxylase from a C3 plant.

13. The method of claim 11, further comprising the steps of:
i) contacting a compound comprised by the compound library to be tested with a phosphoenolpyruvate carboxylase from a C3 plant, and assessing whether said compound binds to said malate binding site of said phosphoenolpyruvate carboxylase from said C3 plant, and
ii) contacting said compound with a phosphoenolpyruvate carboxylase from a C4 plant, and assessing whether said compound binds to said malate binding site of said phosphoenolpyruvate carboxylase from said C4 plant.

14. The method of claim 13, wherein assessing whether said compound binds to said malate binding site of phosphoenolpyruvate carboxylase from said C3 or C4 plant is done by determining enzyme activity of said phosphoenolpyruvate carboxylase after contacting the compound with the phosphoenolpyruvate carboxylase.

15. The method of claim 11, wherein the compound does not bind the malate binding site comprised by a phosphoenolpyruvate carboxylase from a C3 plant due to steric and/or electrostatic interactions with the conserved arginine residue comprised by said malate binding site.

16. The method of claim 11, wherein the phosphoenolpyruvate carboxylase from the C4 plant is derived from Flaveria trinervia, and/or wherein the phosphoenolpyruvate carboxylase from the C3 plant is derived from Flaveria pringlei.
